(19) United States
(12) Patent Application Publication
(10) Pub. No.: US 2023/0357419 A1
(43) Pub. Date: Nov. 9, 2023

(54) METHODS FOR TREATING OR PREVENTING HEADACHE DISORDERS

(71) Applicants: Yu-Qing Cao, St. Louis, MO (US); Xuemei Liu, St. Louis, MO (US); Sun Ryu, St. Louis, MO (US)

(72) Inventors: Yu-Qing Cao, St. Louis, MO (US); Xuemei Liu, St. Louis, MO (US); Sun Ryu, St. Louis, MO (US)

(73) Assignee: Washington University, St. Louis, MO (US)

(21) Appl. No.: 18/311,991

(22) Filed: May 4, 2023

Related U.S. Application Data
(60) Provisional application No. 63/338,105, filed on May 4, 2022.

(51) Int. Cl. C07K 16/28; A61P 25/06; C07K 16/24; A61K 31/537; C07K 16/18

(52) U.S. Cl. CPC C07K 16/2866; A61P 25/06; C07K 16/24; A61K 31/537; C07K 16/18

(57) ABSTRACT

The present disclosure provides for methods of treating or preventing a headache disorder in a subject including administering to the subject a CCL2-CCR2 signaling inhibiting agent.

### CROSS-REFERENCE TO RELATED APPLICATIONS

This application claims priority from U.S. Provisional Application Ser. No. 63/338,105 filed on 4 May 2022, which is incorporated herein by reference in its entirety.

### STATEMENT REGARDING FEDERALLY SPONSORED RESEARCH OR DEVELOPMENT

This invention was made with government support under W81XWH-18-1-0627 awarded by the Army Medical Research and Materiel Command (ARMY/MRMC) and under NS103350 awarded by the National Institutes of Health. The government has certain rights in the invention.

### MATERIAL INCORPORATED-BY-REFERENCE

Not applicable.

### FIELD

The present disclosure generally relates to methods for treating or preventing headache disorders using CCL2-CCR2 signaling inhibiting agents.

### SUMMARY

In an aspect of the present disclosure, a method of treating or preventing a headache disorder in a subject in need thereof is provided. The method comprises administering to the subject a chemokine CC motif ligand 2 (CCL2)-CC chemokine receptor type 2 (CCR2) signaling inhibiting agent.

In some embodiments, the headache disorder is migraine, chronic migraine, chronic headache, stress-induced headache, acute post-traumatic headache (PTH), or chronic PTH. In some embodiments, the subject has suffered a mild traumatic brain injury (mTBI). In some embodiments, the CCL2-CCR2 signaling inhibiting agent is an anti-CCL2 or anti-CCR2 antibody. In some embodiments, the CCL2-CCR2 signaling inhibiting agent is a bispecific antibody against calcitonin gene-related peptide (CGRP) and one of CCL2 or CCR2. In some embodiments, the CCL2-CCR2 signaling inhibiting agent is a small molecule antagonist of CCR2, for example, the small molecule antagonist is RS504393. In some embodiments, the method further comprises administering to the subject a CGRP inhibiting agent, the CGRP inhibiting agent or the CCL2-CCR2 signaling inhibiting agent is administered to the subject at a sub-effective dose, and administering the CGRP inhibiting agent or the CCL2-CCR2 signaling inhibiting agent at a sub-effective dose reduces, prevents, or reverses mechanical hypersensitivity or hyperalgesic priming in the subject. In some embodiments, the CGRP inhibiting agent is an anti-CGRP antibody. In some embodiments, the administering the CCL2-CCR2 signaling inhibiting agent to the subject reduces, prevents, or reverses mechanical hypersensitivity or hyperalgesic priming in the subject.

In another aspect of the present disclosure, a method of inhibiting chemokine CC motif ligand 2 (CCL2)-CC chemokine receptor type 2 (CCR2) signaling in a subject having a headache disorder is provided. The method comprises administering to the subject a CCL2-CCR2 signaling inhibiting agent.

In some embodiments, the headache disorder is migraine, chronic migraine, chronic headache, stress-induced headache, acute post-traumatic headache (PTH), or chronic PTH. In some embodiments, the CCL2-CCR2 signaling inhibiting agent is an anti-CCL2 or anti-CCR2 antibody. In some embodiments, the CCL2-CCR2 signaling inhibiting agent is a bispecific antibody against calcitonin gene-related peptide (CGRP) and CCL2 or CCR2. In some embodiments, the CCL2-CCR2 signaling inhibiting agent is a small molecule antagonist of CCR2, for example, the small molecule antagonist is RS504393. In some embodiments, the method further comprises administering to the subject a CGRP inhibiting agent. In some embodiments, administering the CCL2-CCR2 signaling inhibiting agent to the subject reduces, prevents, or reverses mechanical hypersensitivity or hyperalgesic priming in the subject.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1F is an exemplary embodiment showing the effects of repeated NTG administration on mast cells, neutrophils and B cells in mouse dura and TG in accordance with the present disclosure. FIG. 1A is a bar graph showing the density of Ly6G⁺ neutrophils in the dura (surrounding the MMA) from female C57BL/6J mice after 5 repetitive i.p. injections of vehicle (Veh) or NTG (10 mg/kg, n=4-5 mice/group). FIG. 1B is a bar graph showing the density of CD19⁺ B cells in the dura of Veh- and NTG-treated mice (same as in FIG. 1A). FIG. 1C and FIG. 1D are bar graphs showing the density of total mast cells (FIG. 1C) and the percentage of degranulated mast cells (FIG. 1D) in dura surrounding the MMA from female C57BL/6J mice after 5 repetitive Veh or NTG injections (n=4 mice/group). **P<0.01, two-tailed test. FIG. 1E and FIG. 1F are bar graphs showing the number of total mast cells (FIG. 1E) and the percentage of degranulated mast cells (FIG. 1F) in the TG of Veh- or NTG-treated mice (same as in FIG. 1C and FIG. 1D).

FIG. 2A-FIG. 2F is an exemplary embodiment showing repeated NTG administration increases the density of macrophages as well as the expression of Ccl2 and Ccr2 mRNA in dura and TG in accordance with the present disclosure. FIG. 2A includes representative images of Iba1⁺ macrophages in the dura and TG of female C57BL/6J mice after 5 repetitive i.p. injections of vehicle (Veh) or NTG. FIG. 2B is a bar graph showing the densities of Iba1⁺ macrophages in the dura (surrounding the MMA but not adjacent to the blood vessels), TG and L4 DRG from Veh- and NTG-treated female C57BL/6J mice (n=3-6 mice/group). ***P<0.001, two-tailed test. FIG. 2C is a bar graph showing the cross-sectional area of individual Iba1⁺ macrophages in the dura (surrounding the MMA but not adjacent to the blood vessels), TG and L4 DRG from Veh- and NTG-treated mice, respectively (same mice as in FIG. 2B). *P<0.05, two-tailed test. FIG. 2D is a bar graph showing the density of Iba1-ir in the dura immediately adjacent to MMA (same mice as in FIG. 2B). *P<0.05, two-tailed test. FIG. 2E and FIG. 2F are bar graphs showing relative Ccl2 (FIG. 2E) and Ccr2 (FIG. 2F) mRNA expression levels in the dura and TG from female CD-1 mice after 5 repetitive i.p. injections of Veh or NTG (n=5 mice/group). Tissues were collected 2 days after the last injection. The abundance of Ccl2 and Ccr2 mRNA was normalized to that of GAPDH in individual samples. *P<0.05, two-tailed t-test.

FIG. 3A-FIG. 3D is an exemplary embodiment showing Ccl2 and Ccr2 global KO mice do not develop NTG-induced behavioral sensitization or hyperalgesic priming in accordance with the present disclosure. FIG. 3A is a timeline of experiments. Note that NTG was injected after the completion of behavioral tests on the same day. In some mice, the facial mechanical thresholds were measured 2 hours after the 1st NTG injection as indicated by the grey arrow. FIG. 3B is a bar graph showing the 50% withdrawal thresholds to von Frey filaments at the periorbital region of male wild-type (WT), Ccl2 KO, and Ccr2 KO mice before and 2 hours after the first NTG injection (n=4-5 mice/group). Two-way ANOVA: P<0.001 for genotype (F_{[2, 13]}=17.68), treatment (F_{[1, 13]}=37.26), and genotype×treatment interaction (F_{[2, 13]}=22.00); post hoc Bonferroni test: ***P<0.001. FIG. 3C is a line graph showing the effects of repeated NTG on facial mechanical thresholds of male wild-type and Ccl2 KO mice (n=5-6 mice/group). Two-way RM ANOVA: P<0.001 for group (F_{[1, 55]}=89.68), time (F_{[5, 55]}=11.96) and group×time interaction (F_{[5, 55]}=9.36); post hoc Student-Newman-Keuls test: ***P<0.001, between the corresponding wild-type and Ccl2 KO groups; ^{###}P<0.001, compared with the baseline (day 1) threshold in the wild-type group. FIG. 3D is a line graph showing the effects of repeated NTG on facial mechanical responses of male wild-type, Ccr2 KO, and Ccr2 heterozygous (HZ) mice (n=4-8 mice/group). Two-way RM ANOVA: P<0.001 for group (F_{[2, 80]}=70.57), time (F_{[5, 80]}=47.99) and group×time interaction (F_{[10, 80]}=8.97); post hoc Student-Newman-Keuls test: ***P<0.001, wild-type versus Ccr2 KO; ^{†††}P<0.001, Ccr2 HZ versus Ccr2 KO; ^{###}P<0.001, compared with the baseline threshold in the wild-type group; ^{∧∧∧}P<0.001, compared with the baseline threshold in the Ccr2 HZ group.

FIG. 4A-FIG. 4F is an exemplary embodiment showing the effects of genetic or pharmacological inhibition of CCL2-CCR2 signaling pathway on repeated NTG-induced behavioral sensitization in accordance with the present disclosure. FIG. 4A is a line graph showing the effects of repeated vehicle (Veh) or NTG administration on facial mechanical thresholds in female wild-type and Ccl2 KO mice (n=7-10 mice/group). Two-way RM ANOVA: P<0.001 for group (F_{[3, 165]}=89.17), time (F_{[5, 165]}=6.11) and group×time interaction (F_{[15, 165]}=6.65); post hoc Student-Newman-Keuls test: ***P<0.001, wildtype+NTG versus Ccl2 KO+NTG; ^{§§§} P<0.001, wild-type+Veh versus wild-type+NTG; ^{###}P<0.001, compared with the baseline (day 1) threshold in the wild-type+NTG group. FIG. 4B is a line graph showing the effects of repeated NTG on facial mechanical responses of female wild-type and Ccr2 KO mice (n=5-7 mice/group). Two-way RM ANOVA: P<0.001 for group (F_{[1, 60]}=387.2), time (F_{[5, 60]}=33.2) and group×time interaction (F_{[5, 60]}=24.4); post hoc Student-Newman-Keuls test: ***P<0.001, between the corresponding wild-type and Ccr2 KO groups; ^{###}P<0.001, compared with the baseline (day 1) threshold in the wild-type group. FIG. 4C is a timeline of the experiments in FIG. 4D. FIG. 4D is a bar graph showing the effects of periorbital injections of vehicle or RS504393 on facial mechanical responses at baseline (BL) as well as 3 hours (3h) and 2 days (48h) post-NTG administration (n=5 female C57BL/6J mice/group). One-way RM ANOVA: P<0.001 for vehicle (F_{[2, 10]}=68.04), and RS504393 groups (F_{[2, 10]}=124.0); post-hoc Student-Newman-Keuls test: ***P<0.001, compared with the BL thresholds. FIG. 4E is a line graph showing the effects of delayed, 2 times of CCL2 ab treatment on NTG-induced behavioral sensitization and hyperalgesic priming in male C57BL/6J mice (n=7 mice/group). Day 1-20, two-way RM ANOVA: P<0.001 for group (F_{[1, 112]}=44.38), time (F_{[8, 112]}=32.14), and group×time interaction (F_{[8, 112]}=13.44); post-hoc Student-Newman-Keuls test: **P<0.01, ***P<0.001, control IgG versus CCL2 ab; ^{#}P<0.05, ^{###}P<0.001, compared with the baseline (day 1) threshold in the control IgG group; ^{†††}p<0.001, compared with the baseline threshold in the CCL2 ab group. Day 20-22, two-way RM ANOVA: P<0.001 for group (F_{[1, 14]}=3.65), time (F_{[1, 14]}=67.59), and group×time interaction (F_{[1, 14]}=11.78); post-hoc Student-Newman-Keuls test: **P<0.01, control IgG versus CCL2 ab; ^{###}P<0.001, compared with the day 20 threshold in the control IgG group; ^{††}P<0.01, compared with the day 20 threshold in the CCL2 ab group. FIG. 4F is a line graph showing the effects of delayed, 3 times of CCL2 ab treatment on NTG-induced behavioral sensitization and hyperalgesic priming in male C57BL/6J mice (n=7 mice/group, same control IgG group as in FIG. 4E). Day 1-20, two-way RM ANOVA: P<0.001 for group (F_{[1, 112]}=113.7), time (F_{[8, 112]}=38.8), and group×time interaction (F_{[8, 112]}=14.5); post-hoc Student-Newman-Keuls test: ***P<0.001, control IgG versus CCL2 ab; ^{#}P<0.05, ^{###}P<0.001, compared with the baseline (day 1) threshold in the control IgG group; ^{†††}p<0.001, compared with the baseline threshold in the CCL2 ab group. Day 20-22, two-way RM ANOVA: P<0.001 for group (F_{[1, 14]}=16.42), time (F_{[1, 14]}=48.56), and group×time interaction (F_{[1, 14]}=7.95); posthoc Student-Newman-Keuls test: ***P<0.001, control IgG versus CCL2 ab; ^{###}P<0.001, compared with the day 20 threshold in the control IgG group; ^{†}P<0.05, compared with the day 20 threshold in the CCL2 ab group.

FIG. 5A-FIG. 5B is an exemplary embodiment showing Ccl2 and Ccr2 global KO mice do not develop NTG-induced behavioral sensitization or hyperalgesic priming in accordance with the present disclosure. FIG. 5A is a line graph showing changes of facial mechanical thresholds of female wild-type, Ccl2 KO, and Ccr2 KO mice in response to repeated high-dose NTG administration (n=5-8 mice/group). Two-way RM ANOVA: P<0.001 for group (F_{[2, 160]}=26.58), time (F_{[8, 160]}=9.27) and group×time interaction (F_{[16, 160]}=4.09); post hoc Student-Newman-Keuls test: ***P<0.001, wild-type versus Ccl2 KO; ^{†††}P<0.001, wild-type versus Ccr2 KO; ^{###}P<0.001, compared with the baseline (day 1) threshold in the wild-type group. FIG. 5B is a bar graph showing facial mechanical responses were measured before (day 20) and after (day 22) low-dose NTG injections to reveal hyperalgesic priming induced by prior repeated high-dose NTG (same mice as in FIG. 5A). Two-way ANOVA: P<0.001 for genotype (F_{[2, 20]}=1.46), treatment (F_{[1, 20]}=14.83), and genotype×treatment interaction (F_{[2, 20]}=3.94); post hoc Bonferroni test: ***P<0.001.

FIG. 6A-FIG. 6E is an exemplary embodiment showing peripheral CCL2-CCR2 signaling is essential for the development and/or expression of NTG-induced behavioral sensitization in accordance with the present disclosure. FIG. 6A is a timeline of the experiments in FIG. 6B-FIG. 6E. Note that the drugs were injected after the completion of behavioral tests on the same day. FIG. 6B is a line graph showing the effects of RS504393 on NTG-induced behavioral sensitization and hyperalgesic priming in female C57BL/6J mice (n=8 mice/group). Day 1-20, two-way RM ANOVA: P<0.001 for group (F_{[1, 128]}=56.64), time (F_{[8, 128]}=30.03), and group×time interaction (F_{[8, 128]}=13.78); post-hoc Student-Newman-Keuls test: ***P<0.001, between the corresponding vehicle and RS504393 groups; ^{#}P<0.05, ^{###}P<0.001, compared with the baseline (day 1) threshold in the vehicle group; ^{†††}P<0.001, compared with the baseline threshold in the RS504393 group. Day 20-28, two-way RM ANOVA: P<0.001 for group (F_{[1, 64]}=2.02), time (F_{[4, 64]}=60.59), and group×time interaction (F_{[4, 64]}=0.95); post-hoc Student-Newman-Keuls test: ^{###}P<0.001, compared with the day 20 threshold in the vehicle group; ^{†††}P<0.001, compared with the day 20 threshold in the RS504393 group. FIG. 6C is a line graph showing starting the CCL2 ab treatment before and during repeated high-dose NTG prevents the development of persistent sensitization and hyperalgesic priming in female C57BL/6J mice (n=7-8 mice/group). Day 1-20, two-way RM ANOVA: P<0.001 for group (F_{[1, 135]}=96.08), time (F_{[9, 135]}=20.83), and group×time interaction (F_{[9, 135]}=12.56); post-hoc Student-Newman-Keuls test: ***P<0.001, between the corresponding control IgG and CCL2 ab groups; ^{###}P<0.001, compared with the baseline (day 1) threshold in the control IgG group. Day 20-28, two-way RM ANOVA: P<0.001 for group (F_{[1, 48]}=60), time (F_{[4, 48]}=60), and group×time interaction (F_{[4, 48]}=60); post-hoc Student-Newman-Keuls test: ***P<0.001, between the corresponding control IgG and CCL2 ab groups; ^{###}P<0.001, compared with the day 20 threshold in the control IgG group. FIG. 6D is a line graph showing the effects of delayed CCL2 ab treatment on NTG-induced behavioral sensitization and hyperalgesic priming in female C57BL/6J mice (n=7-8 mice/group). Day 1-20, two-way RM ANOVA: P<0.001 for group (F_{[1, 120]}=46.76), time (F_{[8, 120]}=30.09), and group×time interaction (F_{[8, 120]}=13.71); post-hoc Student-Newman-Keuls test: **P<0.01, ***P<0.001, between the corresponding control IgG and CCL2 ab groups; ^{###}P<0.001, compared with the baseline (day 1) threshold in the control IgG group; ^{†††}p<0.001, compared with the baseline threshold in the CCL2 ab group. Day 20-28, two-way RM ANOVA: P<0.001 for group (F_{[1, 60]}=4.23), time (F_{[4, 60]}=33.27), and group×time interaction (F_{[4, 60]}=5.84); post-hoc Student-Newman-Keuls test: *P<0.05, between the corresponding control IgG and CCL2 ab groups; ^{###}P<0.001, compared with the day 20 threshold in the control IgG group; ^{††}P<0.01, compared with the day 20 threshold in the CCL2 ab group. FIG. 6E is a line graph showing the effects of prolonged CCL2 ab treatment on NTG-induced behavioral sensitization and hyperalgesic priming in male C57BL/6J mice (n=6 mice/group). Day 1-20, two-way RM ANOVA: P<0.001 for group (F_{[1, 96]}=25.29), time (F_{[8, 96]}=24.19), and group×time interaction (F_{[8, 96]}=13.07); post-hoc Student-Newman-Keuls test: *P<0.05, **P<0.01,***P<0.001, between the corresponding control IgG and CCL2 ab groups; ^{###}P<0.001, compared with the baseline (day 1) threshold in the control IgG group; ^{†††}P<0.001, compared with the baseline threshold in the CCL2 ab group. Day 20-28, two-way RM ANOVA: P<0.001 for group (F_{[1, 48]}=41.68), time (F_{[4, 48]}=9.55), and group×time interaction (F_{[4, 48]}=11.71); post-hoc Student-Newman-Keuls test: ***P<0.001, between the corresponding control IgG and CCL2 ab groups; ^{###}P<0.001, compared with the day 20 threshold in the control IgG group.

FIG. 7 is an exemplary embodiment showing identification of CCL2-expressing cells in mouse TG and dura in accordance with the present disclosure. FIG. 7 includes representative images of Ccl2⁺ neurons in a mouse TG section.

FIG. 8A-FIG. 8C is an exemplary embodiment showing RNAscope in situ hybridization of Ccl2 mRNA in TG of wild-type mice after repeated vehicle or NTG administration in accordance with the present disclosure. FIG. 8A includes representative images of two Ccl2⁺ non-neuronal cells in a fiber-rich area of mouse TG. FIG. 8B includes representative images of Ccl2⁺ cells in TG sections from mice that received repeated vehicle or NTG administration. FIG. 8C is a bar graph showing the mean intensity of Ccl2 signals in TG from female C57BL/6J mice that received i.p. vehicle or NTG injections every 2 days for 5 times (n=3). TG tissues were collected two days after the last injection.

FIG. 9 is an exemplary embodiment showing RFP⁺, CCL2-expressing cells in TG and dura from CCL2-RFP mice in accordance with the present disclosure. FIG. 9 is a line graph showing facial mechanical responses of male CCL2-RFP mice to repeated vehicle or NTG administration (n=6). Two-way RM ANOVA: P<0.001 for group (F_{[1, 60]}=28.10), time (F_{[5, 60]}=11.06), and group×time interaction (F_{[5, 60]}=7.14); post-hoc Student-Newman-Keuls test: ***P<0.001 between the corresponding Veh and NTG groups; ^{###}P<0.001, compared with the baseline (day 1) threshold within the NTG group.

FIG. 10 is an exemplary embodiment showing identification of CCL2- and CCR2-expressing cells in mouse TG and dura in accordance with the present disclosure. FIG. 10 is a bar graph showing the percentage of RFP⁺ neurons in TG cultures from vehicle- and NTG-treated mice (n=4 mice/group; a total of 258 and 274 neurons were measured in veh and NTG groups, respectively).

FIG. 11A-FIG. 11C is an exemplary embodiment showing RFP⁺, CCL2-expressing cells in TG and dura from CCL2-RFP mice in accordance with the present disclosure. FIG. 11A is a bar graph showing the percentage of medium-sized neurons (diameter >25 μm) in TG cultures from vehicle and NTG-treated mice (n=4 mice/group; a total of 258 and 274 neurons were measured in Veh and NTG groups, respectively). FIG. 11B includes Venn diagrams of the percentages of RFP⁺, IB4⁺, and capsaicin-responsive (Cap-R) neurons in TG culture from CCL2-RFP mice (same neurons as in FIG. 11A, data from vehicle- and NTG-treated mice are combined). FIG. 11C includes Venn diagrams of the percentages of RFP⁺, CGRP-R, and PACAP-R neurons in TG culture from CCL2-RFP mice (same neurons as in FIG. 11A, data from vehicle- and NTG-treated mice are combined).

FIG. 12 is an exemplary embodiment showing identification of CCL2- and CCR2-expressing cells in mouse TG and dura in accordance with the present disclosure. FIG. 12 includes representative images of RFP-immunoreactivity (RFP-ir) and CD31-immunoreactivity (CD31-ir) in whole-mount dura from CCL2-RFP mice.

FIG. 13 is an exemplary embodiment showing RFP⁺, CCL2-expressing cells in TG and dura from CCL2-RFP mice in accordance with the present disclosure. FIG. 13 includes representative images of RFP-ir and CD31-ir in whole-mount dura from CCL2-RFP mice.

FIG. 14 is an exemplary embodiment showing identification of CCR2-expressing cells in mouse TG and dura in accordance with the present disclosure. FIG. 14 includes representative images of Ccr2⁺ cells in a mouse TG section.

FIG. 15A-FIG. 15C is an exemplary embodiment showing RNAscope in situ hybridization of Ccr2 mRNA in TG of wild-type mice after repeated vehicle or NTG administration in accordance with the present disclosure. FIG. 15A includes representative images of Ccr2⁺ non-neuronal cells in a fiber-rich area of mouse TG. FIG. 15B includes representative images of Ccr2⁺ cells in TG sections from mice that received repeated vehicle or NTG administration. FIG. 15C is a bar graph showing the mean intensity of Ccr2 signals in TG from female C57BL/6J mice that received repeated vehicle or NTG administration (same mice as in FIG. 1C).

FIG. 16A-FIG. 16B is an exemplary embodiment showing identification of CCL2- and CCR2-expressing cells in mouse TG and dura in accordance with the present disclosure. FIG. 16A includes representative images of EGFP-ir and Iba1-ir in dura and TG tissues from Ccr2 HZ mice. Arrowheads indicate EGFP⁺Iba1⁺ macrophages. FIG. 16B includes representative images of EGFP-ir and CD3-ir in dura and TG tissues from Ccr2 HZ mice. Arrowheads indicate EGFP⁺CD3⁺ T cells.

FIG. 17A-FIG. 17H is an exemplary embodiment showing quantification of macrophages and T cells in the dura and TG of female Ccr2 HZ and KO mice in accordance with the present disclosure. FIG. 17A includes representative images of EGFP-ir and Iba1-ir in dura and TG tissues from Ccr2 HZ and KO mice. Arrowheads indicate EGFP⁺Iba1⁺ macrophages. FIG. 17B and FIG. 17C are bar graphs showing the densities of EGFP⁺Iba1⁺ (FIG. 17B) and Iba1⁺ (FIG. 17C) macrophages in the non-perivascular region of the dura (surrounding the MMA but not adjacent to the blood vessels) of female Ccr2 HZ and KO mice (n=8-9 mice/group). FIG. 17D and FIG. 17E are bar graphs showing the densities of EGFP⁺Iba1⁺ (FIG. 17D) and Iba1⁺ (FIG. 17E) cells in the perivascular region of the dura (same mice as in FIG. 17B and FIG. 17C). *P<0.05, two-tailed t-test. FIG. 17F-FIG. 17H include bar graphs showing the densities of EGFP⁺Iba1⁺ (FIG. 17F) and Iba1⁺ (FIG. 17G) macrophages as well as the ratio of EGFP⁺ cells to Iba1⁺ macrophages (FIG. 17H) in the TG of female Ccr2 HZ and KO mice (same mice as in FIG. 17B and FIG. 17C). *P<0.05, two-tailed t-test.

FIG. 18A-FIG. 18C is an exemplary embodiment showing identification of CCL2- and CCR2-expressing cells in mouse TG and dura in accordance with the present disclosure. FIG. 18A-FIG. 18C include bar graphs showing the densities of EGFP⁺Iba1⁺ macrophages in the non-perivascular region (FIG. 18A) and perivascular region of the dura (FIG. 18B) as well as in TG tissues (FIG. 18C) from female Ccr2 HZ and KO mice (n=8-9 mice/group). *P<0.05, two-tailed t-test.

FIG. 19A-FIG. 19F is an exemplary embodiment showing quantification of macrophages and T cells in the dura and TG of female Ccr2 HZ and KO mice in accordance with the present disclosure. FIG. 19A-FIG. 19C include bar graphs showing the density of EGFP⁺CD3⁺ (FIG. 19A) and CD3⁺ (FIG. 19B) T cells as well as the abundance of EGFP⁺ cells in the CD3⁺ population (FIG. 19C) in the dura of female Ccr2 HZ and KO mice (same mice as in FIG. 17B and FIG. 17C). FIG. 19D-FIG. 19F include bar graphs showing the number of EGFP⁺CD3⁺ (FIG. 19D) and CD3⁺ (FIG. 19E) T cells as well as the ratio of EGFP⁺ cells to CD3⁺ cells (FIG. 19F) in TG of female Ccr2 HZ and KO mice (same mice as in FIG. 17B and FIG. 17C).

FIG. 20A-FIG. 20B is an exemplary embodiment showing identification of CCL2- and CCR2-expressing cells in mouse TG and dura in accordance with the present disclosure. FIG. 20A-FIG. 20B include bar graphs showing the number of EGFP⁺CD3⁺ T cells in the dura (FIG. 20A) and TG (FIG. 20B) of female Ccr2 HZ and KO mice (n=4-8 mice/group).

FIG. 21A-FIG. 21B is an exemplary embodiment showing quantification of macrophages and T cells in the dura and TG of female Ccr2 HZ and KO mice in accordance with the present disclosure. FIG. 21A-FIG. 21B include bar graphs showing the density of EGFP⁺CD25⁺ cells in the dura (FIG. 21A) and the number of EGFP⁺CD25⁺ cells in TG (FIG. 21B) of female Ccr2 HZ and KO mice (same mice as in FIG. 17B and FIG. 17C).

FIG. 22A-FIG. 22D is an exemplary embodiment showing flow cytometry verification of the specificity of Ccr2 deletion in the peripheral blood of CD4CreCcr2^{fl/fl} and LysMCreCcr2^{fl/fl} mice in accordance with the present disclosure. FIG. 22A-FIG. 22B include graphs showing representative flow cytometry analysis of T cells (FIG. 22A) and monocytes (FIG. 22B) in peripheral blood cells isolated from Ccr2^{fl/fl}, CD4CreCcr2^{fl/fl} and LysMCreCcr2^{fl/fl} mice. FIG. 22C-FIG. 22D include bar graphs showing the frequency of EGFP⁺, Ccr2-deficient cells among CD3⁺ T cells (FIG. 22C) and CD11 b⁺ monocytes (FIG. 22D) in the peripheral blood cells from Ccr2^{fl/fl}, CD4CreCcr2^{fl/fl} and LysMCreCcr2^{fl/fl} mice (n=3-4/group).

FIG. 23A-FIG. 23G is an exemplary embodiment showing CD4CreCcr2^{fl/fl} and LysMCreCcr2^{fl/fl} mice do not develop NTG-induced acute or persistent behavioral sensitization in accordance with the present disclosure. FIG. 23A includes representative images of EGFP⁺, Ccr2-deficient cells in the population of CD3⁺ T cells and Iba1⁺ macrophages in the dura and TG of Ccr2^{fl/fl}, CD4CreCcr2^{fl/fl}, LysMCreCcr2^{fl/fl} or AvCreCcr2^{fl/fl} mice. Arrowheads indicate EGFP⁺CD3⁺ T cells or EGFP⁺Iba1⁺ macrophages. FIG. 23B is a bar graph showing facial mechanical thresholds of female Ccr2^{fl/fl}, LysMCreCcr2^{fl/fl}, and CD4CreCcr2^{fl/fl} mice 2 hours after the first NTG injection (n=5 mice/group). ***<0.001, one-way ANOVA with post hoc Bonferroni test. FIG. 23C is a line graph showing repeated NTG administration does not induce persistent facial mechanical hypersensitivity or hyperalgesic priming in female LysMCreCcr2^{fl/fl} and CD4CreCcr2^{fl/fl} mice (same mice as in FIG. 23B). Day 1-20, two-way RM ANOVA: P<0.001 for group (F_{[2, 120]}=60.96), time (F_{[8, 120]}=9.35), and group×time interaction (F_{[16, 120]}=7.26); post-hoc Student-Newman-Keuls test: ***P<0.001, Ccr2^{fl/fl} versus CD4CreCcr2^{fl/fl} or LysMCreCcr2^{fl/fl} groups; ^{#}P<0.05, ^{###}P<0.001, compared with the baseline threshold (day 1) in the Ccr2^{fl/fl} group. Day 20-25, two-way RM ANOVA: P<0.001 for group (F_{[2, 30]}=4.74), time (F_{[2, 30]}=40.72), and group×time interaction (F_{[4, 30]}=19.80); post-hoc Student-Newman-Keuls test: ***P<0.001, Ccr2^{fl/fl} versus CD4CreCcr2^{fl/fl} or LysMCreCcr2^{fl/fl} groups; ^{###}P<0.001, compared with the day 20 threshold in the Ccr2^{fl/fl} group. FIG. 23D is a line graph showing repeated NTG does not cause behavioral sensitization in male LysMCreCcr2^{fl/fl} and CD4CreCcr2^{fl/fl} mice (n=5-6 mice/group). Day 1-20, two-way RM ANOVA: P<0.001 for group (F_{[2, 128]}=8.06), time (F_{[8, 128]}=12.02), and group×time interaction (F_{[16, 128]}=6.05); post-hoc Student-Newman-Keuls test: *P<0.05, **P<0.01, Ccr2^{fl/fl} versus CD4CreCcr2^{fl/fl} or LysMCreCcr2^{fl/fl} groups; ^{###}P<0.001, compared with the day 1 threshold in the Ccr2^{fl/fl} group. Day 20-25, two-way RM ANOVA: P<0.001 for group (F_{[2, 32]}=2.82), time (F_{[2, 32]}=16.00), and group×time interaction (F_{[4, 32]}=4.67); post-hoc Student-Newman-Keuls test: ***P<0.001, Ccr2^{fl/fl} versus CD4CreCcr2^{fl/fl} or LysMCreCcr2^{fl/fl} groups; ^{###}P<0.001, compared with the day 20 threshold in the Ccr2^{fl/fl} group. FIG. 23E is a bar graph showing facial mechanical thresholds of female Ccr2^{fl/fl} and AvCreCcr2^{fl/fl} mice 2 hours after the first NTG injection (n=6-7 mice/group). FIG. 23F is a line graph showing the effects of repeated NTG administration on the 50% withdrawal thresholds to von Frey filaments at the periorbital region of female Ccr2^{fl/fl} and AvCreCcr2^{fl/fl} mice (same mice as in FIG. 23E). Day 1-20, one-way RM ANOVA: P<0.01 for CCR2^{fl/fl} (F_{[8, 48]}=26.78) and AvCreCcr2^{fl/fl} (F_{[8, 56]}=23.63) groups; post-hoc Student-Newman-Keuls test: ⁺⁺P<0.01, ⁺⁺⁺P<0.001, compared with the day 1 threshold in individual groups. Day 20-25, one-way RM ANOVA: P<0.01 for Ccr2^{fl/fl} (F_{[2, 12]}=7.01) and AvCreCCR2^{fl/fl} (F_{[2, 14]}=5.65) groups; post-hoc Student-Newman-Keuls test: ⁺P<0.05, compared with the day 20 threshold in individual groups. FIG. 23G is a line graph showing the effects of repeated NTG administration on the 50% withdrawal thresholds to von Frey filaments at the periorbital region of male Ccr2^{fl/fl} and AvCreCcr2^{fl/fl} mice (n=5-8 mice/group). Day 1-20, one-way RM ANOVA: P<0.01 for Ccr2^{fl/fl} (F_{[6, 64]}=17.93) and AvCreCcr2^{fl/fl} (F_{[8, 40]}=17.83) groups; post-hoc Student-Newman-Keuls test: ⁺⁺⁺P<0.001, compared with the day 1 threshold in individual groups. Day 20-25, one-way RM ANOVA: P<0.01 for Ccr2^{fl/fl} (F_{[2, 16]}=15.77) and AvCreCcr2^{fl/fl} (F_{[2, 10]}=17.59) groups; post-hoc Student-Newman-Keuls test: ⁺⁺⁺P<0.001, compared with the day 20 threshold in individual groups.

FIG. 24 is an exemplary embodiment showing neutralization of peripheral CCL2 blocks the development of repetitive stress-induced behavioral sensitization in accordance with the present disclosure. FIG. 24 is a line graph showing re-treatment with CCL2 ab prevents the development of repetitive stress-induced behavioral sensitization and hyperalgesic priming (n=3-6 male CD-1 mice). Day 1-14, two-way RM ANOVA: P<0.001 for group (F_{[2, 75]}=28.47), time (F_{[5, 75]}=15.28), and group×time interaction (F_{[10, 75]}=8.41); post-hoc Student-Newman-Keuls test: ***P<0.001, stress+control IgG versus sham or stress+CCL2 ab groups; ^{###}P<0.001, compared with the baseline threshold in the stress+control IgG group. Day 14-21, two-way RM ANOVA: P<0.001 for group (F_{[2, 30]}=2.37), time (F_{[2, 30]}=21.81), and group×time interaction (F_{[4, 30]}=12.29); post-hoc student-Newman-Keuls test: ***P<0.001, stress+control IgG versus sham or stress+CCL2 ab groups; ^{###}P<0.001, compared with the day 14 threshold in the stress+control IgG group.

FIG. 25A-FIG. 25B is an exemplary embodiment showing repetitive stress does not change Ccl2 or Ccr2 mRNA levels in TG or dura in accordance with the present disclosure. FIG. 25A-FIG. 25B include bar graphs showing relative Ccl2 (FIG. 25A) and Ccr2 (FIG. 25B) mRNA expression levels in the dura and TG from male CD-1 mice 1 day after the last restraint stress session (n=5-6 mice/group). The abundance of Ccl2 and Ccr2 mRNA was normalized to that of GAPDH in individual samples.

FIG. 26A-FIG. 26C is an exemplary embodiment showing neutralization of peripheral CCL2 blocks the development of repetitive stress-induced behavioral sensitization in accordance with the present disclosure. FIG. 26A is a line graph showing delayed CCL2 ab treatment reverses repetitive stress-induced behavioral sensitization and prevents hyperalgesic priming (n=6 male CD-1 mice/group). Day 1-14, two-way RM ANOVA: P<0.001 for group (F_{[1, 60]}=30.23), time (F_{[5, 60]}=27.03), and group×time interaction (F_{[5, 60]}=10.04); post-hoc Student-Newman-Keuls test: ***P<0.001, stress+control IgG versus stress+CCL2 ab groups; ^{###}P<0.001, relative to the baseline threshold in the stress+control IgG group. ⁺⁺⁺P<0.001, relative to the baseline threshold in the stress+CCL2 ab group. Day 14-21, two-way RM ANOVA: P<0.001 for group (F_{[1, 24]}=2.26), time (F_{[2, 24]}=23.75), and group×time interaction (F_{[2, 24]}=4.25); post-hoc Student-Newman-Keuls test: *P<0.05, stress+control IgG versus stress+CCL2 ab groups; ^{###}P<0.001, compared with the day 14 threshold in the stress+control IgG group. FIG. 26B is a line graph showing CCL2 ab treatment blocks the expression of repetitive stress-induced hyperalgesic priming (n=3-6 female CD-1 mice/group). Day 1-14, two-way RM ANOVA: P<0.001 for group (F_{[2, 75]}=49.75), time (F_{[5, 75]}=37.97), and group×time interaction (F_{[10, 75]}=6.94); post-hoc Student-Newman-Keuls test: ***P<0.001, sham versus stress+control IgG or stress+CCL2 ab groups; ^{###}P<0.001, compared with the baseline threshold in the stress+control IgG or stress+CCL2 ab groups. Day 14-21, two-way RM ANOVA: P<0.001 for group (F_{[2, 30]}=29.32), time (F_{[2, 30]}=17.85), and group×time interaction (F_{[4, 30]}=20.48); post-hoc Student-Newman-Keuls test: ^{$$$}P<0.001, stress+control IgG versus sham or stress+CCL2 ab groups; ⁺⁺⁺P<0.001, compared with the day 14 threshold in the stress+control IgG group. FIG. 26C is a line graph showing delayed 11K2 antibody treatment reverses repetitive stress-induced behavioral sensitization and prevents hyperalgesic priming (n=3-6 male CD-1 mice). Day 1-14, two-way RM ANOVA: P<0.001 for group (F_{[2, 75]}=23.86), time (F_{[5, 75]}=15.76), and group×time interaction (F_{[10, 75]}=6.00); post-hoc Student-Newman-Keuls test: ***P<0.001, stress+11K2 versus stress+control IgG, ^{!!!}P<0.001, stress+11K2 versus sham; ^{∧∧∧}P<0.001, stress+control IgG versus sham; ^{###}P<0.001, compared with the baseline threshold in the stress+control IgG group; +++P<0.001, compared with the baseline threshold in the stress+11K2 group. Day 14-21, two-way RM ANOVA: P<0.001 for group (F_{[2, 30]}=6.390), time (F_{[2, 30]}=2.058), and group×time interaction (F_{[4, 30]}=1.510); post-hoc Student-Newman-Keuls test: ^{$$$}P<0.001, stress+control IgG versus stress+CCL2 ab or sham groups; ^{###}P<0.001, compared with the day 14 threshold in the stress+control IgG group.

FIG. 27A-FIG. 27B is an exemplary embodiment showing neutralization of peripheral CCL2 blocks the development of repetitive stress-induced behavioral sensitization in accordance with the present disclosure. FIG. 27A shows facial pain score criteria. FIG. 27B is a bar graph showing the effects of control IgG and anti-CCL2 antibody 11K2 on repetitive stress-induced increase in facial pain score (same mice as in FIG. 26C). For each experimental group, responses from all mice to individual von Frey filaments were combined to calculate the frequency of individual scores. ***P<0.001, χ² test followed by post hoc Fisher's exact test with Bonferroni correction.

FIG. 28A-FIG. 28C is an exemplary embodiment showing neutralization of peripheral CCL2 blocks the development of repetitive stress-induced behavioral sensitization in accordance with the present disclosure. FIG. 28A is a bar graph showing delayed 11K2 antibody treatment reduces stress-induced increase in facial pain score (same mice as in FIG. 26C). ***P<0.001, one-way ANOVA with post hoc Bonferroni test. FIG. 28B is a line graph showing pre-treatment with 11K2 antibody prevents the development of repeated NTG-induced behavioral sensitization and hyperalgesic priming (n=6 female C57BL/6J mice). Day 1-20, two-way RM ANOVA: P<0.001 for group (F_{[1, 96]}=50.58), time (F_{[8, 96]}=20.73), and group×time interaction (F_{[8, 96]}=6.03); post-hoc Student-Newman-Keuls test: ***P<0.001, 11K2 versus control IgG groups; ^{##}P<0.01, ^{###}P<0.001, compared with the baseline threshold in the control IgG group; ^{&}P<0.05, compared with the baseline threshold in the 11K2 group. Day 20-25, two-way RM ANOVA: P<0.001 for group (F_{[1, 24]}=22.48), time (F_{[2, 24]}=31.81), and group×time interaction (F_{[2, 24]}=15.51); post-hoc Student-Newman-Keuls test: ***P<0.001, 11K2 versus control IgG group; ^{###}P<0.001, compared with the day 20 threshold in the control IgG group. FIG. 28C is a bar graph showing re-treatment with the 11K2 antibody blocks NTG-induced increase in facial pain score (same mice as in FIG. 28B). **P<0.01, two-tailed t-test.

FIG. 29 is an exemplary embodiment showing neutralization of peripheral CCL2 blocks the development of repetitive stress-induced behavioral sensitization in accordance with the present disclosure. FIG. 29 is a bar graph showing the effects of control IgG and 11K2 antibody on repeated NTG-induced increase in facial pain score (same mice as in FIG. 28B). ***P<0.001, χ² test followed by post hoc Fisher's exact test with Bonferroni correction.

FIG. 30A-FIG. 30I is an exemplary embodiment showing repeated NTG administration increases the percentages of CGRP-R and PACAP-R TG neurons in wild-type mice but not in Ccl2 or Ccr2 global KO mice in accordance with the present disclosure. FIG. 30A-FIG. 30C include bar graphs (FIG. 30A-FIG. 30B) and Venn diagrams (FIG. 30C) indicating the percentages of CGRP-R and PACAP-R neurons in TG cultures from male wild-type and Ccl2 KO mice after 5 repeated vehicle or NTG injections. A total of 115, 108, 167 and 190 TG neurons were measured in wild-type+Veh, wild-type+NTG, Ccl2 KO+Veh and Ccl2 KO+NTG groups, respectively. **P<0.01,***P<0.001, χ² test followed by post hoc Fisher's exact test with Bonferroni correction. FIG. 30D-FIG. 30F include bar graphs (FIG. 30D-FIG. 30E) and Venn diagrams (FIG. 30F) indicating the percentages of CGRP-R and PACAP-R neurons in TG cultures from female wild-type and Ccl2 KO mice after 5 repeated vehicle or NTG injections. A total of 76, 71, 95 and 175 TG neurons were measured in wild-type+Veh, wild-type+NTG, Ccl2 KO+Veh and Ccl2 KO+NTG groups, respectively. *P<0.05, **P<0.01, χ² test followed by post hoc Fisher's exact test with Bonferroni correction. FIG. 30G-FIG. 30I include bar graphs (FIG. 30G-FIG. 30H) and Venn diagrams (FIG. 30I) indicating the percentages of CGRP-R and PACAP-R neurons in TG cultures from female wild-type and Ccr2 KO mice after 5 repeated vehicle or NTG injections. A total of 142, 189, 265 and 241 TG neurons were measured in wild-type+Veh, wild-type+NTG, Ccr2 KO+Veh and Ccr2 KO+NTG groups, respectively. *P<0.05, **P<0.01, χ² test followed by post hoc Fisher's exact test with Bonferroni correction.

FIG. 31A-FIG. 31D is an exemplary embodiment showing inhibition of both peripheral CCL2 and CGRP signaling is more effective in reversing NTG-induced sensitization than targeting individual pathways in accordance with the present disclosure. FIG. 31A is a bar graph showing CGRP concentrations in TG tissues from wild-type and CCR2 global KO mice after repeated vehicle or NTG administration (n=5-8 female mice/group). **P<0.01, two-way ANOVA with post hoc Bonferroni test. FIG. 31B is a schematic showing a summary of the contributions of peripheral CCL2-CCR2 and CGRP signaling pathways to NTG-induced sensitization. FIG. 31C is a line graph showing the effects of CCL2 ab and/or CGRP ab on NTG-induced persistent facial skin hypersensitivity. Arrows indicate the i.p. injection of NTG, control IgG (n=8 female C57BL/6J mice), 3 mg/kg CCL2 ab (n=9), 30 mg/kg CGRP ab (n=6), or co-administration of CCL2 ab and CGRP ab (CCL2ab+CGRPab, n=8). Mice in the control IgG group received 3 mg/kg control IgG for CCL2 ab and 30 mg/kg control IgG for CGRP ab. Day 1-20, two-way RM ANOVA: P<0.001 for group (F_{[3, 248]}=33.7), time (F_{[8, 248]}=112.5), and group×time interaction (F_{[24, 248]}=5.6); post-hoc Student-Newman-Keuls test: ***P<0.001, CCL2ab+CGRPab versus all other groups; ^{∧∧}P<0.01, ^{∧∧∧}P<0.001, compared with the baseline (day 1) threshold in the CCL2ab+CGRPab group; ^{##}P<0.01, ^{###}P<0.001, compared with the baseline (day 1) threshold within the control IgG, CCL2 ab and CGRP ab groups, respectively. FIG. 31D is a line graph showing the effects of CCL2 ab and/or CGRP ab on NTG-induced hyperalgesic priming (same mice as in FIG. 31A, except that n=4 in the CCL2 ab+CGRP ab group. The other 4 mice did not receive 0.1 mg/kg NTG injections). Day 20-25, two-way RM ANOVA: P<0.001 for group (F_{[3, 58]}=16.6), time (F_{[2, 58]}=383.9), and group×time interaction (F_{[6, 58]}=51.2); post-hoc Student-Newman-Keuls test: ***P<0.001, CCL2 ab+CGRP ab versus all other groups; ^{###}P<0.001, compared with the baseline (day 20) threshold within the control IgG, CCL2 ab and CGRP ab groups, respectively.

FIG. 32A-FIG. 32E is an exemplary embodiment showing Ccl2 and Ccr2 global knockout (KO) mice do not exhibit behaviors related to acute post-traumatic headache (PTH) but still retain chronic PTH-related hyperalgesic priming in accordance with the present disclosure. FIG. 32A is timeline of the experiments. Note that NTG was injected after the completion of behavioral tests on the same day. FIG. 32B and FIG. 32C are line graphs showing female Ccl2 and Ccr2 KO mice that received weight drop from 50 cm height did not exhibit mTBI-induced acute sensitization (FIG. 32B) but developed hyperalgesic priming (FIG. 32C). n=5-10/group. FIG. 32B: two-way RM ANOVA: P<0.001 for group (F_{[4, 280]}=31.22), time (F_{[7, 280]}=6.47), and group×time interaction (F_{[28, 280]}=3.98); post hoc Student-Newman-Keuls test: ***P<0.001, wild-type:mTBI versus all other groups; ^{###}P<0.001, compared with the baseline (day 0) threshold in the wild-type:mTBI group. FIG. 32C: two-way RM ANOVA: P<0.001 for group (F_{[4, 200]}=25.84), time (F_{[5, 200]}=35.95), and group×time interaction (F_{[20, 200]}=6.50); post hoc Student-Newman-Keuls test: ^{∧∧∧}P<0.001, wild-type:mTBI or Ccl2 KO:mTBI versus wild-type:sham or Ccl2 KO:sham groups on day 22, 37 and 51; ⁺⁺⁺P<0.001, day 22 versus 20, day 37 versus 35, and day 51 versus 49 within wild-type:mTBI, Ccl2 KO:mTBI and Ccr2 KO:mTBI groups. FIG. 32D and FIG. 32E are line graphs showing male Ccl2 and Ccr2 KO mice that received weight drop from 50 cm height did not exhibit mTBI-induced acute sensitization (FIG. 32D) but developed hyperalgesic priming (FIG. 32E). n=10-14/group. FIG. 32D: two-way RM ANOVA: P<0.001 for group (F_{[2, 245]}=0.55), time (F_{[5, 245]}=33.38), and group×time interaction (F_{[10, 245]}=0.72); post hoc Student-Newman-Keuls test: ***P<0.001, wild-type versus Ccl2 KO and Ccr2 KO groups; ^{###}P<0.001, compared with the baseline (day 0) threshold in the wild-type group. FIG. 32E: two-way RM ANOVA: P=0.582 for group (F_{[2, 175]}=0.55, no significant difference), P<0.001 for time (F_{[5, 175]}=33.38). One-way RM ANOVA: P<0.001 for wild-type (F_{[5, 55]}=6.47), Ccl2 KO (F_{[5, 70]}=5.76) and Ccr2 KO groups (F_{[5, 50]}=3.64); post hoc Student-Newman-Keuls test: ⁺⁺⁺P<0.001, day 22 versus 20, day 37 versus 35, and day 51 versus 49 within wild-type, Ccl2 KO, and Ccr2 KO groups.

FIG. 33A-FIG. 33E is an exemplary embodiment showing peripheral CCL2-CCR2 signaling is essential for the development and maintenance of acute PTH-related sensitization but not chronic PTH-related hyperalgesic priming in accordance with the present disclosure. FIG. 33A is a timeline of the experiments in male C57BL/6J mice. Note that NTG and CCL2 ab were injected after the completion of behavioral tests on the same day. FIG. 33B is a line graph showing starting the CCL2 ab treatment before mTBI prevented the development of acute facial skin hypersensitivity but not hyperalgesic priming (n=9-10/group). Day 1-20, two-way RM ANOVA: P<0.001 for group (F_{[1, 133]}=48.11), time (F_{[7, 133]}=20.02), and group×time interaction (F_{[7, 133]}=12.59); post hoc Student-Newman-Keuls test: ***P<0.001, between the corresponding control IgG and CCL2 ab groups; ^{##π}P<0.001, compared with the baseline (day 0) threshold in the control IgG group. Day 20-24, two-way RM ANOVA: P=0.499 for group (F_{[1, 38]}=0.48, no significant difference), P<0.001 for time (F_{[2, 38]}=91.70). One-way RM ANOVA: P<0.001 for control IgG (F_{[2, 18]}=45.40) and CCL2 ab groups (F_{[2, 20]}=53.65); post hoc Student-Newman-Keuls test: ⁺⁺⁺P<0.001, compared with the baseline (day 20) threshold in the control IgG and CCL2 ab groups, respectively. FIG. 33C is a line graph showing starting the CCL2 ab treatment 1 day post-mTBI protected mice from developing acute facial skin hypersensitivity but did not prevent hyperalgesic priming (n=8/group). Day 1-20, two-way RM ANOVA: P<0.001 for group (F_{[1, 112]}=26.37), time (F_{[7, 112]}=4.45), and group×time interaction (F_{[7, 112]}=3.68); post hoc Student-Newman-Keuls test: ***P<0.001, between the corresponding control IgG and CCL2 ab groups; ^{###}P<0.001, compared with the baseline (day 0) threshold in the control IgG group. Day 20-24, two-way RM ANOVA: P=0.851 for group (F_{[1, 32]}=0.04, no significant difference), P<0.001 for time (F_{[2, 32]}=35.89). One-way RM ANOVA: P<0.001 for control IgG (F_{[2, 16]}=13.67) and CCL2 ab groups (F_{[2, 16]}=15.90); post hoc Student-Newman-Keuls test: ⁺⁺⁺P<0.001, compared with the baseline (day 20) threshold in the control IgG and CCL2 ab groups, respectively. FIG. 33D is a line graph showing delayed CCL2 ab treatment accelerated the resolution of mTBI-induced acute facial skin hypersensitivity but did not prevent the development of hyperalgesic priming (n=9-11/group). Day 1-20, two-way RM ANOVA: P<0.001 for group (F_{[1, 140]}=11.02), time (F_{[7, 140]}=52.42), and group×time interaction (F_{[7, 140]}=4.04); post hoc Student-Newman-Keuls test: ***P<0.001, between the corresponding control IgG and CCL2 ab groups; ^{###}P<0.001, compared with the baseline (day 0) threshold in the control IgG group. ^{∧}P<0.05, ^{∧∧}P<0.01, ^{∧∧∧}P<0.001, compared with the day 0 threshold in CCL2 ab group. Day 20-24, two-way RM ANOVA: P=0.077 for group (F_{[1, 40]}=3.52, no significant difference), P<0.001 for time (F_{[2, 40]}=42.39). One-way RM ANOVA: P<0.001 for control IgG (F_{[2, 22]}=32.88) and CCL2 ab groups (F_{[2, 18]}=16.84); post hoc Student-Newman-Keuls test: ^{###}P<0.001, compared with the baseline (day 20) threshold in the control IgG group; ^{∧∧∧}P<0.001, compared with the day 20 threshold in the CCL2 ab group. FIG. 33E is a line graph showing treating mice with CCL2 ab after the resolution of acute facial skin hypersensitivity did not prevent the development or the expression of hyperalgesic priming (n=3-4/group).

FIG. 34 is a line graph showing inhibition of both peripheral CGRP and CCL2-CCR2 signaling blocks mTBI-induced hyperalgesic priming that is mechanistically related to chronic PTH. Day 0-35, acute sensitization phase, one-way RM ANOVA: P<0.001; post hoc Student-Newman-Keuls test: ^{##}P<0.01, ^{###}P<0.001, compared with the baseline threshold on day 0. Day 35-45, hyperalgesic priming phase, one-way RM ANOVA: P>0.05, no significant difference relative to the baseline threshold on day 35.

### DETAILED DESCRIPTION

The present disclosure is based, at least in part, on the discovery that peripheral CCL2 and CCR2 are potential targets for migraine and post-traumatic headache. As shown herein, the peripheral CCL2-CCR2 signaling pathway contributes to migraine chronification. The present disclosure provides for targeting peripheral CCL2-CCR2 signaling to treat migraine and post-traumatic headache.

Headache disorders, including migraine and post-traumatic headache (PTH), are highly prevalent, poorly understood and extremely debilitating. A substantial proportion of patients remains either unresponsive to current treatment options or intolerant to their side effects. Overuse of anti-migraine drugs and opioids leads to medication overuse headache, further exacerbating the condition. There is a pressing need to develop safer and more effective therapies with mechanisms of action distinct from the existing approaches.

Antagonists and blocking antibodies against chemokine C—C motif ligand 2 (CCL2, MCP1) and C—C chemokine receptor type 2 (CCR2) are well tolerated in humans but their effectiveness against migraine and post-traumatic headache (PTH) has not previously been tested.

As described herein, both CCL2 neutralizing antibody and CCR2 antagonist effectively reverse headache-related behavioral sensitization in mouse models of chronic migraine and post-traumatic headache (PTH). Coadministration of sub-effective doses of antibodies against CCL2 and CGRP (calcitonin gene-related peptide, FDA-approved for migraine prevention) completely reverses chronic migraine-related behaviors in mice.

Collectively, peripheral CCL2 and CCR2 are identified herein as potential targets for migraine and PTH therapy. Importantly, the results suggest that inhibition of both peripheral CGRP and CCL2-CCR2 signaling may be more effective in treating migraine and PTH than blocking either pathway alone.

CCL2-CCR2 Signaling Inhibiting Agent

One aspect of the present disclosure provides for targeting of CCL2, its receptor CCR2, or its downstream signaling. The present disclosure provides methods of treating or preventing headache or a headache disorder based on the discovery that the peripheral CCL2-CCR2 signaling pathway contributes to migraine chronification.

As described herein, inhibitors or antagonists of CCL2-CCR2 signaling (e.g., antibodies, fusion proteins, small molecules) can reduce or prevent migraine chronification and are an effective treatment for headache disorder. A CCL2-CCR2 signaling inhibiting agent can be any agent that can inhibit CCL2 or CCR2, downregulate CCL2 or CCR2, or knock down CCL2 or CCR2.

For example, the CCL2-CCR2 signaling inhibiting agent can be an anti-CCL2 antibody or an anti-CCR2 antibody. Furthermore, the anti-CCL2 antibody or the anti-CCR2 can be a murine antibody, a humanized murine antibody, or a human antibody. The anti-CCL2 antibody or anti-CCR2 antibody can be a neutralizing antibody, wherein the neutralizing antibody binds to CCL2 or CCR2 and inhibits, reduces, or eliminates its activity or signaling. For example, the anti-CCL2 antibody can be 11K2, carlumab, or MLN1202.

In some embodiments, the CCL2-CCR2 signaling inhibiting agent is a bispecific antibody. For example, the bispecific antibody may simultaneously bind and inhibit CCL2, CCR2, CGRP, CGRP receptor, or any combination thereof.

As another example, the CCL2-CCR2 signaling inhibiting agent can be a fusion protein. For example, the fusion protein can be a decoy receptor for CCL2.

As another example, a CCL2-CCR2 signaling inhibiting agent can be a small molecule antagonist of CCR2 or CCL2. As an example, the small molecule antagonist can be RS504393, BMS-741672, PF-04136309, or CNTX-6970, which have been shown to be potent and specific inhibitors of CCL2-CCR2 signaling.

As another example, a CCL2-CCR2 signaling inhibiting agent can be an inhibitory protein that antagonizes CCL2 or CCR2. For example, the CCL2-CCR2 signaling inhibiting agent can be a viral protein that antagonizes CCL2 or CCR2.

As another example, a CCL2-CCR2 signaling inhibiting agent can be a short hairpin RNA (shRNA) or a short interfering RNA (siRNA) targeting CCL2 or CCR2.

As another example, a CCL2-CCR2 signaling inhibiting agent can be a single guide RNA (sgRNA) targeting CCL2 or CCR2.

Inhibiting CCL2-CCR2 signaling can be performed by genetically modifying CCL2 or CCR2 in a subject or genetically modifying a subject to reduce or prevent expression of the CCL2 or CCR2 gene, such as through the use of CRISPR-Cas9 or analogous technologies, wherein, such modification reduces or prevents CCL2-CCR2 signaling.

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as ECK for excitatory drugs. ECK represents the dose or plasma concentration required for obtaining 50% of a maximum effect in vivo. IC₅₀ can be determined with functional assays or with competition binding assays.

CGRP Inhibiting Agent

One aspect of the present disclosure provides for targeting of CGRP or its downstream signaling. The present disclosure provides methods of treating or preventing headache or a headache disorder based on the discovery that inhibition of both CGRP and CCL2-CCR2 signaling is more effective in reversing chronic headache-related behavioral sensitization than targeting individual pathways alone.

As described herein, inhibitors or antagonists of CGRP (e.g., antibodies, fusion proteins, small molecules) are particularly effective in combination with CCL2-CCR2 signaling inhibiting agents for treating, preventing, or reversing headache or headache disorders. A CGRP inhibiting agent can be any agent that can inhibit CGRP, downregulate CGRP, or knock down CGRP.

For example, the CGRP inhibiting agent can be an anti-CGRP antibody or an antibody against the CGRP receptor. Furthermore, the anti-CGRP antibody or antibody against the CGRP receptor can be a murine antibody, a humanized murine antibody, or a human antibody. The anti-CGRP antibody or antibody against the CGRP receptor can be a neutralizing antibody, wherein the neutralizing antibody binds to CGRP or its receptor and inhibits, reduces, or eliminates its activity or signaling. For example, the anti-CGRP antibody or antibody against the CGRP receptor can be ALD405, Erenumab (Aimovig), Fremanezumab (Ajovy), Galcanezumab (Emgality), or Eptinezumab (Vyepti).

As another example, the CGRP inhibiting agent can be a fusion protein. For example, the fusion protein can be a decoy receptor for CGRP.

As another example, a CGRP inhibiting agent can be a small molecule antagonist of CGRP or its receptor. As an example, the small molecule antagonist can be a gepant, such as olcegepant, telcagepant, rimegepant, ubrogepant, atogepant, zavegepant, MK-3207, or BI 44370 which has been shown to be a potent and specific inhibitor of CGRP and/or its receptor.

As another example, a CGRP inhibiting agent can be an inhibitory protein that antagonizes CGRP or its receptor. For example, the CGRP inhibiting agent can be a viral protein that antagonizes CGRP or its receptor.

As another example, a CGRP inhibiting agent can be a short hairpin RNA (shRNA) or a short interfering RNA (siRNA) targeting CGRP or its receptor.

As another example, a CGRP inhibiting agent can be a single guide RNA (sgRNA) targeting CGRP or its receptor.

Inhibiting CGRP can be performed by genetically modifying CGRP or its receptor in a subject or genetically modifying a subject to reduce or prevent expression of the CGRP gene or that of its receptor, such as through the use of CRISPR-Cas9 or analogous technologies, wherein, such modification reduces or prevents CGRP signaling.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| | | Bases | Complementary |
|---|---|---|---|
| Base | Name | Represented | Base |
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:

(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.

(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| | Leu, Val, Met, Ala, |
| Ile (I) | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Genome Editing

As described herein, CCL2-CCR2 signaling or CGRP can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of CCL2 or CCR2 by genome editing can result in protection from headache or headache disorders.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for headache or headache disorders to target cells by the removal or addition of CCL2 or CCR2 signals (e.g., activate (e.g., CRISPRa), upregulate, overexpress, downregulate CCL2 or CCR2).

For example, the methods as described herein can comprise a method for altering a target polynucleotide sequence in a cell comprising contacting the polynucleotide sequence with a clustered regularly interspaced short palindromic repeats-associated (Cas) protein.

Gene Therapy and Genome Editing

Gene therapies can include inserting a functional gene with a viral vector. Gene therapies for headache and headache disorders are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses transcribing |
| | their single-stranded genome into a double- |
| | stranded DNA copy, which can integrate into |
| | host chromosome |
| Adenoviruses | Ad can transfect a variety of quiescent and |
| (Ad) | proliferating cell types from various species |
| | and can mediate robust gene expression |
| Adeno-associated | Recombinant AAV vectors contain no viral DNA |
| Viruses (AAV) | and can carry ~4.7 kb of foreign transgenic |
| | material. They are replication defective and |
| | can replicate only while coinfecting with a |
| | helper virus |
| Non-viral vectors | |
| plasmid DNA | pDNA has many desired characteristics as a |
| (pDNA) | gene therapy vector; there are no limits on |
| | the size or genetic constitution of DNA, it |
| | is relatively inexpensive to supply, and |
| | unlike viruses, antibodies are not generated |
| | against DNA in normal individuals |
| RNAi | RNAi is a powerful tool for gene specific |
| | silencing that could be useful as an enzyme |
| | reduction therapy or means to promote read- |
| | through of a premature stop codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating, preventing, or reversing a headache or headache disorder in a subject in need of administration of a therapeutically effective amount of a CCL2-CCR2 signaling inhibiting agent, alone or in combination with a CGRP inhibiting agent, so as to reduce, prevent, or reverse, for example, facial mechanical hyper-sensitivity or hyperalgesic priming, reduce the number of CGRP- and/or PACAP-responsive trigeminal ganglion (TG) neurons, or reduce or prevent sensitization of primary afferent neurons and the chronification of migraine headache.

Headache Disorders

A headache disorder as used herein refers to any condition characterized by headache. As described herein, headache disorders may be highly debilitating and lack effective treatment. The headache associated with a headache disorder may be a recurrent or chronic headache, occurring multiple days per month, for example. A headache disorder may be, but is not limited to, migraine, chronic migraine, chronic headache, stress-induced headache, acute post-traumatic headache (PTH), chronic PTH, headache resulting from traumatic brain injury (TBI) or mild traumatic brain injury (mTBI), chronic daily headache (CDH), cluster headache, hemicrania continua, idiopathic intracranial hypotension, tension-type headache, medication overuse headache, transformed migraine, paroxysmal hemicranias, primary stabbing headache, hypnic headache, or Valsalva-induced headache.

Methods described herein are generally performed on a subject in need thereof. A subject in need of the therapeutic methods described herein can be a subject having, diagnosed with, suspected of having, or at risk for developing a headache or headache disorder. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of CCL2-CCR2 signaling inhibiting agent, alone or in combination with a CGRP inhibiting agent, is, for example, an amount that would cause the desired therapeutic effect in a subject while minimizing undesired side effects. In various embodiments, an effective amount of CCL2-CCR2 signaling inhibiting agent, alone or in combination with a CGRP inhibiting agent, described herein can substantially reduce, prevent, or reverse facial mechanical hyper-sensitivity or hyperalgesic priming or reduce the number of CGRP- and/or PACAP-responsive trigeminal ganglion (TG) neurons or reduce or prevent sensitization of primary afferent neurons and the chronification of migraine headache.

In some embodiments, a “sub-effective” dose of a CCL2-CCR2 signaling inhibiting agent or a CGRP inhibiting agent may be administered to a subject. A sub-effective dose refers to a dose of the agent that is lower than the amount that is effective when the agent is administered alone. For example, a sub-effective dose of a CCL2-CCR2 signaling inhibiting agent administered in combination with a CGRP inhibiting agent, a sub-effective dose of a CGRP inhibiting agent administered in combination with a CCL2-CCR2 signaling inhibiting agent, or a sub-effective dose of a CCL2-CCR2 signaling inhibiting agent administered in combination with a sub-effective dose of a CGRP inhibiting agent may be used to reduce, prevent, or reverse symptoms of a headache or headache disorder in a subject, such as mechanical hypersensitivity or hyperalgesic priming.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a CCL2-CCR2 signaling inhibiting agent, alone or in combination with a CGRP inhibiting agent, can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce, prevent, or reverse facial mechanical hyper-sensitivity or hyperalgesic priming or reduce the number of CGRP- and/or PACAP-responsive trigeminal ganglion (TG) neurons or reduce or prevent sensitization of primary afferent neurons and the chronification of migraine headache.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Shamel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a CCL2-CCR2 signaling inhibiting agent, alone or in combination with a CGRP inhibiting agent, can occur as a single event or over a time course of treatment. For example, a CCL2-CCR2 signaling inhibiting agent, alone or in combination with a CGRP inhibiting agent, can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for headache or headache disorder.

A CCL2-CCR2 signaling inhibiting agent, alone or in combination with a CGRP inhibiting agent, can be administered simultaneously or sequentially with another agent, such as an antibiotic, an anti-inflammatory, or another agent. For example, a CCL2-CCR2 signaling inhibiting agent can be administered simultaneously with another agent, such as an antibiotic or an anti-inflammatory. Simultaneous administration can occur through administration of separate compositions, each containing one or more of a CCL2-CCR2 signaling inhibiting agent, an antibiotic, an anti-inflammatory, or another agent. Simultaneous administration can occur through administration of one composition containing two or more of a CCL2-CCR2 signaling inhibiting agent, an antibiotic, an anti-inflammatory, or another agent. A CCL2-CCR2 signaling inhibiting agent can be administered sequentially with an antibiotic, an anti-inflammatory, or another agent. For example, a CCL2-CCR2 signaling inhibiting agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, a CCL2-CCR2 signaling inhibiting agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a CCL2-CCR2 signaling inhibiting agent as described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to CCL2-CCR2 signaling inhibiting agents or CGRP inhibiting agents. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Peripheral CCL2-CCR2 Signaling Contributes to Chronic Headache-Related Sensitization

This example describes identification of peripheral CCL2 and CCR2 as potential targets for chronic migraine therapy and how inhibition of both peripheral CGRP and CCL2-CCR2 signaling is more effective for chronic migraine than targeting either pathway alone.

Migraine, especially chronic migraine, is highly debilitating and still lacks effective treatment. The persistent headache arises from activation and sensitization of primary afferent neurons in the trigeminovascular pathway, but the underlying mechanisms remain incompletely understood. Animal studies indicate that signaling through chemokine C—C motif ligand 2 (CCL2) and C—C motif chemokine receptor 2 (CCR2) mediates the development of chronic pain after tissue or nerve injury. Some migraine patients had elevated CCL2 levels in CSF or cranial periosteum. However, whether the CCL2-CCR2 signaling pathway contributes to chronic migraine is not clear.

Herein is described the modeling of chronic headache with repeated administration of nitroglycerin (NTG, a reliable migraine trigger in migraineurs). It was found that both Ccl2 and Ccr2 mRNA were upregulated in dura and trigeminal ganglion (TG) tissues that are implicated in migraine pathophysiology. In Ccl2 and Ccr2 global knockout mice, repeated NTG administration did not evoke acute or persistent facial skin hypersensitivity as in wild-type mice. Intraperitoneal injection of CCL2 neutralizing antibodies inhibited chronic headache-related behaviors induced by repeated NTG administration and repetitive restraint stress, suggesting that the peripheral CCL2-CCR2 signaling mediates headache chronification. CCL2 was mainly expressed in TG neurons and cells associated with dura blood vessels, whereas CCR2 was expressed in subsets of macrophages and T cells in TG and dura but not in TG neurons under both control and disease states. Deletion of Ccr2 gene in primary afferent neurons did not alter NTG-induced sensitization, but eliminating CCR2 expression in either T cells or myeloid cells abolished NTG-induced behaviors, indicating that both CCL2-CCR2 signaling in T cells and macrophages are required to establish chronic headache-related sensitization. At cellular level, repeated NTG administration increased the number of TG neurons that responded to calcitonin-gene-related peptide (CGRP) and pituitary adenylate cyclase-activating polypeptide (PACAP) as well as the production of CGRP in wild-type but not Ccr2 global knockout mice. Lastly, co-administration of CCL2 and CGRP neutralizing antibodies was more effective in reversing NTG-induced behaviors than individual antibodies.

Taken together, these results suggest that migraine triggers activate CCL2-CCR2 signaling in macrophages and T cells. This consequently enhances both CGRP and PACAP signaling in TG neurons, ultimately leading to persistent neuronal sensitization underlying chronic headache. This work not only identifies the peripheral CCL2 and CCR2 as potential targets for chronic migraine therapy, but also provides proof-of-concept that inhibition of both peripheral CGRP and CCL2-CCR2 signaling is more effective than targeting either pathway alone.

Introduction

Migraine is one of the most disabling diseases worldwide. Episodic migraine can evolve into chronic migraine, resulting in high frequency headache that severely compromises quality of life. Despite the recent advances of preventive therapies targeting the calcitonin-gene-related peptide (CGRP) pathway, many patients still do not benefit from currently available treatment options. There is an urgent need to better understand the disease mechanisms and to identify novel therapeutic targets.

Neuroimmune interactions via cytokine/chemokine signaling has long been recognized in many chronic pain conditions, including migraine. The level of chemokine C—C motif ligand 2 (CCL2, also called MCP-1, monocyte chemotactic protein-1) is increased in the CSF of migraine patients during attacks. Elevated CCL2 mRNA expression is found in the cranial periosteum of chronic migraine patients, and is identified by the interactive network analysis as a key driver in the inflammatory pathophysiology of the periosteum. However, whether the CCL2 signaling pathway functionally contributes to chronic headache remains unclear.

CCL2 is produced and secreted by many cells, including immune cells, glia, and primary afferent neurons in the dorsal root ganglion (DRG) and trigeminal ganglion (TG). Although CCL2 can activate multiple receptors, it exerts biological functions predominantly through C—C motif chemokine receptor 2 (CCR2) expressed in many types of cells. CCL2 also has the highest binding affinity to CCR2 among all chemokines that can activate the receptor. In various animal models, tissue or nerve injury increases CCL2 and CCR2 expression in peripheral and central neurons, satellite cells, astrocytes as well as microglia and other immune cells. Intrathecal or intraplantar administration of exogenous CCL2 in rodents rapidly produces heat and mechanical hypersensitivity. Genetic or pharmacological inhibition of CCL2-CCR2 signaling alleviates chronic pain-related behaviors in many animal models of tissue or nerve injury. Further mechanistic studies reveal that chronic pain can result from paracrine/autocrine CCL2-CCR2 signaling in primary afferent neurons or CCR2-mediated infiltration of monocytes/macrophages at peripheral. In spinal cord and cervical/medullary dorsal horn, CCL2-CCR2 signaling mediates both neuronal-to-microglial and astroglial-to-neuronal interactions that lead to neuropathic pain. Thus, activation of the CCL2-CCR2 signaling pathway contributes to tissue and nerve injury-induced chronic pain through multiple mechanisms.

Unlike injury-induced pain, migraine headache usually is not associated with actual tissue or neuronal damage, therefore is often described as a type of functional pain. In this study, it was investigated whether and how the CCL2-CCR2 signaling pathway contributes to migraine pathophysiology, especially the headache chronification. The induction of high frequency headache in chronic migraine patients was modeled with repeated administration of nitroglycerin (NTG, a nitric oxide [NO] donor and a reliable migraine trigger in migraineurs), and how this altered the distribution of various immune cells as well as Ccl2 and Ccr2 mRNA levels in mouse dura and TG, tissues that are strongly implicated in migraine pathophysiology, was examined. With Ccl2 and Ccr2 global knockout (KO) mice, it was investigated whether CCL2-CCR2 signaling is required for the development of chronic headache-related behavioral and cellular sensitization. This was followed by pharmacological blockade of CCL2-CCR2 signaling to assess the relative contribution of peripheral and central CCL2-CCR2 pathway in two mouse models of migraine. Using RNAscope in situ hybridization and immunohistochemistry in wild-type and various transgenic mouse lines, the main cell types that express CCL2 and/or CCR2 were identified in TG and dura. Next, cell type-specific deletion of Ccr2 was conducted to elucidate whether activation of CCR2 in T cells, macrophages and/or primary afferent neurons contributes to chronic headache-related sensitization. Lastly, whether CCL2-CCR2 signaling interacts with the CGRP pathway under normal and disease states, and whether inhibition of both CCL2 and CGRP signaling is more effective in reversing chronic headache-related behaviors than targeting individual pathways alone, was investigated.

Results

Repeated NTG Administration Increases Ccl2 and Ccr2 mRNA Expression in Mouse Dura and TG.

Migraine can be triggered by multiple mechanisms, for example, through enhanced NO signaling. Infusion of NTG, a NO donor, to migraine patients triggers a delayed headache and cutaneous hypersensitivity, both of which are attenuated by anti-migraine drugs. In rodents, repeated systemic administration of NTG mimics the induction of high frequency headache. This results in persistent cutaneous hypersensitivity that is blocked by migraine preventive therapy, suggesting that these behaviors are mechanistically related to chronic headache in humans. Repeated NTG increases the number of T cells in TG but not DRG, suggesting that neuroimmune interactions contribute to headache chronification. Herein was investigated whether repeated NTG administration alters the distribution of other immune cells in mouse dura and TG.

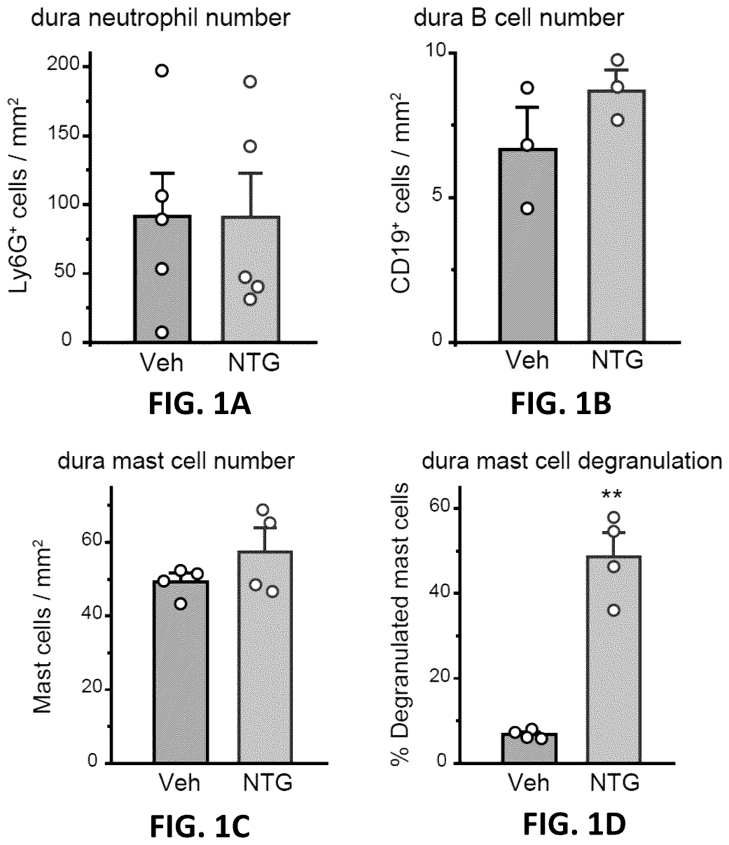

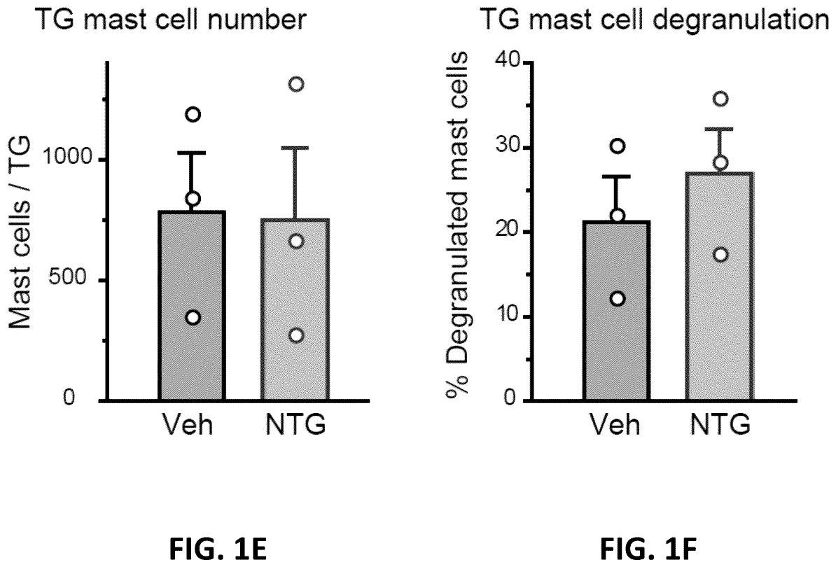

Female mice received i.p. injections of 10 mg/kg NTG or vehicle every two days for 5 times. Two days later, whole-mount dura as well as TG and lumbar DRG sections were stained with antibodies that recognize various immune cells. In dura surrounding the middle meningeal artery, the densities of Ly6G⁺ neutrophils, CD19⁺ B cells and mast cells were not altered by repeated NTG, but the percentage of degranulated mast cells was significantly increased as reported previously (see e.g., FIG. 1A-FIG. 1D). There were few neutrophils and B cells in TG from vehicle- or NTG-treated mice. Repeated NTG did not change the number or the degranulation of mast cells in TG (see e.g., FIG. 1E-FIG. 1F).

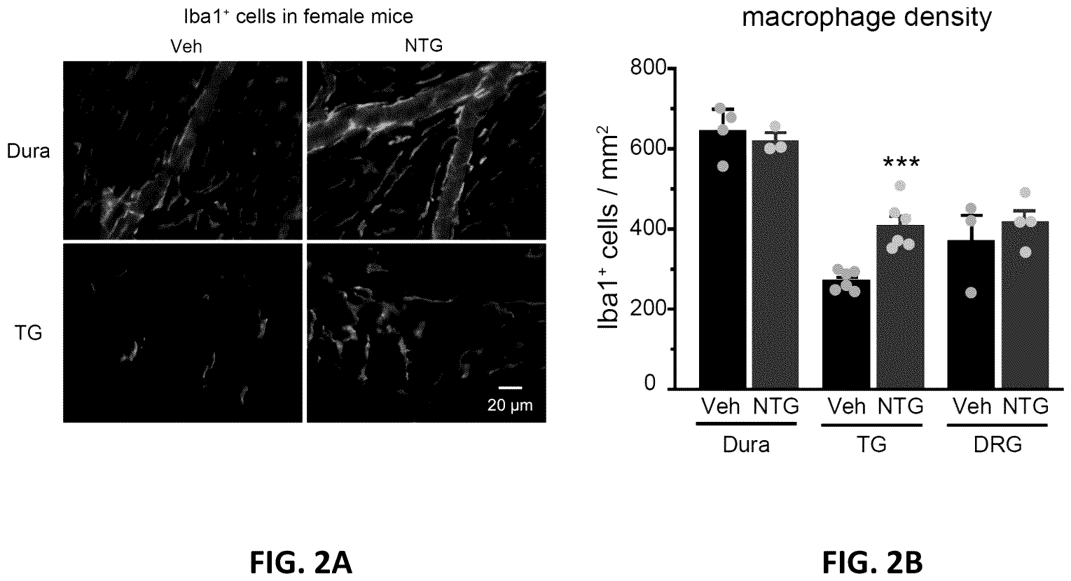

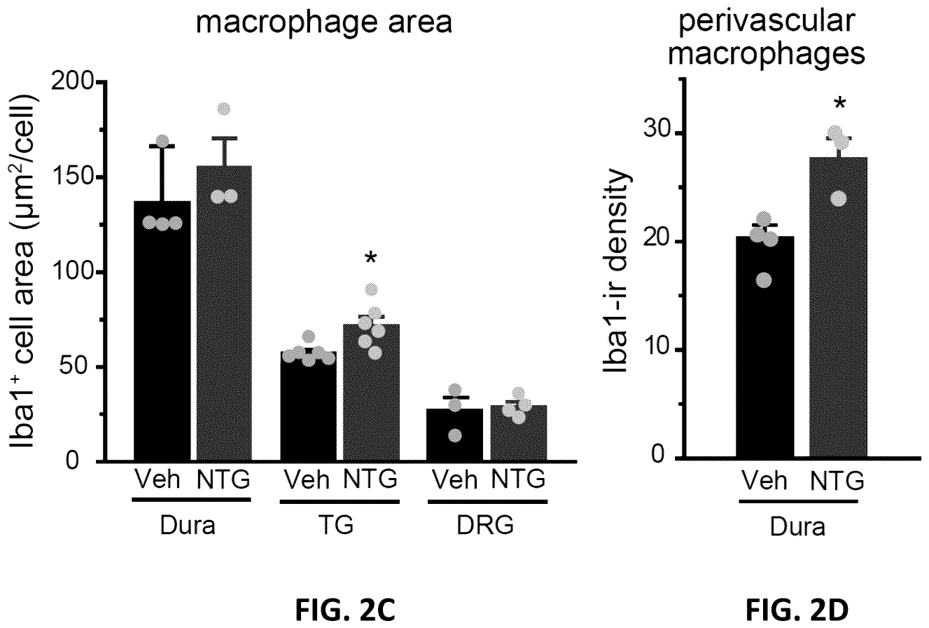

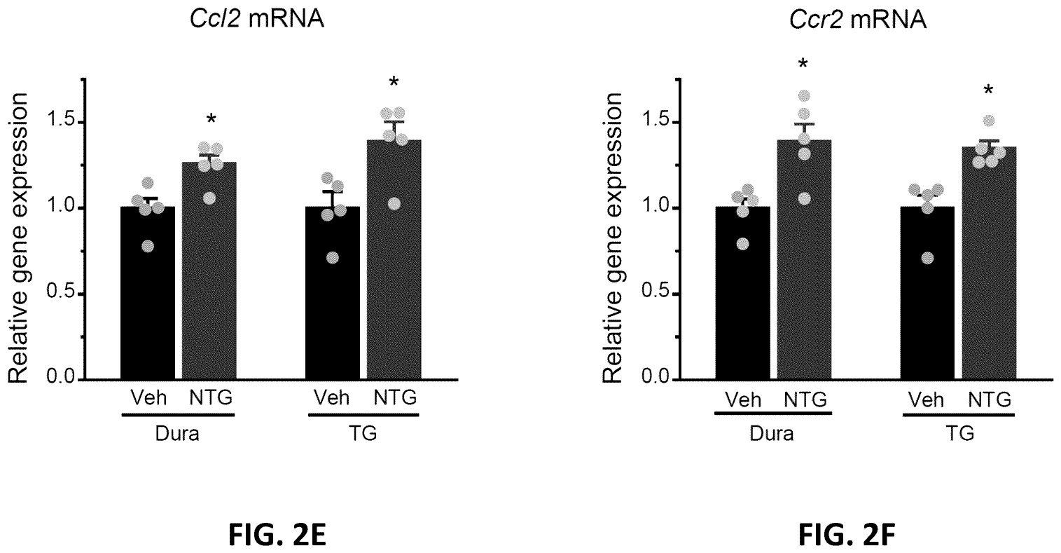

Macrophages were identified by the ionized calcium binding adaptor molecule 1-immunoreactivity (Iba1-ir, see e.g., FIG. 2A). Both the density and the area of Iba1⁺ cells were significantly increased in TG but not DRG after repeated NTG (see e.g., FIG. 2B-FIG. 2C). The perivascular and non-perivascular Iba1⁺ cells were quantified in dura separately, as they may serve distinct functions. Repeated NTG did not alter the density or the area of non-perivascular Iba1⁺ cells (see e.g., FIG. 2B-FIG. 2C), but significantly increased the density of perivascular Iba1-ir (see e.g., FIG. 2D). Since CCL2-CCR2 signaling is instrumental for the migration and infiltration of monocytes/macrophages to target tissues, the effects of repeated NTG on Ccl2 and Ccr2 mRNA expression in female mice were examined. The levels of Ccl2 and Ccr2 mRNA were significantly elevated in dura and TG from NTG-treated mice (see e.g., FIG. 2E-FIG. 2F), suggesting an enhanced CCL2-CCR2 signaling in these tissues during chronic headache.

Loss of Ccl2 or Ccr2 Abolishes NTG-Induced Behavioral Sensitization.

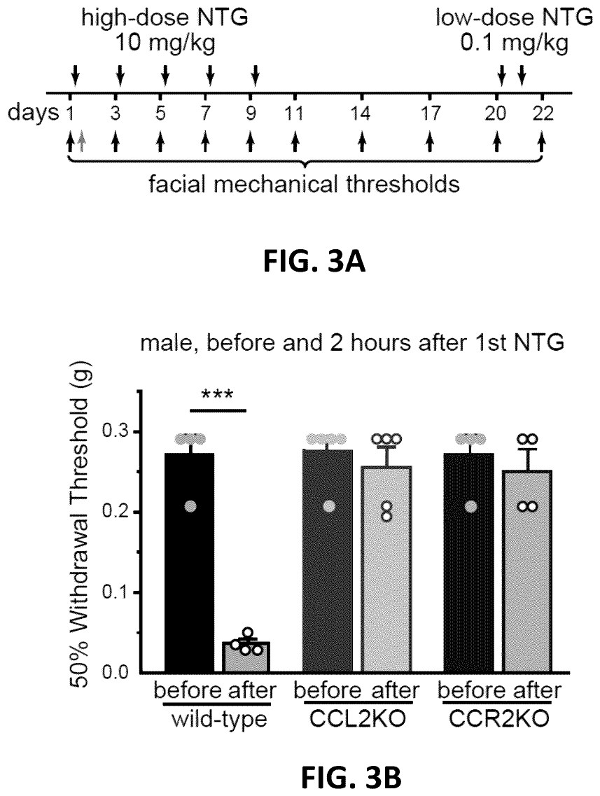

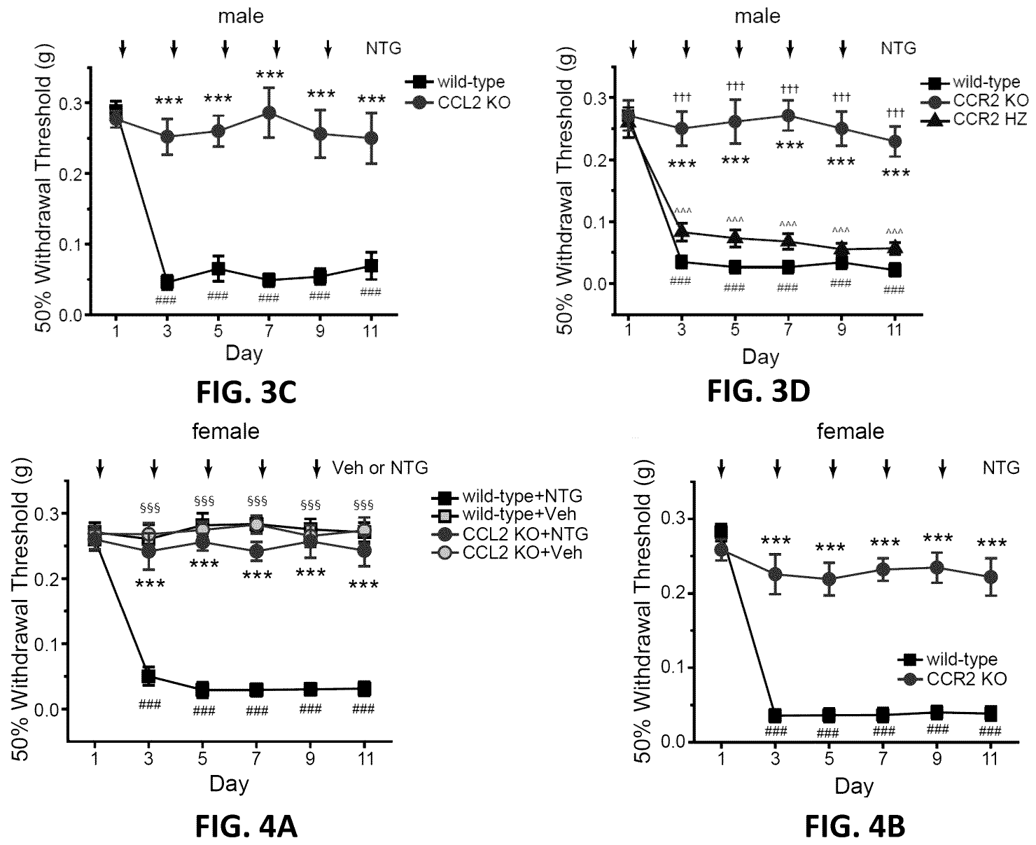

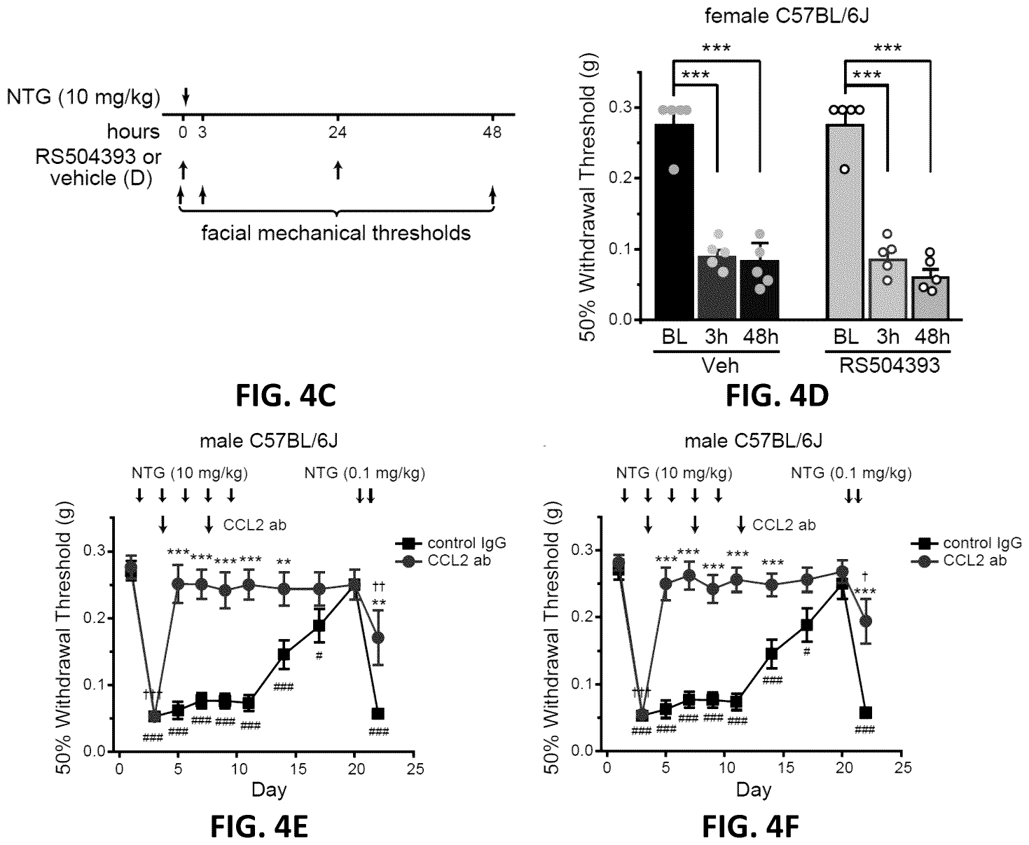

To investigate whether CCL2-CCR2 signaling contributes to chronic headache, the responses of wild-type (C57BL/6J), Ccl2 global KO, and Ccr2 global KO mice to single or repeated NTG administration were compared (10 mg/kg, i.p., see e.g., FIG. 3A). The baseline responses to mechanical stimuli on periorbital skin were comparable between wild-type and KO mice (see e.g., FIG. 3B-FIG. 3D), and were not altered by repeated vehicle injection (see e.g., FIG. 4A). In wild-type mice, facial mechanical thresholds were significantly reduced 2 hours after the first NTG injection (see e.g., FIG. 3B). The facial mechanical hypersensitivity persisted for 2 days and was sustained by the subsequent NTG injections in both male and female wild-type mice (see e.g., FIG. 3C-FIG. 3D). Conversely, Ccl2 and Ccr2 KO mice did not exhibit facial mechanical hypersensitivity after either single or repeated NTG administration regardless of sex (see e.g., FIG. 3B-FIG. 3D and FIG. 4A-FIG. 4B), indicating that CCL2-CCR2 signaling is required for the development of NTG-induced acute and persistent behavioral sensitizations in both male and female mice.

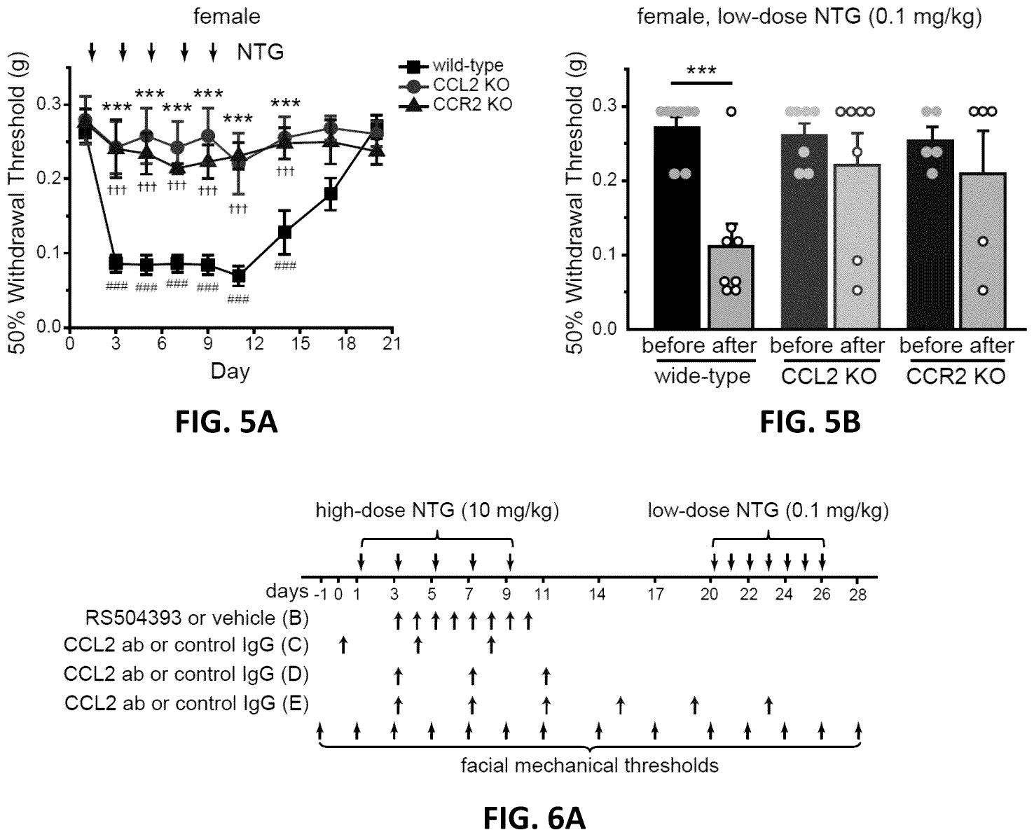

In wild-type mice, facial mechanical threshold returned to baseline level after the cessation of repeated NTG administration (see e.g., FIG. 5A, day 20). Subsequent injections of low-dose NTG (0.1 mg/kg, i.p.) re-established the facial mechanical hypersensitivity (see e.g., FIG. 5B), revealing the presence of hyperalgesic priming. Low-dose NTG did not alter facial mechanical thresholds in Ccl2 or Ccr2 KO mice that had received repeated high-dose NTG (see e.g., FIG. 5B), suggesting that CCL2-CCR2 signaling contributes to the development and/or the expression of hyperalgesic priming.

Peripheral CCL2-CCR2 Signaling Contributes to Repeated NTG-Induced Behavioral Sensitization.

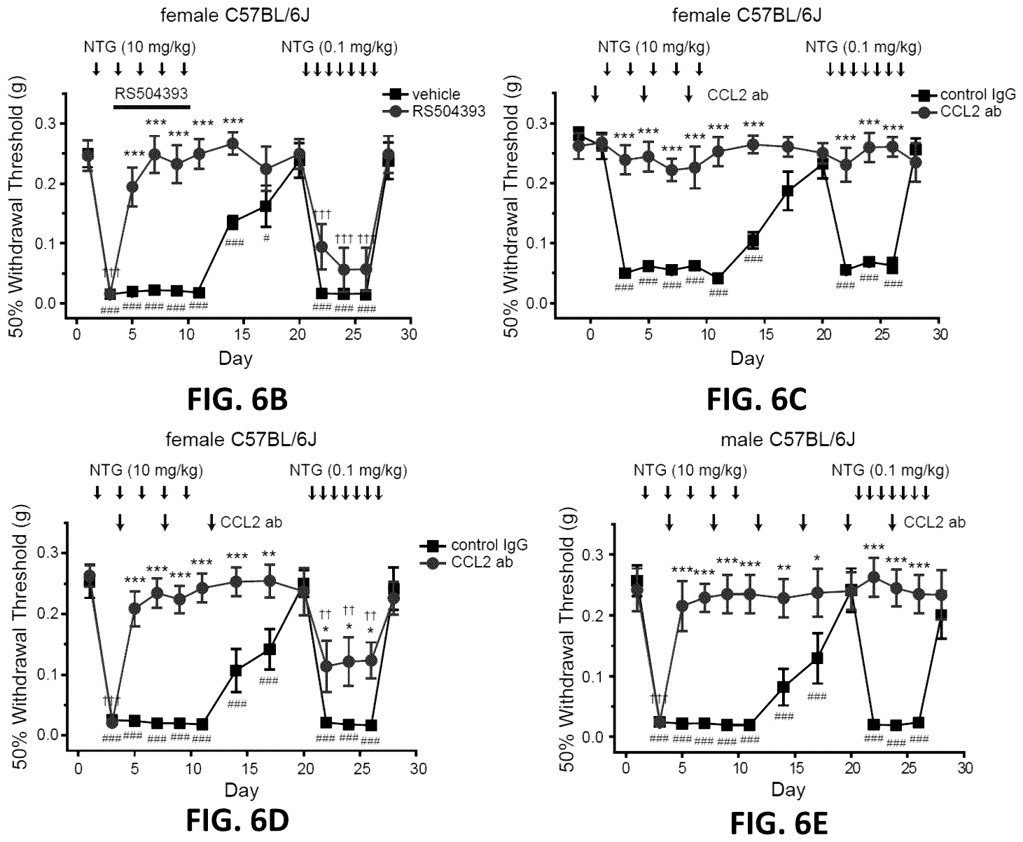

Global gene deletion may induce compensatory changes that confound the phenotypic analysis. To address this possibility, the CCL2-CCR2 signaling was inhibited pharmacologically. This also allowed for investigation of whether blocking CCL2-CCR2 signaling reverses chronic headache-related behavioral sensitization. Female C57BL/6J mice received 8 daily injections of the selective CCR2 antagonist RS504393 (3 mg/kg, i.p.) after they exhibited NTG-induced persistent facial skin hypersensitivity (see e.g., FIG. 6A). The facial mechanical threshold returned to basal level after 2 days of RS504393 treatment (see e.g., FIG. 6B, day 5), and was not reduced by subsequent NTG injections (see e.g., FIG. 6B, day 7-11). However, after the mechanical thresholds returned to baseline level, low-dose NTG injections still produced robust facial skin hypersensitivity (see e.g., FIG. 6B, day 20-28), indicating that blocking endogenous CCL2-CCR2 signaling can reverse the established facial skin hypersensitivity but does not prevent the development of hyperalgesic priming.

Repeated high-dose NTG administration may sensitize facial skin afferents in addition to meningeal afferents through CCL2-CCR2 signaling. To test this possibility, RS504393 (3 μg in 10 μl) was injected under the mouse periorbital skin, one at 30 minutes before NTG administration and the other at 24 hours post-NTG (see e.g., FIG. 4C). Although intrathecal administration of 3 μg RS504393 significantly attenuated tibial fracture/cast immobilization-induced mechanical allodynia in mice, periorbital RS504393 treatments did not reduce NTG-induced acute or persistent facial skin hypersensitivity (see e.g., FIG. 4D), indicating that the effects of systemic RS504393 on NTG-induced behaviors (see e.g., FIG. 6B) unlikely resulted from the inhibition of facial skin afferent sensitization.

To understand the contributions of central versus peripheral CCL2-CCR2 signaling to headache chronification, the peripheral CCL2-CCR2 pathway was targeted with a commercially available neutralizing antibody against mouse CCL2 (CCL2 ab, 200 μg/mouse [about 10 mg/kg] every 4 days, see e.g., FIG. 6A). First, neutralizing CCL2 before and during repeated NTG administration prevented the development of facial mechanical hypersensitivity and hyperalgesic priming in female mice (see e.g., FIG. 6C), indicating that the peripheral CCL2-CCR2 signaling pathway contributes to the development of chronic headache-related sensitization. Secondly, the same duration of CCL2 ab treatment, when started after the second NTG injection, completely reversed NTG-induced facial skin hypersensitivity (see e.g., FIG. 6D, day 5-11) but only partially attenuated hyperalgesic priming (see e.g., FIG. 6D, day 20-28). The delayed and short-duration CCL2 ab treatments had similar effects on NTG-induced behaviors in male C57BL/6J mice (see e.g., FIG. 4E and FIG. 4F). Prolonging the CCL2 ab treatment in male mice abolished low-dose NTG-induced sensitization (see e.g., FIG. 6E, day 20-28), indicating that the expression of hyperalgesic priming also requires CCL2-CCR2 signaling. Collectively, these results support a crucial role of the peripheral CCL2-CCR2 signaling pathway in the development of repeated NTG-induced behavioral sensitization.

Endogenous CCL2 Activates CCR2 in T Cells and Macrophages.

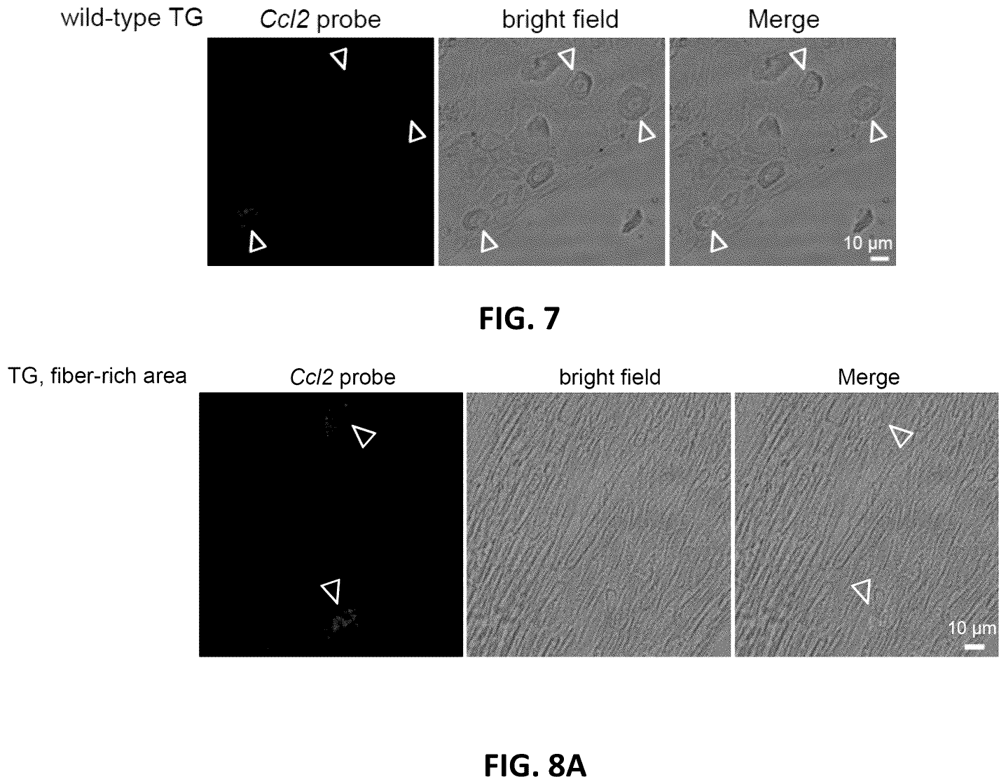

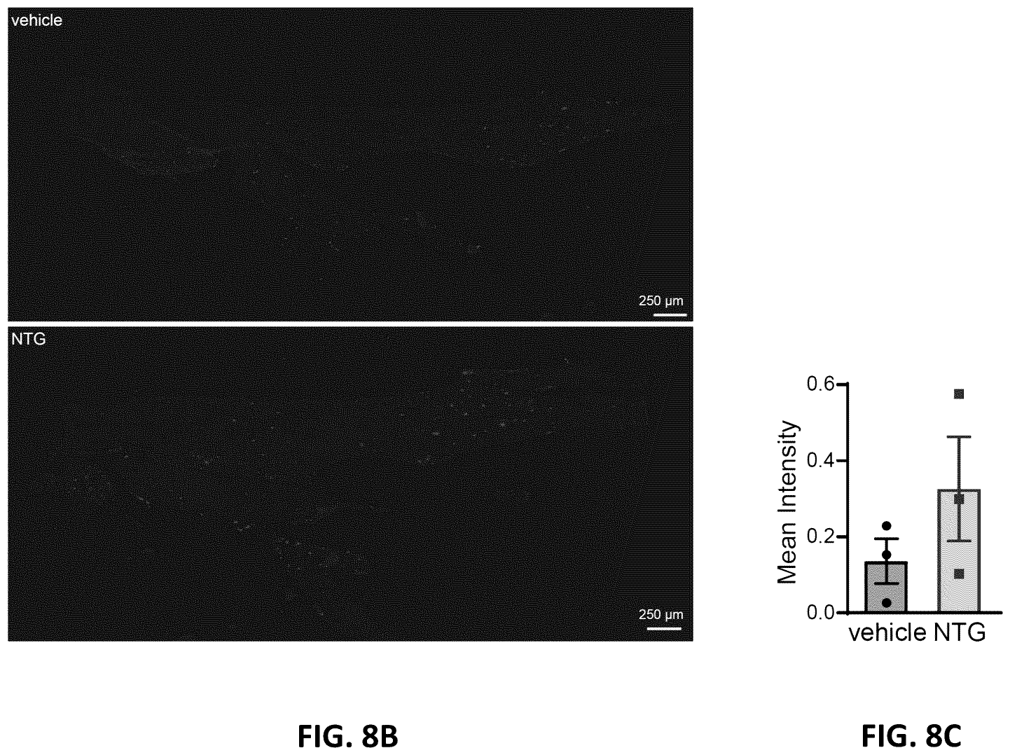

Many peripheral cells express CCL2 and/or CCR2. To identify the cellular source of CCL2 during chronic headache, RNAscope in situ hybridization was performed for mouse Ccl2 mRNA in TG sections. The majority of Ccl2 signal overlapped with the somas of TG neurons (see e.g., FIG. 7). The fiber-rich areas contained scattered Ccl2⁺ non-neuronal cells (see e.g., FIG. 8A). The mean Ccl2 signal intensity in neuron-rich areas was much higher than that in fiber-rich areas (9.0±5.3 fold, n=3 vehicle-treated mice). The level of Ccl2 mRNA in TG showed a trend of upregulation after repeated NTG, in line with the qPCR results (see e.g., FIG. 8B-FIG. 8C).

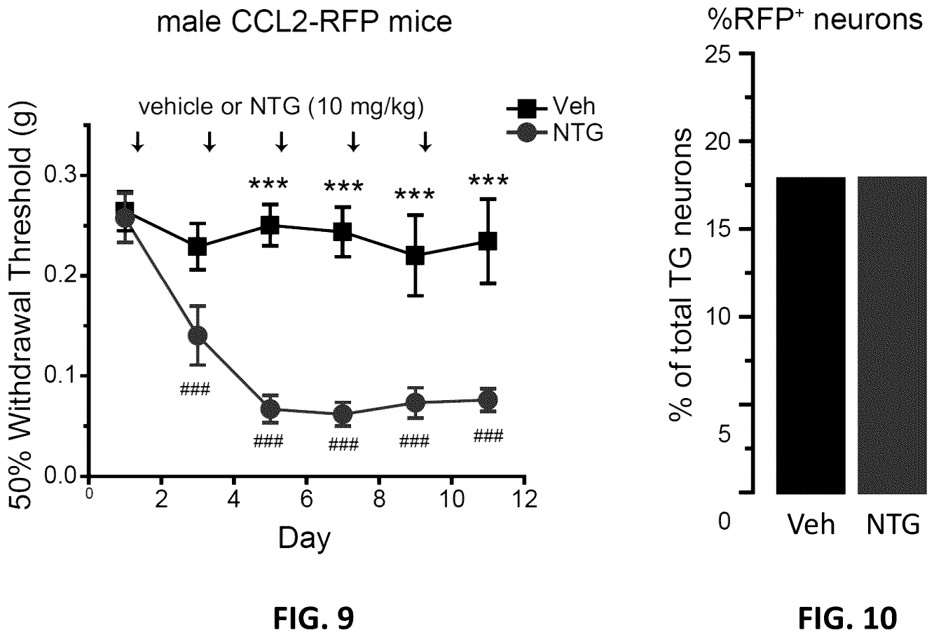

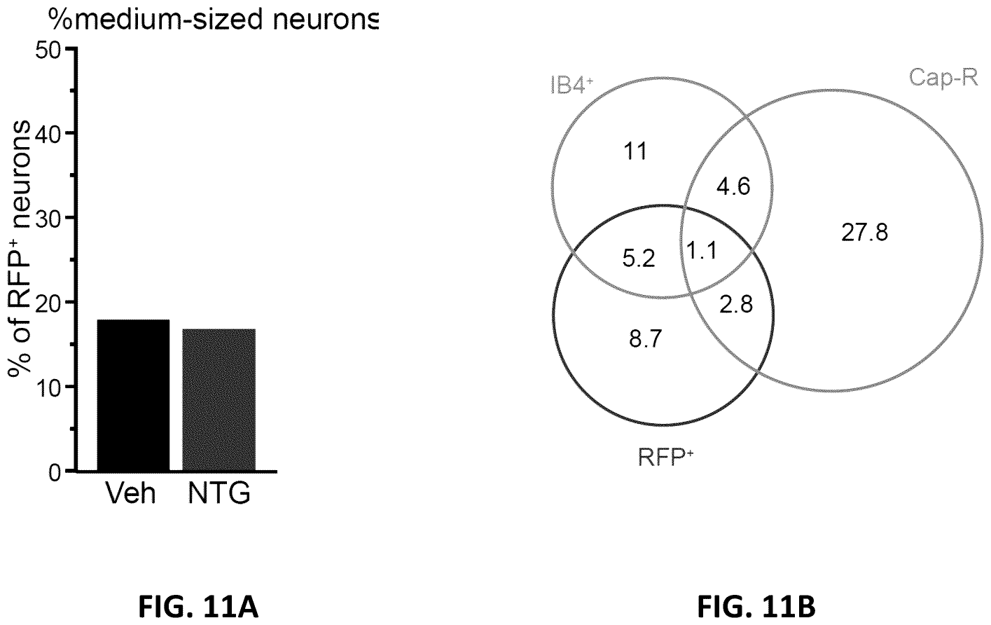

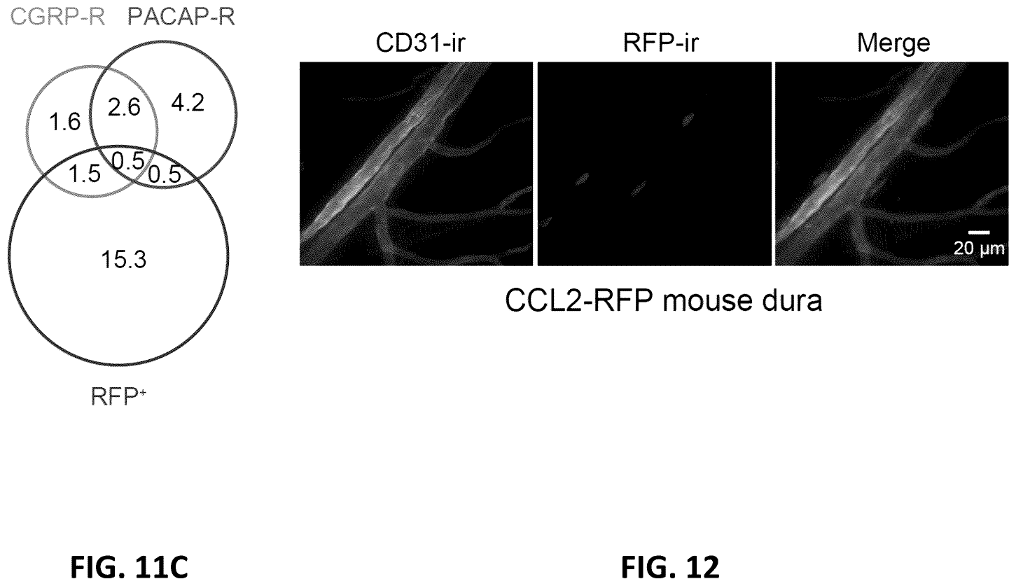

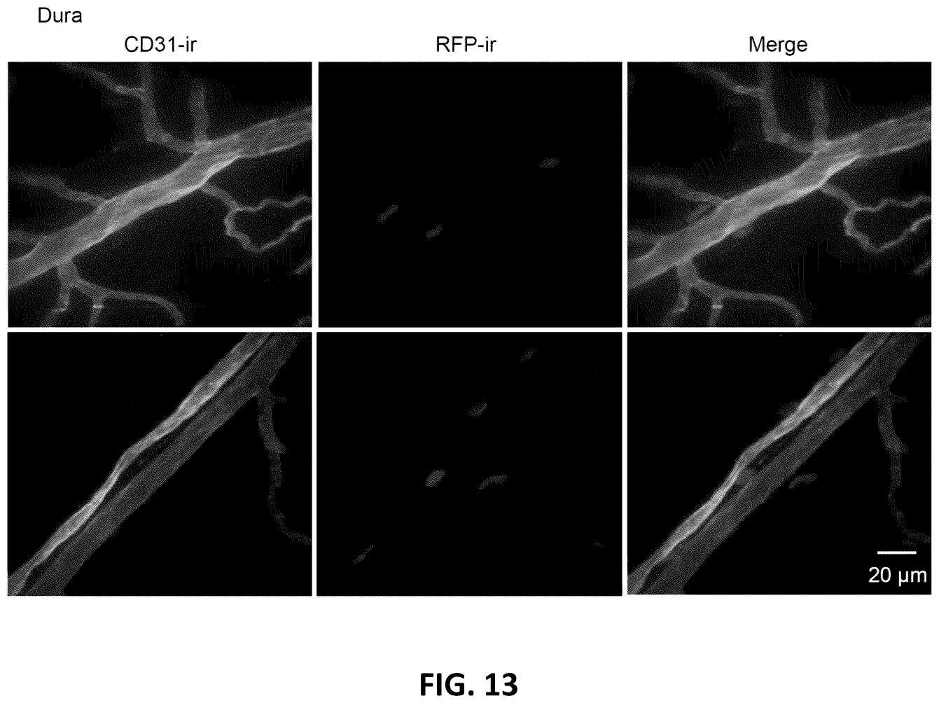

To further characterize CCL2-expressing TG neurons, cultured TG neurons from CCL2-RFP mice were used, which expressed both CCL2 and red fluorescent protein (RFP) from mouse Ccl2 alleles. Repeated NTG-induced sensitization was comparable between CCL2-RFP and C57BL/6J mice (see e.g., FIG. 9). About 18% of TG neurons were RFP⁺ in both vehicle- and NTG-treated groups (see e.g., FIG. 10), suggesting that repeated NTG does not alter the number of CCL2-expressing neurons but increases CCL2 expression in individual neurons and/or in non-neuronal cells. Among RFP⁺ TG neurons, more than 80% had small diameter somas (<25 μm), 35% bound to isolectin B4, and 22% responded to capsaicin (see e.g., FIG. 11A-FIG. 11C), suggesting that many CCL2-expressing TG neurons are nociceptors. Only 5-8% of RFP⁺ neurons exhibited Ca²⁺ transients in response to neuropeptides CGRP and pituitary adenylate cyclase-activating polypeptide (PACAP, see e.g., FIG. 11C), suggesting that the production and release of CCL2 from TG neurons are not regulated by CGRP or PACAP signaling. In whole-mount dura from CCL2-RFP mice, strong RFP signals were present in scattered cells closely associated with the blood vessels but did not overlap with the endothelial cell marker CD31 (see e.g., FIG. 12 and FIG. 13), reminiscent of previously reported CCL2-expressing meningeal mural cells located along intervals within the vascular basement membrane. RFP+ fibers were not observed in the dura, likely due to the low level of RFP in the axons of TG neurons.

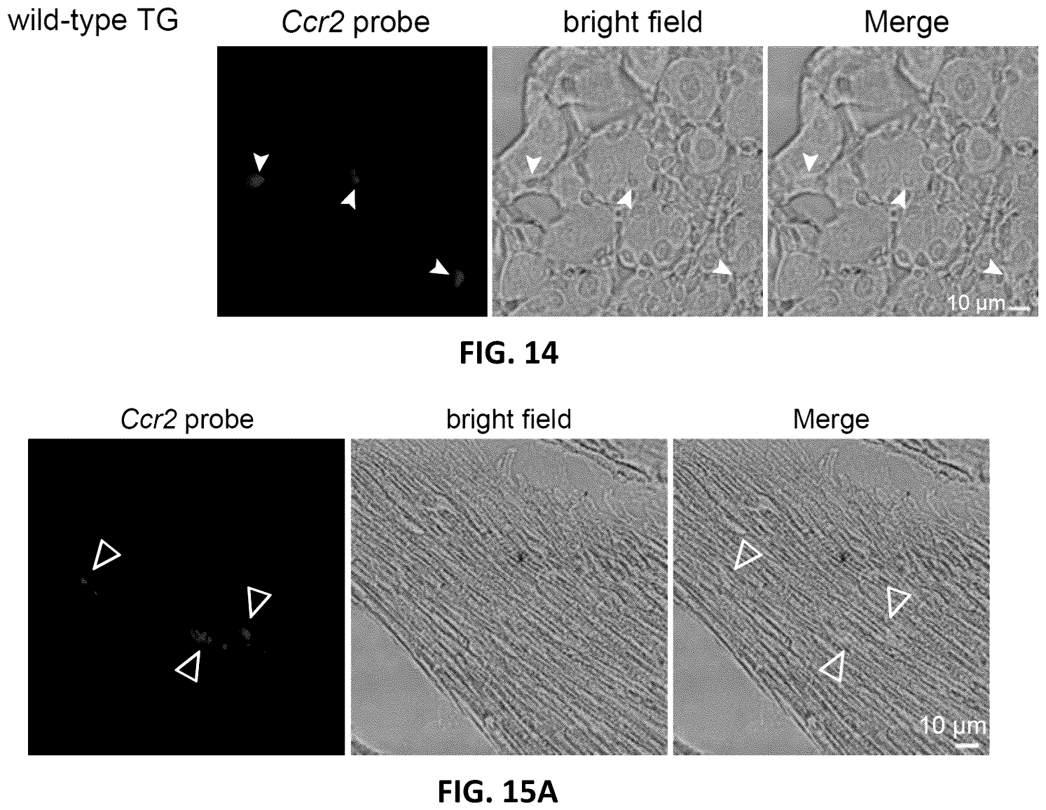

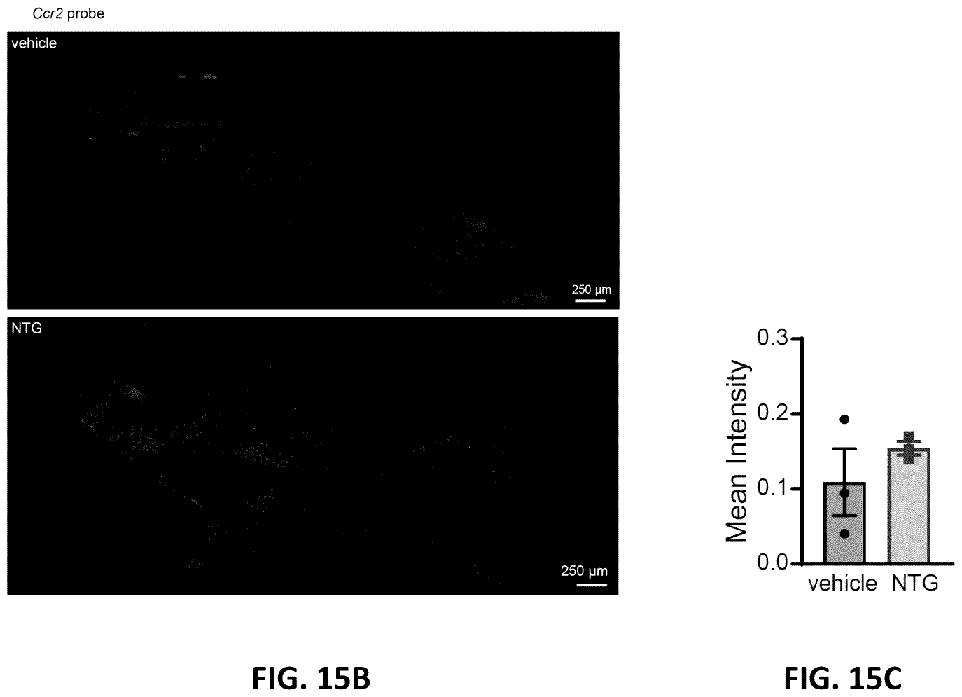

RNAscope in situ hybridization was also conducted for mouse Ccr2 mRNA in TG sections. Ccr2 signals were present in many non-neuronal cells (see e.g., FIG. 14 and FIG. 15A). The level of Ccr2 mRNA in TG showed a trend of upregulation after repeated NTG (see e.g., FIG. 15B-FIG. 15C). Surprisingly, few, if any, TG neurons from vehicle- or NTG-treated mice exhibited Ccr2 signal (see e.g., FIG. 14).

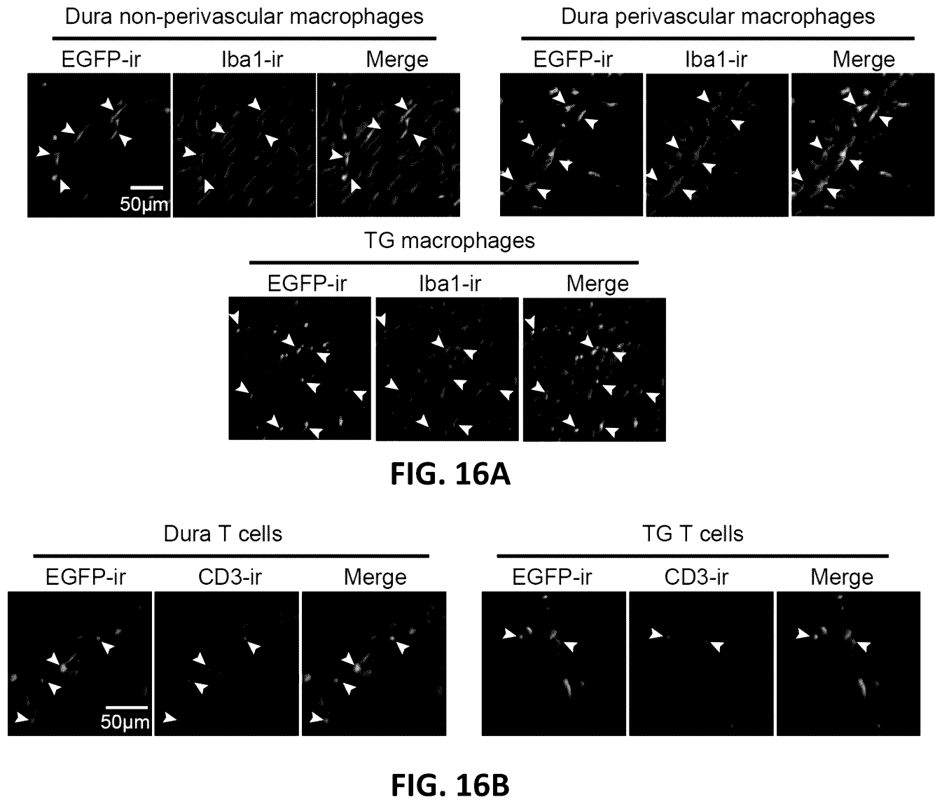

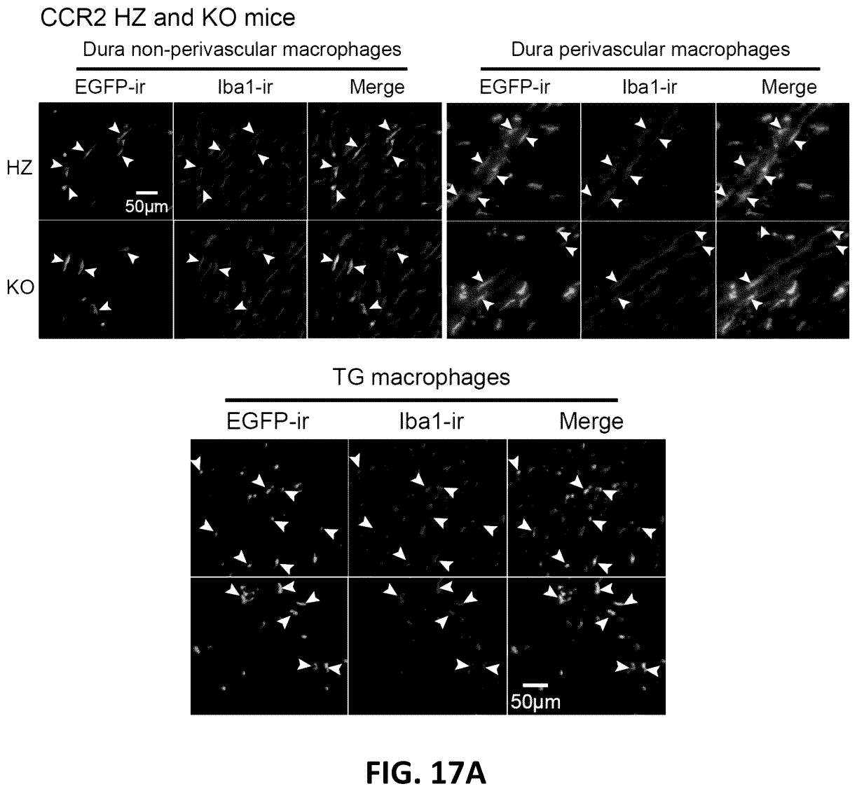

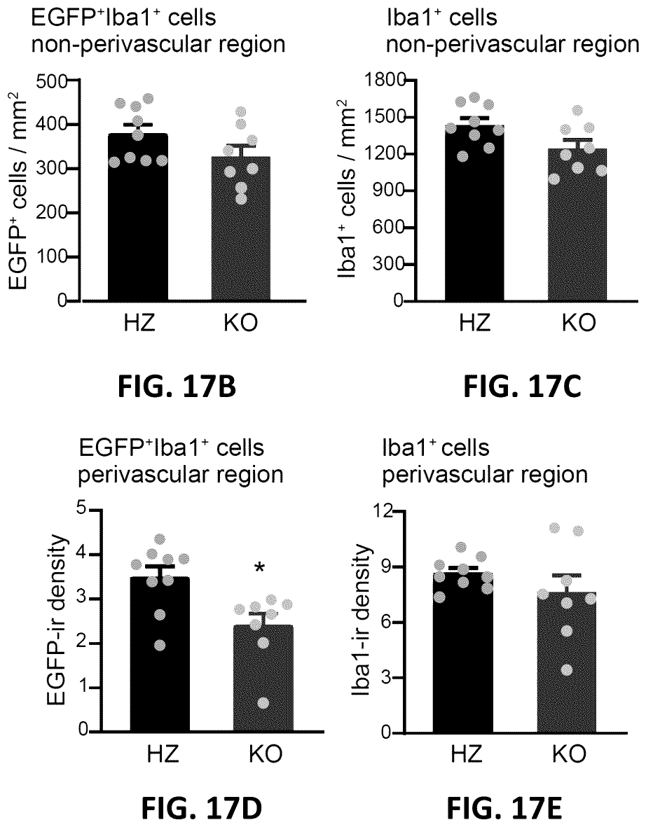

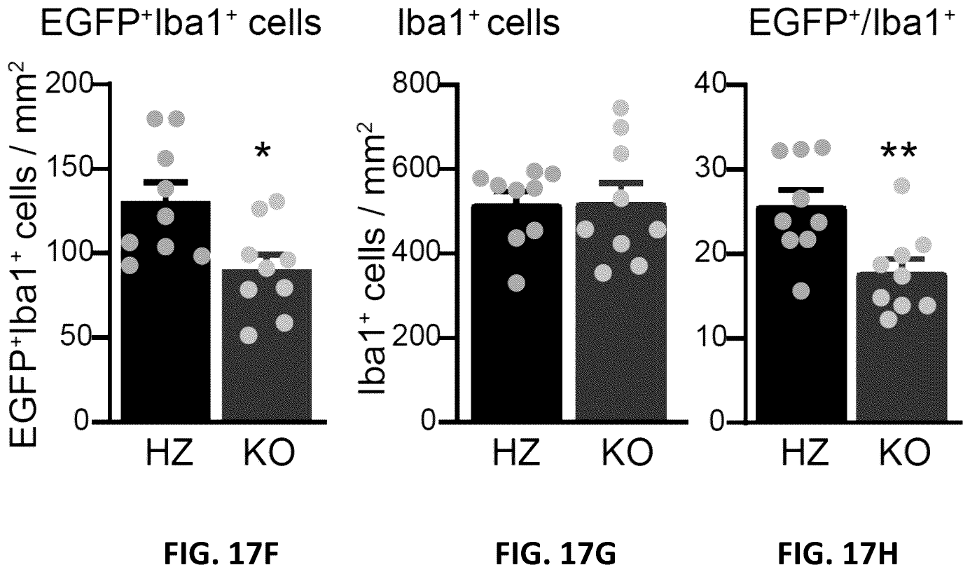

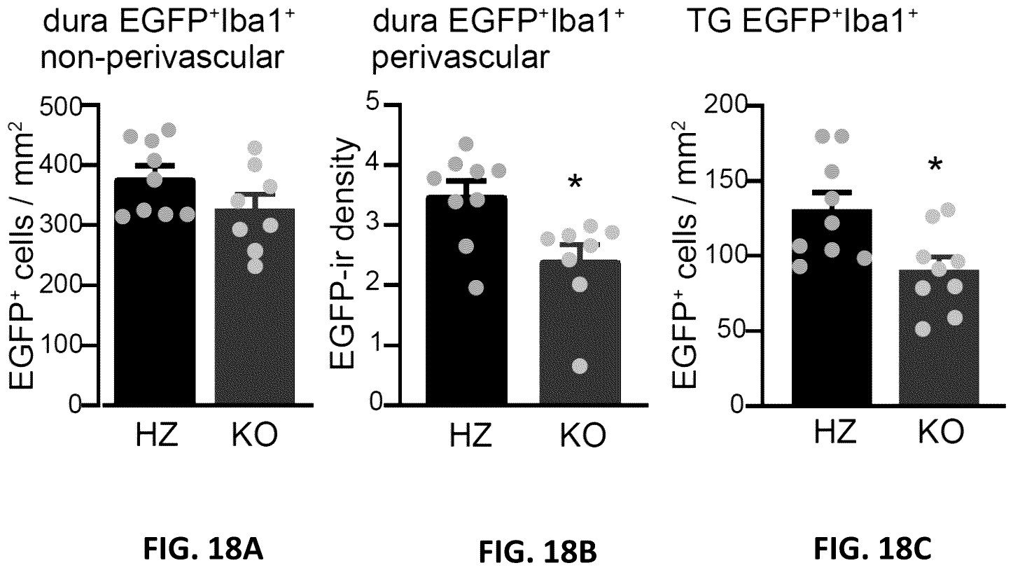

In many tissues, CCR2 is highly expressed in subsets of monocytes/macrophages and T cells. To further characterize CCR2-expressing cells in TG and dura, Ccr2 heterozygous (HZ) and KO mice were used, which express EGFP from one or both mouse Ccr2 alleles. In TG and dura tissues from Ccr2 HZ mice, strong EGFP-immunoreactivity (EGFP-ir) was present in ˜25% of Iba1⁺ macrophages and T cells identified by CD3-immunoreactivity (CD3-ir, see e.g., FIG. 16A-FIG. 16B and FIG. 17A-FIG. 17H). Although the density of total Iba1⁺ macrophages were comparable between Ccr2 HZ and KO groups (see e.g., FIG. 17C, FIG. 17E, and FIG. 17G), KO tissues had much lower density of EGFP⁺Iba1⁺ cells in TG and in the perivascular regions of dura (see e.g., FIG. 18A-FIG. 18C, FIG. 17B, FIG. 17D, FIG. 17F, and FIG. 17H). These results suggest that CCL2-CCR2 signaling contributes to the establishment of a subset of resident macrophages in dura and TG.

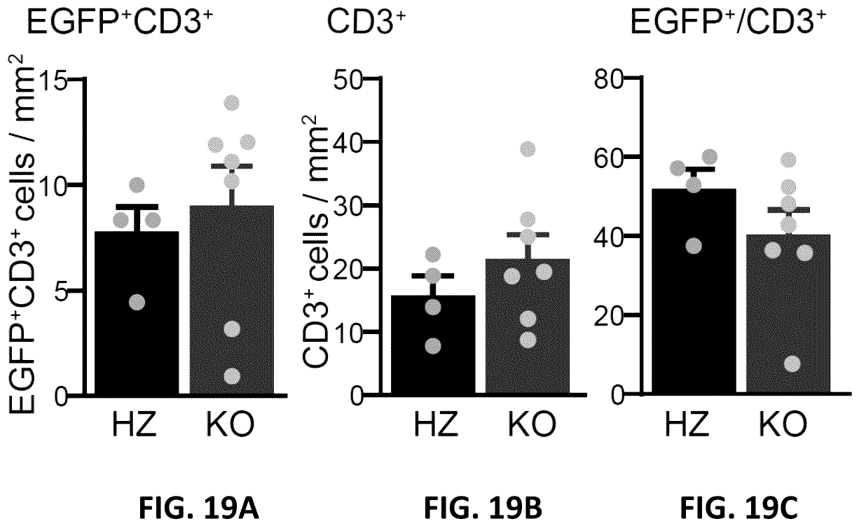

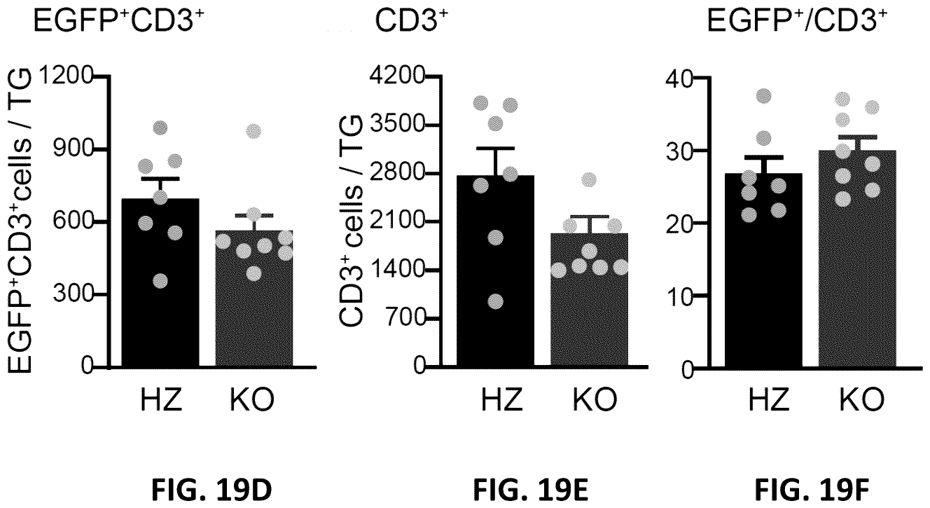

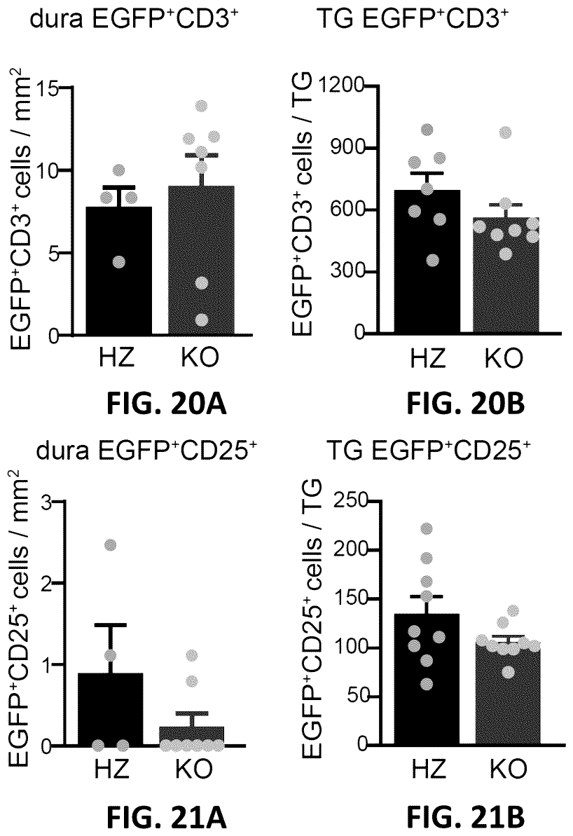

The dura and TG from Ccr2 HZ mice contained about 50% and 27% of EGFP⁺ cells among CD3⁺ T cells (see e.g., FIG. 19A-FIG. 19F), much higher than that in the blood, spleen, or lymph nodes (1-15%). The numbers of EGFP⁺CD3⁺ and CD3⁺ cells were comparable between Ccr2 HZ and KO tissues (see e.g., FIG. 20A-FIG. 20B), suggesting that CCR2 is not the predominant receptor mediating T cell homing to dura and TG. This is consistent with the previous report that loss of Ccr2 does not impair T cell entry into the colon in a mouse model of colitis.

Loss of Ccr2 also increases immunosuppressive regulatory T (Treg) cells in inflamed colon. CD25-immunoreactivity was used to identify Treg cells and found that all CD25⁺ cells in Ccr2 HZ tissues exhibited EGFP-ir, indicating that all Treg cells in dura and TG express CCR2. Ccr2 HZ and KO tissues contained similar numbers of EGFP⁺CD25⁺ cells (see e.g., FIG. 21A-FIG. 21B), indicating that loss of Ccr2 does not lead to Treg cell expansion in dura or TG.

Collectively, these data suggest that repeated NTG administration releases CCL2 from TG neurons and meningeal mural cells. This, in turn, activates CCR2 in T cells and macrophages in TG and dura.

Development of NTG-Induced Behavioral Sensitization Requires CCR2 Signaling in Both T Cells and Macrophages.

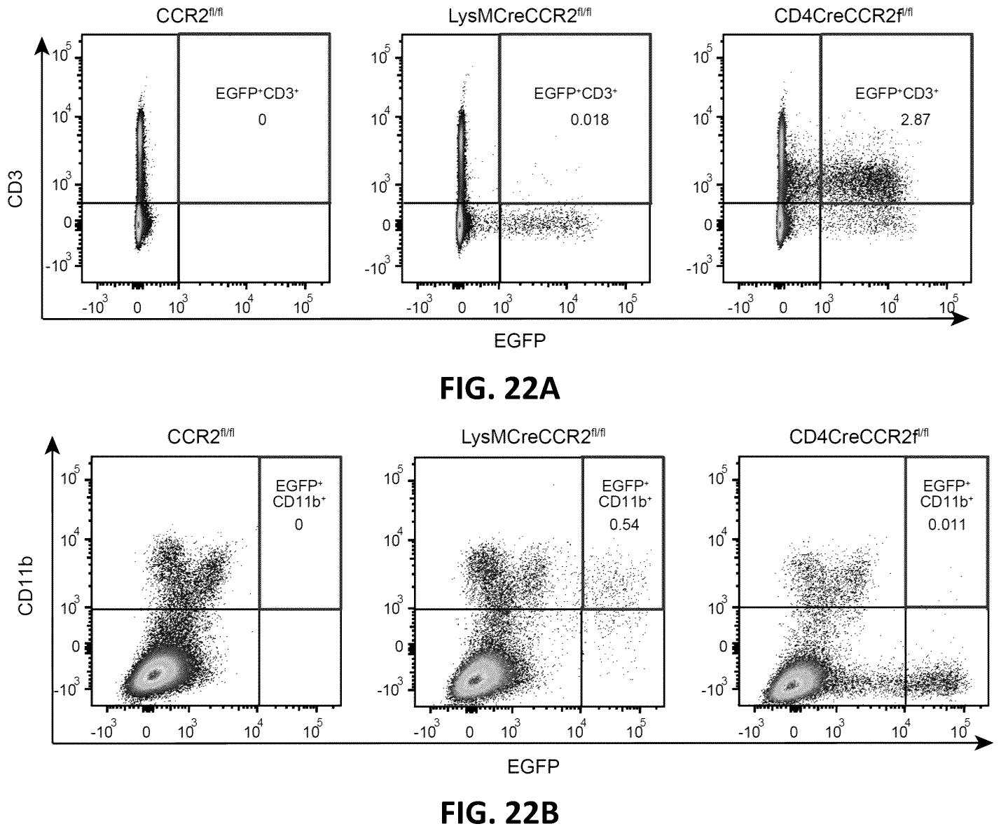

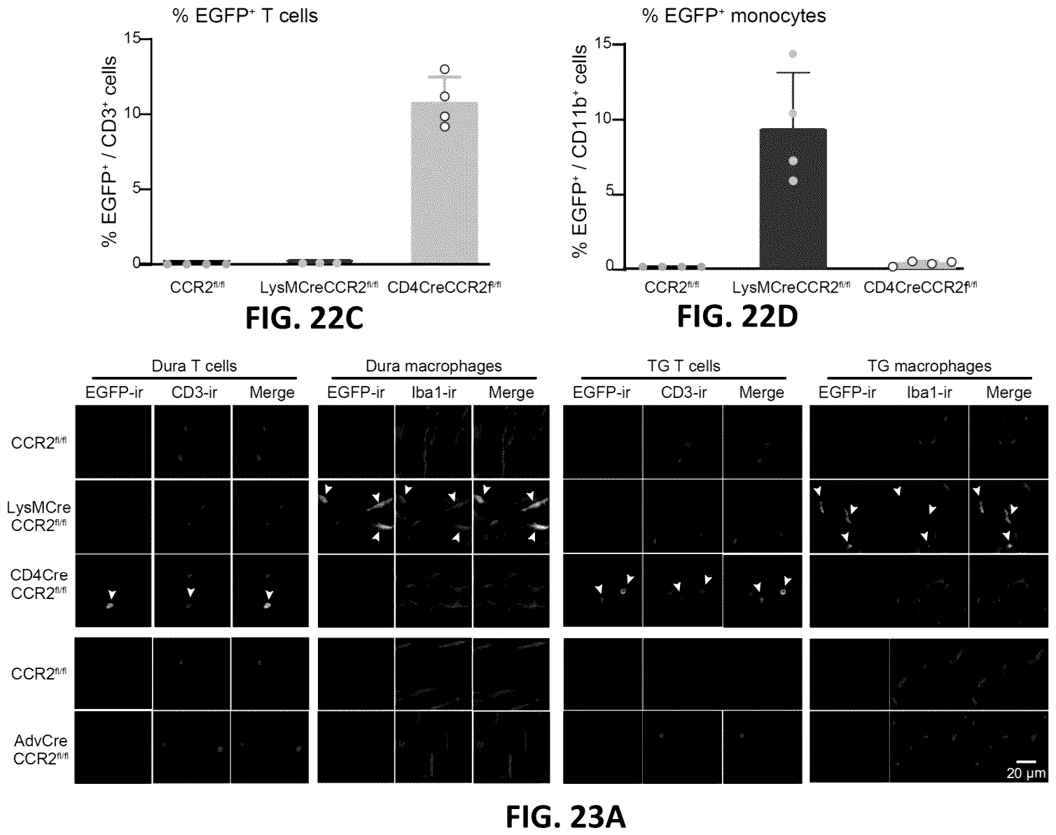

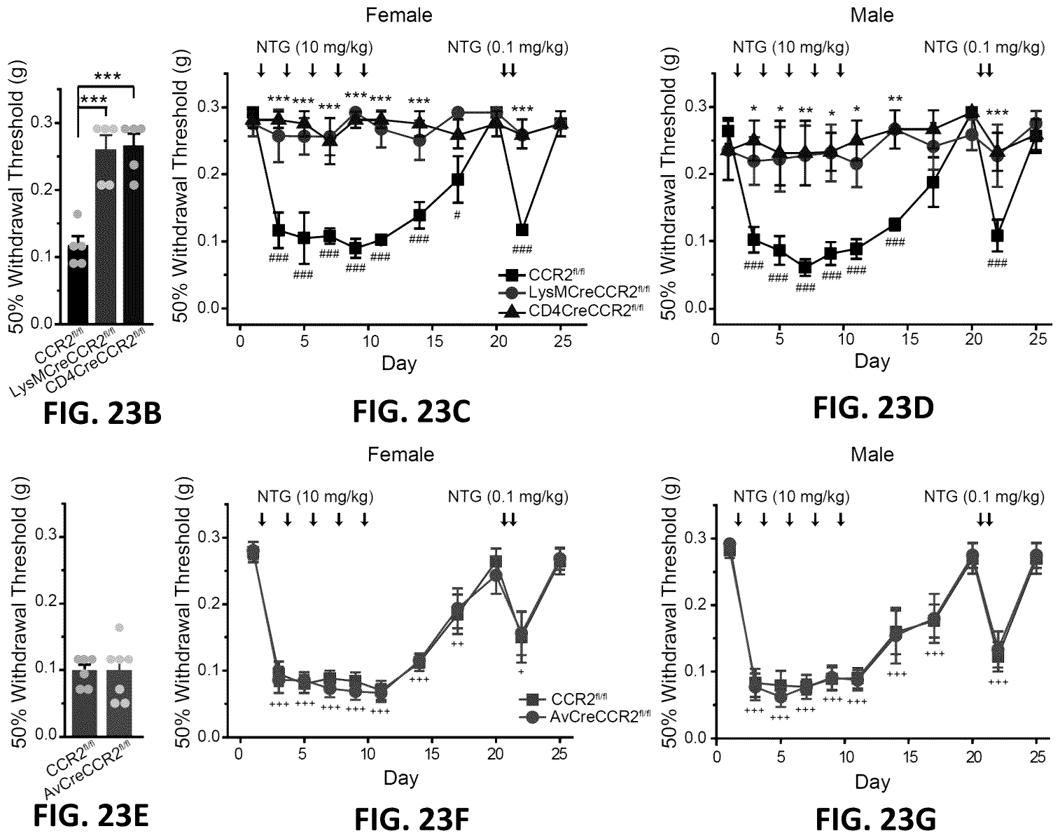

To understand the role of CCR2 signaling pathway in T cells and/or macrophages in headache chronification, CD4CreCcr2^{fl/fl} and LysMCreCcr2^{fl/fl} mice were generated to selectively eliminate CCR2 expression in T cells and myeloid cells, respectively. In addition to abolishing CCR2 expression, Cre-mediated recombination enabled EGFP expression in Ccr2-deficient cells. Indeed, the peripheral blood from CD4CreCcr2^{fl/fl} mice contained significant amount of EGFP⁺CD3⁺ cells but few EGFP⁺CD11b⁺ monocytes (see e.g., FIG. 22A-FIG. 22D). In dura and TG tissues from CD4CreCcr2^{fl/fl} mice, EGFP-ir overlapped with CD3-ir but not Iba1-ir (see e.g., FIG. 23A), indicating a selective deletion of Ccr2 in T cells (see e.g., FIG. 22A-FIG. 22D). In blood cells from LysMCreCcr2^{fl/fl} mice, ˜10% of CD11b⁺ cells were EGFP⁺ but few CD3⁺ cells were EGFP+(see e.g., FIG. 22A-FIG. 22D). EGFP-ir in dura and TG tissues from LysMCreCcr2^{fl/fl} mice overlapped with Iba1-ir but not CD3-ir (see e.g., FIG. 23A), indicating a selective Ccr2 deletion in monocytes/macrophages versus T cells.

The responses of Ccr2^{fl/fl}, CD4CreCcr2^{fl/fl} and LysMCreCcr2^{fl/fl} mice were compared to single or repeated administration of NTG (10 mg/kg, i.p.). Similar to C57BL/6J mice, the facial mechanical threshold in Ccr2^{fl/fl} mice was significantly reduced 2 hours after the first NTG injection (see e.g., FIG. 23B). Repeated NTG administration resulted in persistent facial mechanical hypersensitivity and hyperalgesic priming in both female and male Ccr2^{fl/fl} mice (see e.g., FIG. 23C-FIG. 23D). Conversely, neither CD4CreCcr2^{fl/fl} nor LysMCreCcr2^{fl/fl} mice exhibited NTG-induced acute sensitization (see e.g., FIG. 23B), and repeated NTG failed to induce facial skin hypersensitivity or hyperalgesic priming in CD4CreCcr2^{fl/fl} or LysMCreCcr2^{fl/fl} mice regardless of sex (see e.g., FIG. 23C-FIG. 23D). These results indicate that CCR2 signaling in both T cells and macrophages are required to establish NTG-induced acute and persistent behavioral sensitization in male and female mice.

Although CCR2 expression was not observed in TG neurons, it is possible that CCR2 is expressed at very low level or is upregulated by repeated NTG at time points there were not examined. Therefore, AvCreCcr2^{fl/fl} mice were generated to preferentially delete Ccr2 in primary afferent neurons. There was little EGFP-ir in CD3⁺ or Iba1⁺ cells in dura or TG tissues from AvCreCcr2^{fl/fl} mice (see e.g., FIG. 23A), indicating that CCR2 expression in T cells and macrophages was not affected. EGFP-ir was absent in TG neurons from AvCreCcr2^{fl/fl} mice (see e.g., FIG. 23A), confirming no or extremely low CCR2 expression under control condition. The responses of AvCreCcr2^{fl/fl} mice to single or repeated NTG administration were indistinguishable from those of Ccr2^{fl/fl} mice (see e.g., FIG. 23E-FIG. 23G), supporting that the activation of CCR2 in T cells and macrophages but not in TG neurons contributes to NTG-induced behavioral sensitization.

Peripheral CCL2-CCR2 Signaling Mediates Repetitive Stress-Induced Behavioral Sensitization.

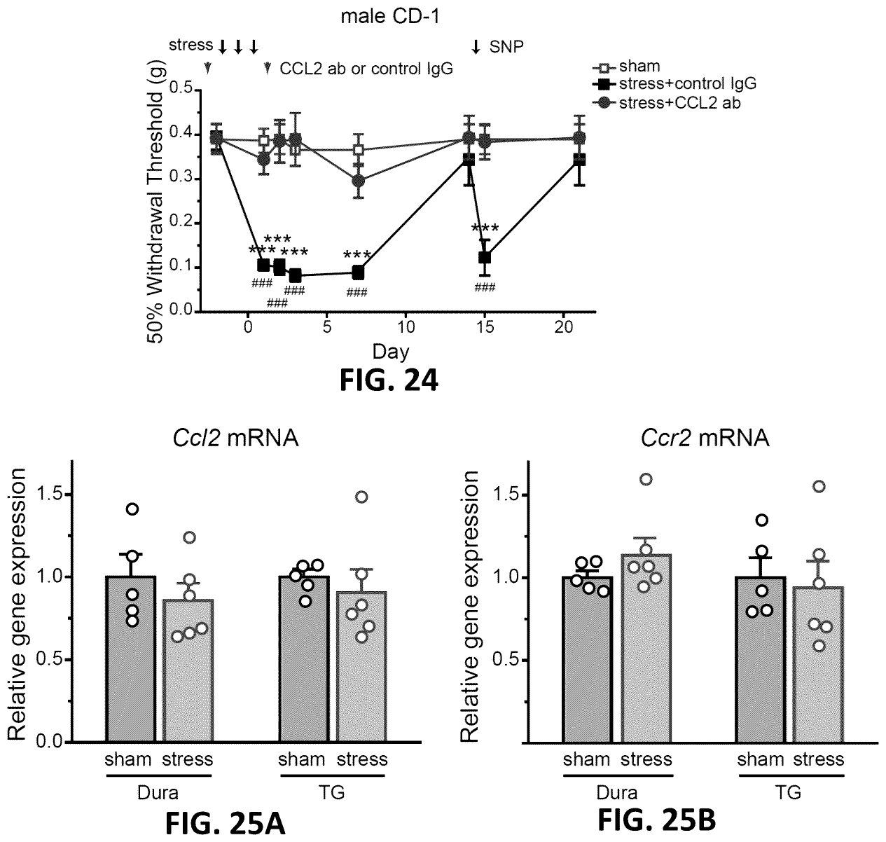

Repeated NTG administration models NO signaling-induced headache chronification. Stress, or its relief, is another major risk factor for headache chronification. To understand whether repetitive stress or its resolution engages the endogenous CCL2-CCR2 signaling pathway, male CD-1 mice were pretreated with the commercially available neutralizing CCL2 ab (200 μg/mouse every 4 days, i.p.) and then subjected them to 2 hours of restraint stress every morning for 3 consecutive days. In control IgG-treated mice, repetitive stress resulted in facial mechanical hypersensitivity that lasted for 2 weeks (see e.g., FIG. 24, stress+control IgG, day 1-14). After the mechanical thresholds returned to baseline level, i.p. injection of another NO donor SNP (0.3 mg/kg) revealed hyperalgesic priming in restrained mice but not in sham control (see e.g., FIG. 24, day 14-21) as in previous studies. CCL2 ab pre-treated mice did not develop stress-induced facial skin hypersensitivity or hyperalgesic priming (see e.g., FIG. 24, stress+CCL2 ab), suggesting a pivotal role of the peripheral CCL2-CCR2 signaling in establishing stress-induced behavioral sensitization. Interestingly, neither Ccl2 nor Ccr2 mRNA expression was altered in TG or dura 1 day after repetitive stress (see e.g., FIG. 25), suggesting that baseline CCL2-CCR2 signaling may be sufficient to mediate stress-induced sensitization.

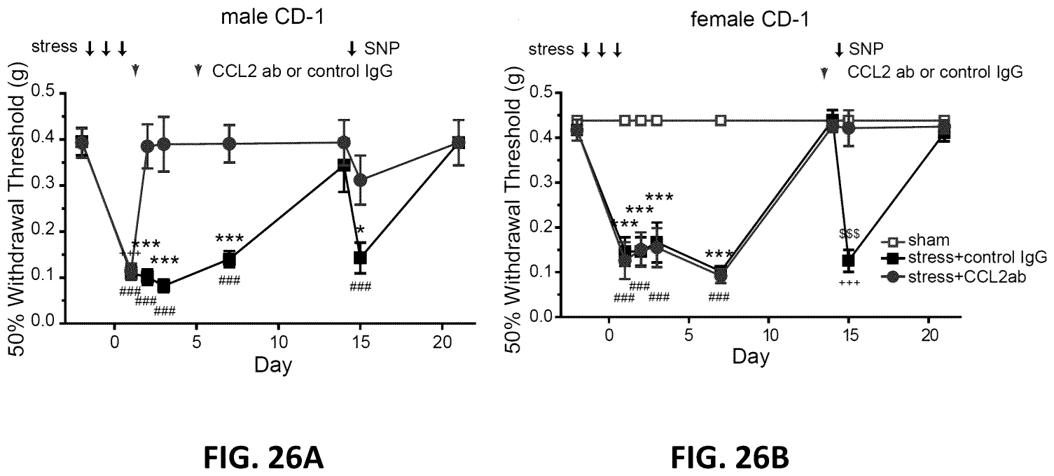

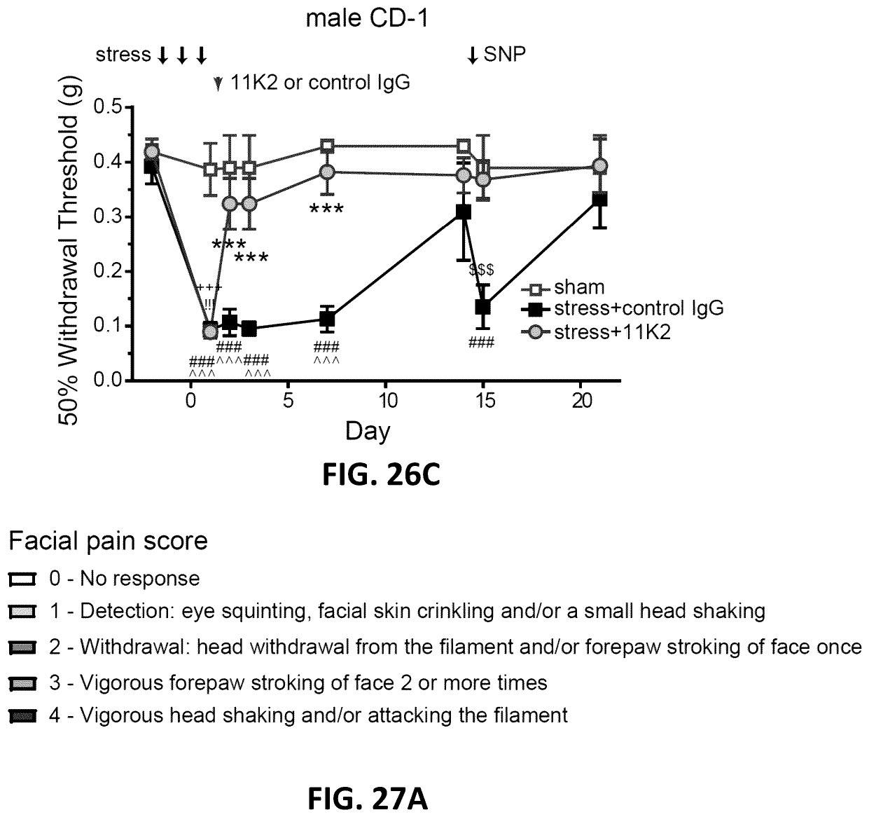

Next, repetitive stress-induced behavioral sensitization was established in male CD-1 mice, and then CCL2 ab treatment was initiated. This significantly accelerated the resolution of facial skin hypersensitivity (see e.g., FIG. 26A, day 1-14) and prevented hyperalgesic priming (see e.g., FIG. 26A, day 14-21), suggesting that peripheral CCL2-CCR2 signaling contributes to the maintenance of stress-induced behaviors. Lastly, female CD-1 mice were treated with CCL2 ab after the recovery of stress-induced acute sensitization (see e.g., FIG. 26B, day 1-14). This prevented SNP from re-establishing facial skin hypersensitivity (see e.g., FIG. 26B, day 14-21), suggesting that the expression of repetitive stress-induced hyperalgesic priming also requires peripheral CCL2-CCR2 signaling.

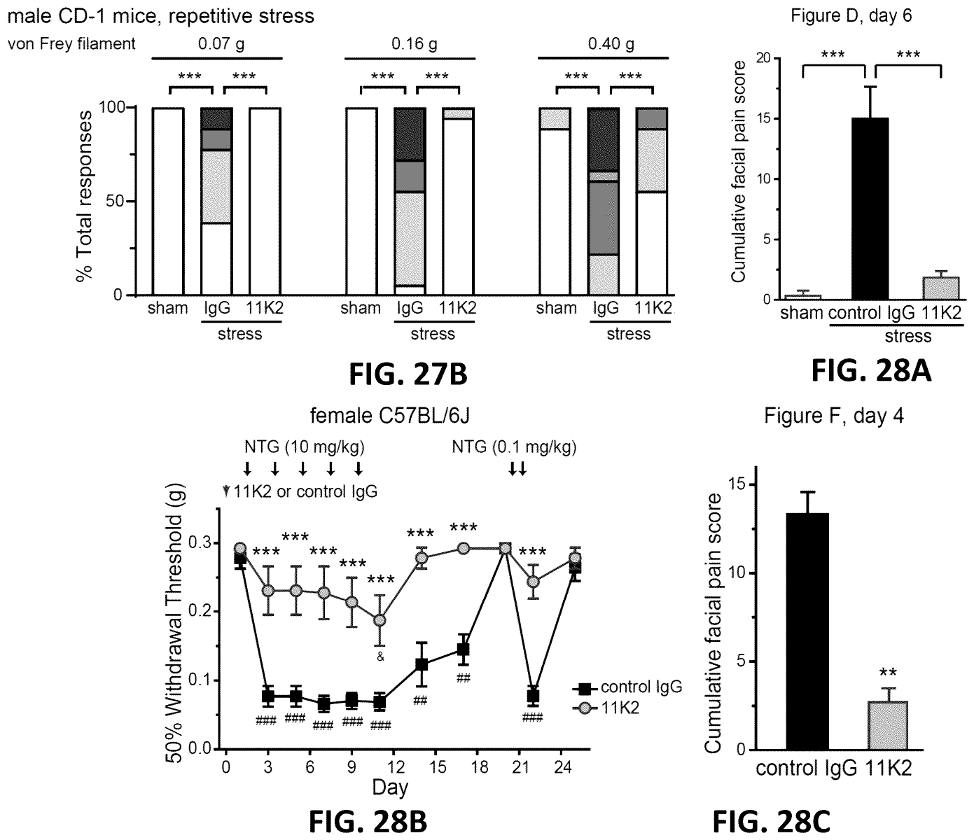

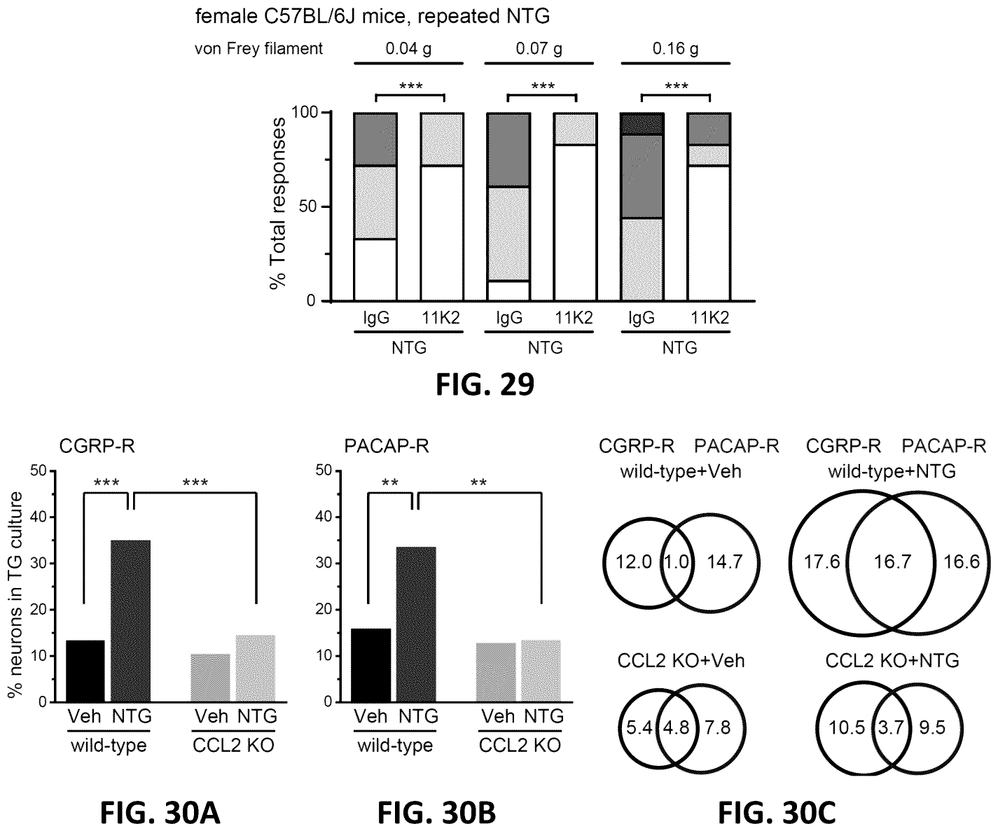

Collectively, peripheral CCL2-CCR2 signaling was shown to mediate repetitive stress-induced behavioral sensitization, and neutralizing peripheral CCL2 may alleviate stress-induced headache and prevent its chronification. To verify peripheral CCL2 as a potential target for headache treatment, another chimeric CCL2 neutralizing antibody was tested, 11K2, which binds to human CCL2 with very high affinity and also cross-reacts with mouse CCL2. A single 11K2 treatment (30 mg/kg, i.p.) was sufficient to completely reverse stress-induced facial skin hypersensitivity and prevent hyperalgesic priming (see e.g., FIG. 27A-FIG. 27B). Moreover, 11K2 inhibited stress-induced increase in facial pain score, which reflects higher aversiveness to mechanical stimuli on facial skin (see e.g., FIG. 28A). Pre-treatment with 11K2 also blocked repeated NTG-induced behavioral sensitization and the increase in facial pain score (see e.g., FIG. 28B-FIG. 28C and FIG. 29). These data support the therapeutic potential of neutralizing peripheral CCL2 for chronic headache resulting from enhanced NO signaling or repetitive stress.

The CCL2-CCR2 Signaling Interacts with Both CGRP and PACAP Pathways.

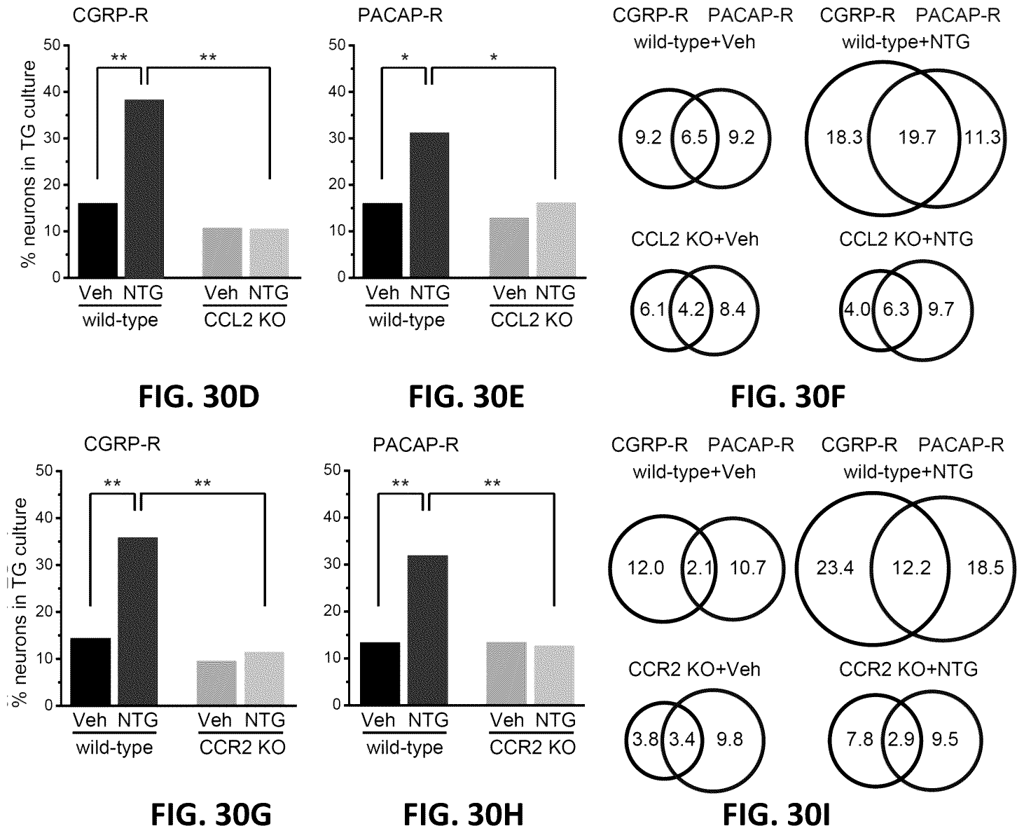

One of the mechanisms through which repeated NTG administration induces chronic headache-related behaviors is to increase the number of TG neurons that respond to neuropeptides CGRP and PACAP (CGRP-R and PACAP-R neurons, respectively). Here, Ccl2 and Ccr2 global KO mice were used to investigate whether endogenous CCL2-CCR2 signaling contributes to the sensitization of TG neurons. Wild-type (C57BL/6J) and KO mice received vehicle or NTG (10 mg/kg, i.p.) every 2 days for 5 times. Two days later, TG neurons were cultured and CGRP- and PACAP-induced intracellular Ca²⁺ increase was measured in individual neurons. TG cultures from vehicle-treated male wild-type and Ccl2 KO mice contained similar numbers of CGRP-R and PACAP-R neurons (see e.g., FIG. 30A-FIG. 30C, Veh). Repeated NTG administration significantly increased the percentages of CGRP-R and PACAP-R neurons in TG cultures from wild-type mice (see e.g., FIG. 30A-FIG. 30C, wild-type+NTG), but did not alter the percentage of CGRP-R or PACAP-R TG neurons in Ccl2 KO mice (see e.g., FIG. 30A-FIG. 30C, CCL2 KO+NTG). Likewise, repeated NTG resulted in higher percentages of CGRP-R and PACAP-R TG neurons in female wild-type mice but not in female Ccl2 KO or Ccr2 KO mice (see e.g., FIG. 30D-FIG. 30I). In conclusion, repeated NTG administration increases the number of TG neurons that express functional CGRP and PACAP receptors via the endogenous CCL2-CCR2 signaling pathway.

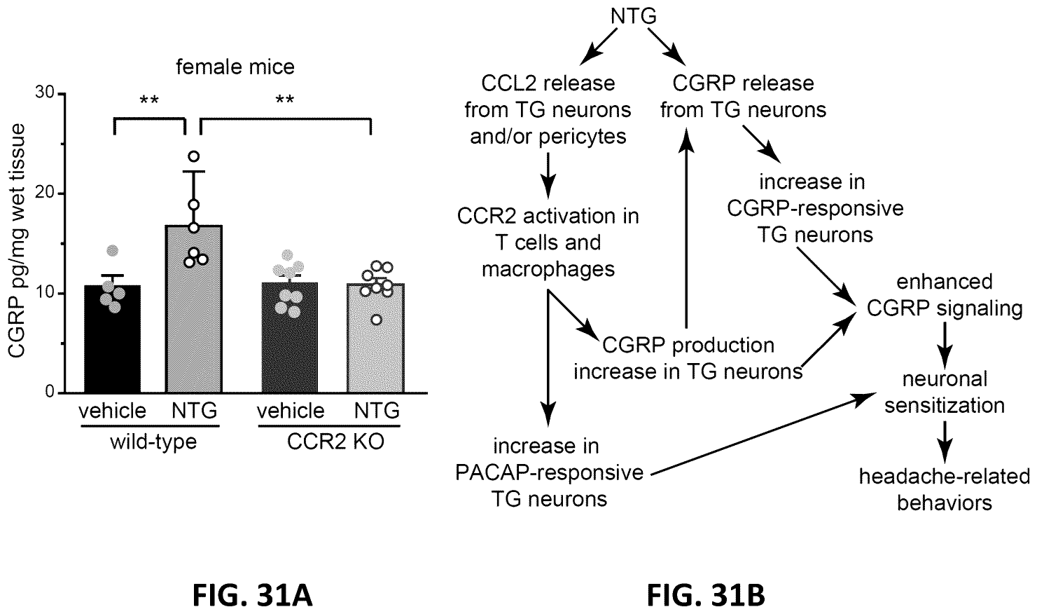

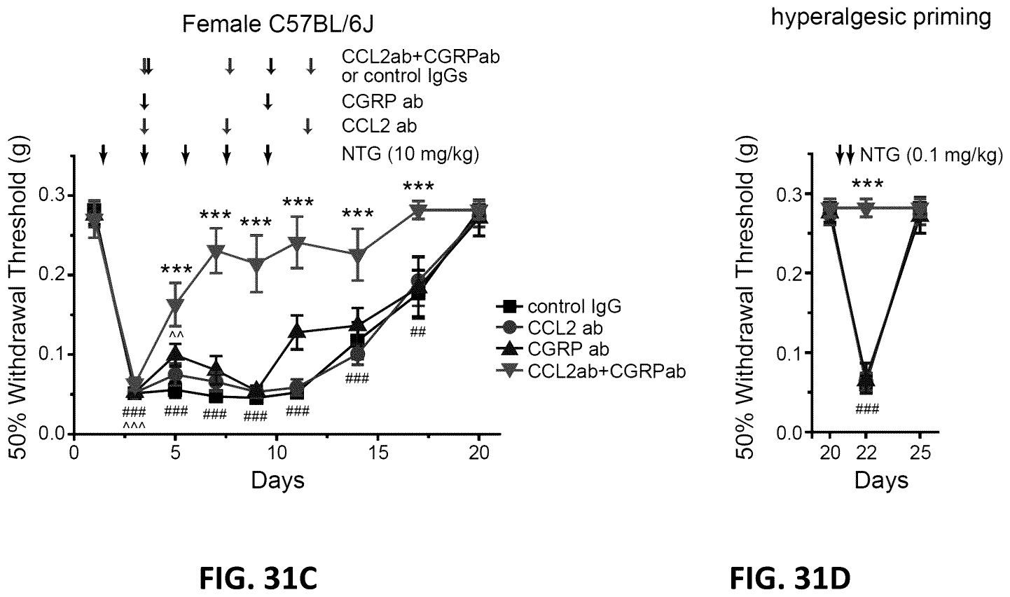

Elevated NO level can stimulated the synthesis and release of CGRP from TG neurons. Repeated NTG administration significantly increased the amount of CGRP in TG tissues from female wild-type (C57BL/6J) mice but not female Ccr2 global KO mice (see e.g., FIG. 31A), indicating that the endogenous CCL2-CCR2 signaling pathway mediates NTG-induced increase in CGRP production. This can further enhance the release of CGRP from TG neurons as well as increase the number of CGRP-R TG neurons, which is regulated by the endogenous CGRP signaling. Together, the data suggest that the endogenous CCL2-CCR2 signaling contributes to chronic headache-related sensitization of TG neurons through enhancing the production of CGRP as well as increasing the level of functional CGRP and PACAP receptors (see e.g., FIG. 31B).

The interaction between CGRP and CCL2 signaling pathways was also investigated at the behavioral level. Mice were treated with a chimeric neutralizing antibody against CGRP (CGRP ab, 30 mg/kg, i.p.) after they exhibited NTG-induced facial skin hypersensitivity (see e.g., FIG. 31C, day 3). This did not alter repeated NTG-induced behaviors (see e.g., FIG. 31C-FIG. 31D, CGRP ab), despite the fact that pre-treating mice with another CGRP neutralizing antibody ALD405 blocked the effects of repeated NTG. A lower dose of the commercially available CCL2 ab (3 mg/kg, i.p.) was also ineffective (see e.g., FIG. 31C-FIG. 31D, CCL2 ab). Conversely, the combination treatment with the CGRP ab and the sub-effective dose of CCL2 ab not only reversed NTG-induced facial mechanical hypersensitivity, but also prevented hyperalgesic priming (see e.g., FIG. 31C-FIG. 31D, CCL2ab+CGRPab). Notably, the combination treatment was more effective than the same duration of high-dose CCL2 ab alone, which only partially attenuated hyperalgesic priming (see e.g., FIG. 6D). These results indicate that inhibition of both peripheral CGRP and CCL2 signaling is more effective in reversing chronic headache-related behavioral sensitization than targeting individual pathways alone.

Discussion

Although the CCL2-CCR2 signaling pathway has been extensively studied in various injury-induced chronic pain models, whether it functionally contributes to chronic headache remains unknown. This knowledge gap was addressed herein. Both NTG-induced acute and persistent behavioral sensitization were abolished in Ccl2 and Ccr2 global KO mice, suggesting that the development of central sensitization underlying NO-induced episodic and chronic headache requires the release of endogenous CCL2 and the activation of CCR2.

Pre-treatment of wild-type mice with CCL2 neutralizing antibodies eliminated chronic headache-related behaviors resulting from repeated NTG administration or repetitive stress, validating a pivotal role of CCL2-CCR2 signaling in both NO— and stress-induced central sensitization. Antibodies do not normally cross the blood brain barrier (BBB), and infusion of NTG in migraine patients triggers delayed headache without compromising BBB integrity. Although repeated NTG administration in mice may increase BBB permeability to small molecules, there is no evidence for enhanced antibody trafficking to CNS. Thus, the results identify the peripheral CCL2-CCR2 signaling pathway as a critical player in chronic headache. Indeed, repeated NTG administration enhanced the strength of CGRP and PACAP signaling in TG neurons from wild-type mice but not neurons from Ccl2 or Ccr2 KO mice, supporting that CCL2-CCR2 signaling contributes to peripheral sensitization, which in turn maintains the central sensitization to elicit chronic headache-related behavioral changes.

Consistent with prior work, Ccl2 mRNA was mainly expressed in TG neurons under control and chronic headache-related conditions. Repeated NTG may trigger Ca²⁺-dependent release of CCL2 stored in large dense-core vesicles from cell bodies in TG and axonal terminals in dura. Additional source of CCL2 may come from mural cells closely associated with dural blood vessels. Many studies report that tissue and nerve injuries increase CCR2 expression in primary afferent neurons, suggesting a paracrine/autocrine CCL2-CCR2 signaling during chronic pain. In contrast, the results indicate that CCR2 is expressed in subsets of macrophages and T cells but not in TG neurons under control and chronic headache-related conditions, supporting the working model that repeated NTG administration releases CCL2 from TG neurons and dural mural cells to activate CCR2 in monocytes/macrophages and T cells in TG and dura.

To functionally identify the cellular targets of CCL2 during chronic headache-related state, Ccr2 was selectively deleted in T cells, myeloid cells, and primary afferent neurons, respectively. Mice with Ccr2 deletion in primary afferent neurons still exhibited NTG-induced behavioral sensitization, exactly the same as control mice, arguing against a contribution of CCL2-CCR2 signaling in TG neurons to headache chronification. Conversely, eliminating Ccr2 in either T cells or myeloid cells abolished repeated NTG-induced facial mechanical hypersensitivity and hyperalgesic priming in both male and female mice. Thus, both CCL2-CCR2 signaling in T cells and macrophages are required to establish chronic headache-related sensitization, either one alone is not sufficient to mediate the effects of repeated NTG administration.

CCL2 is expressed in human TG tissues and pericytes. In human heart, CCR2 is expressed in a distinct subpopulation of macrophages that are maintained through monocyte recruitment and proliferation, and are functionally proinflammatory. CCR2⁺ T cells in human blood belongs to a stable population of effector memory CD4⁺ T cells equipped for rapid activation and cytokine production. Follow-up studies are needed to determine whether CCR2⁺ macrophages and T cells are present in human dura and TG, and if yes, how CCL2-CCR2 signaling modulates their molecular characteristics and functionality.

Many molecular and cellular players have been identified to mediate NTG-induced acute and persistent sensitization. Whether and how they interact with the CCL2-CCR2 signaling in immune cells to initiate and sustain chronic headache-related peripheral and central sensitization requires further investigation. Dura and TG tissues from Ccr2 KO mice contained lower density of macrophages than those from Ccr2 HZ mice, consistent with the notion that CCL2-CCR2 signaling mediates the migration and infiltration of monocytes/macrophages to target tissues. It is possible that NTG-induced CCL2 release from primary afferent neurons and mural cells recruits more CCR2-expressing macrophages to TG and dural perivascular space, as in animal models of paclitaxel-induced peripheral neuropathy and trigeminal nerve injury. Activation of macrophage CCR2 can increase the production of reactive oxygen and carbonyl species, which are known to activate transient receptor potential ankyrin 1 (TRPA1) and transient receptor potential melastatin 8 (TRPM8) channels. This not only activates/sensitizes TG neurons, but also increases the production of reactive oxygen and carbonyl species in TG neurons as the result of TRPA1 activation. These feed forward mechanisms can maintain the peripheral sensitization, eventually leading to central sensitization and chronic headache.

The function of T cells in migraine pathophysiology is not well defined. Herein is provided the first evidence that CCL2-CCR2 signaling in T cells is required to establish the behavioral sensitization related to episodic and chronic headache. Ccr2 KO mice had normal numbers of total T cells and immunosuppressive Treg cells in dura and TG, suggesting that CCL2-CCR2 signaling contributes to chronic headache through regulating the functions of T cells rather than enhancing T cell homing. In a mouse model of T cell-dependent colitis, deletion of Ccr2 does not impair T cell entry into the colons but significantly compromises the production of inflammatory cytokines via the PI3K/Akt signaling cascade. Future in-depth studies are warranted to determine whether CCL2-CCR2 signaling regulates the function of dural and TG T cells through similar or distinct mechanisms, and how this contributes to the activation/sensitization of TG neurons during chronic headache state.

Compared to the remarkable CCL2 and CCR2 upregulation in animals after tissue or nerve injury, the levels of Ccl2 and Ccr2 mRNA were only moderately elevated by repeated NTG administration and did not change at all 1 day after repetitive restraint stress. This raises the possibility that chronic headache and other types of functional pain could arise from baseline CCL2-CCR2 signaling in resident immune cells and may not require a drastic enhancement of CCL2-CCR2 signaling as in injury-induced chronic pain. That CCL2-CCR2 signaling operates at baseline level in individual cells may compel the engagement of both CCR2-expressing macrophages and T cells to obtain sufficient ensemble signaling strength to initiate and sustain neuronal sensitization underlying chronic headache. Alternatively, CCR2 signaling in T cells may indirectly affect the infiltration of monocytes/macrophages to dura and TG through controlling the inflammatory milieu as in the colitis model. In this scenario, the CCL2-CCR2 signaling in T cells and macrophages would work in series rather than in parallel. More work is needed to test these working models.

When mice received CCL2 neutralizing antibodies after repeated NTG- or stress-induced behavioral sensitization was well established, the delayed antibody treatment not only accelerated the resolution of facial skin hypersensitivity, but also suppressed hyperalgesic priming in both models. Moreover, neutralization of CCL2 inhibited NTG- or stress-induced increase in pain aversiveness, suggesting that the peripheral CCL2-CCR2 signaling contributes to both sensory and affective components of headache. Collectively, these results identify peripheral CCL2 and CCR2 as potential therapeutic targets for chronic headache established through two distinct mechanisms. Several blocking antibodies (e.g., carlumab for CCL2 and MLN1202 for CCR2) and small molecule CCR2 antagonists have advanced to phase II clinical trials for other chronic diseases as well as for pain from diabetic neuropathy and knee osteoarthritis (NCT00683423, NCT00689273, NCT05025787), indicating a good safety profile for this class of drugs. That chronic headache may be driven by the CCL2-CCR2 signaling pathway around basal strength implicates that targeting peripheral CCL2 or CCR2 may be more effective for chronic migraine than for injury-induced chronic pain. Notably, the 11K2 antibody that exhibited therapeutic effects in both headache models binds to human CCL2 with higher affinity than with mouse CCL2, which enables a rapid translation from preclinical study to future clinical assessments.

Both CGRP and CCL2-CCR2 signaling are required for the development of NTG-induced acute and persistent sensitization, suggesting a possible interaction between the two pathways. The majority (>90%) of CCL2-expressing TG neurons did not respond to CGRP or PACAP, indicating that the release of CCL2 from TG neurons is not directly regulated by CGRP or PACAP signaling. Whether CGRP and PACAP pathways modulate CCR2 signaling in immune cells under disease state requires further investigation. On the other hand, deletion of Ccl2 or Ccr2 abolished repeated NTG-induced increase in CGRP production and the number of functional CGRP and PACAP receptors, suggesting that CCL2-CCR2 signaling in immune cells enhances the strength of CGRP and PACAP pathways in TG neurons. Future studies need to elucidate the underlying mechanisms.

Although deletion of αCGRP or pre-treatment with the anti-CGRP antibody ALD405 blocked the effects of repeated NTG, neutralizing peripheral CGRP did not reverse NTG-induced persistent sensitization once it is established. This is consistent with the report that CGRP receptor antagonist does not block NO-induced behaviors related to chronic headache, and also in line with the fact that a considerable proportion of chronic migraine patients do not respond to drugs that antagonize the CGRP pathway. Targeting the peripheral CCL2-CCR2 signaling presents a novel treatment option, as it regulates both CGRP and PACAP signaling pathways. Indeed, 16 days of high-dose CCL2 neutralizing antibody treatment fully reversed NTG-induced sensitization. Remarkably, this could be achieved by co-administration of the anti-CGRP antibody with a sub-effective dose of CCL2 antibody for only 8 days, suggesting that inhibition of both peripheral CGRP and CCL2 signaling could benefit more chronic migraine patients than targeting individual pathways alone. The feasibility of testing the combination treatment in future clinical studies is significantly enhanced by the advancement of anti-human CCL2 and CC$2 antibodies to phase II clinical trials for other diseases.

Taken together, the present study supports the model that CCL2 is released from TG neurons and mural cells during migraine episodes. This activates CCR2 in macrophages and T cells and consequently enhances both CGRP and PACAP signaling in TG neurons, ultimately leading to persistent neuronal sensitization and chronic headache. This work not only identifies peripheral CCL2 and CCR2 as potential targets for chronic migraine therapy, but also provides proof-of-concept that inhibition of both peripheral CGRP and CCL2-CCR2 signaling is more effective than blocking either pathway alone.

Herein, the dose of NTG used to sensitize naïve mice is much higher than that used in migraine patients, raising the concern that the behavioral sensitization in mice may result from both headache-related and headache-unrelated mechanisms. However, given that NTG-induced sensitization was abolished in Ccl2/Ccr2 global KO mice, by the systemic anti-CCL2 antibody pre-treatment but not by the periorbital administration of CCR2 antagonist, the results still support the conclusion that peripheral CCL2-CCR2 signaling contributes to chronic headache-related sensitization. In addition to repetitive stress-induced sensitization, the contribution of peripheral CCL2-CCR2 signaling to headache chronification may be further investigated in more chronic migraine models.

Materials and Methods

Mice

All mouse studies followed the ARRIVE guidelines. All procedures were performed in strict accordance with the recommendations in the Guide for the Care and Use of Laboratory Animals of the National Institutes of Health and were approved by the Institutional Animal Care and Use Committee at Washington University in St. Louis. Adult mice (8-14 weeks old, both male and female) were used in the study. In experiments using only wild-type mice, inbred C57BL/6J and outbred CD-1 mice were purchased from Jackson Laboratory and Charles River, respectively. Throughout the study, wild-type mice are referred to as C57BL/6J mice. CD-1 mice were used for experiments involving repetitive restraint stress because the body weight of most 8-14 weeks old C57BL/6J mice could not reach the lower limit for effective restraint (26 gram). Experiments in FIG. 2E-FIG. 2F also used surplus CD-1 mice available in the colony when shipment of C57BL/6J mice was not feasible in 2020 during Covid-19-related research ramp down. The Ccl2 global KO, Ccr2 global KO, CCL2-RFP, CD4Cre, LysMCre and male AdvillinCre (AvCre) breeders were also purchased from the Jackson Laboratory.

To avoid social isolation stress, all mice were group housed (2-5 per cage, same sex) in the animal facility of Washington University in St. Louis on a 12-hour light-dark cycle with constant temperature (23-24° C.), humidity (45%-50%), and food and water ad libitum. All experiments were performed during the light phase (9 am-4 μm).

Ccl2 (Chemokine C—C Motif Ligand 2) and Ccr2 (C—C Motif Chemokine Receptor 2) Global Knockout (KO) Mice

Ccl2 KO and Ccr2 KO breeders were purchased from the Jackson Laboratory (004434 and 027619) and were crossed with wild-type C57BL/6J mice to generate heterozygous (HZ) breeders. The Ccr2 KO mice express enhanced green fluorescent protein (EGFP) from the Ccr2 allele, abolishing endogenous CCR2 expression. Wild-type, HZ and KO mice used in experiments were generated by HZ crossing. Genotypes were determined by PCR of tail DNA.

CCL2-RFP Mice

The CCL2-RFP mice (016849, Jackson Laboratory) contain a modified exon 3 of the mouse Ccl2 gene. A cleavable red fluorescent protein (RFP) mCherry coding region was inserted to the 3′ end of CCL2 coding region, separated by a 19 residue aphthovirus 2A cleavage site. CCL2-RFP mice were generated by crossing homozygous breeders.

Cell-Specific Deletion of Ccr2

The Ccr2^{flox/flox} (Ccr2^{fl/fl}) mouse contains two loxP sites flanking the exon 3 of Ccr2 gene, followed by the enhanced green fluorescent protein (EGFP) cassette. To selectively delete Ccr2 in T cell-, myeloid cell- and primary afferent neurons, Ccr2^{fl/fl} mice were crossed with CD4Cre (022071), LysMCre (004781), and male AdvillinCre (AvCre, 032536, all from Jackson Laboratory) mice to generate CD4CreCcr2^{fl/fl}, LysMCreCcr2^{fl/fl}, and AvCreCcr2^{fl/fl} mice, respectively. Littermates Ccr2^{fl/fl} mice were used as controls. The specificity of Cre-mediated recombination was verified by flow cytometry analysis of peripheral blood cells as well as immunohistochemistry of trigeminal ganglion (TG) and dura tissues.

Mouse Models of Headache-Related Behavioral Sensitization

Repeated nitroglycerin (NTG) administration-induced behavioral sensitization: To model chronic migraine headache, mice received repetitive intraperitoneal (i.p.) injections of nitric oxide (NO) donor NTG (10 mg/kg and 10 ml/kg in saline with 1% propylene glycol) once every 2 days for 5 times as described previously. The control mice received vehicle (saline with 1% propylene glycol, 10 ml/kg) injections every 2 days. Facial mechanical thresholds were measured at baseline and two days after each injection (before the next treatment). After the facial mechanical threshold returned to the baseline level, mice received daily i.p. injections of low-dose NTG (0.1 mg/kg). Facial mechanical thresholds were measured every 2 days to assess hyperalgesic priming.

Repetitive restraint stress-induced behavioral sensitization: Stress is a major trigger of migraine and is a driver of headache chronification. One day after measuring baseline facial mechanical thresholds, adult CD-1 mice (26-35 g) were subjected to 2 hours of restraint stress in the morning for 3 consecutive days as detailed in previous studies. Briefly, mice were guided into individual cylindrical rodent restrainers (Stoelting 51338) facing the acrylic front. The tail was threaded through the moveable disk and the disk was tightened so the mouse was incapable of movement but still maintained normal respiration. Mice were checked every 15-20 minutes (min) to ensure that they did not change the position or suffer from any injury. Sham mice were kept in their home cages without access to water or food during the same period. Responses to facial mechanical stimuli were measured between day 1 and 14 post-stress. After the facial mechanical thresholds returned to the baseline level, mice received one i.p. injection of another NO donor sodium nitroprusside (SNP, 0.3 mg/kg). Facial mechanical thresholds were measured the next day to assess hyperalgesic priming.

Behavioral Tests

Withdrawal thresholds to mechanical stimuli on facial skin: Adult mice were extensively handled by the experimenters for 2 weeks and were well habituated to the test room and the test apparatus before each experiment. The experimenters were blinded to the genotype and the treatments mice received during data collection and analysis.

The hair on the mouse forehead (above and between the eyes) was shaved the day before testing. On the test day, the experimenter gently held the mouse on the palm with minimal restraint and applied the calibrated von Frey filament perpendicularly to the shaved skin, causing the filament to bend for 5 seconds. A positive response was determined by the following criteria as previously described: mouse vigorously stroked its face with the forepaw, head withdrawal from the stimulus, or head shaking. The up-down paradigm was used to determine the 50% withdrawal threshold.

Facial pain score—aversiveness to facial mechanical stimuli: To measure the aversiveness to facial mechanical stimuli, the responses of mice to individual von Frey filaments were scored based on the most aversive behavior they display: 0) no responses; 1) detection: eye squinting, facial skin crinkling and/or a small head shaking; 2) withdrawal: head withdrawal from the filament and/or forepaw stroking of face once; 3) vigorous forepaw stroking of face 2 or more times; and 4) vigorous head shaking and/or attacking the filament.

For C57BL/6J mice, responses were measured to 0.04 gram (g), 0.07 g and 0.16 g von Frey filaments (3 applications each in ascending order). For CD-1 mice, 0.07 g, 0.16 g, and 0.40 g filaments were used. For each experimental group at each time point, responses from all mice to individual von Frey filament were combined to calculate the frequency of individual scores.

For each mouse at each time point, a cumulative pain score was also generated by combining the 9 individual scores.

Drug Administration

On the days that the mouse behaviors were tested, drugs were injected after completing the behavioral tests.

Repeated NTG Administration

NTG (SDM27, Copperhead Chemical, Tamaqua, PA) was freshly diluted from the stock (10% in propylene glycol, aliquoted in airtight glass vials, stored at room temperature and kept in darkness) with saline for every injection.

Mice received intraperitoneal (i.p.) injections of NTG (10 mg/kg) or vehicle (saline with 1% propylene glycol, 10 ml/kg) once every 2 days for 5 times as described previously. Facial mechanical thresholds were measured at baseline and two days after each injection. After the recovery of facial mechanical hypersensitivity, mice received daily i.p. low-dose NTG injections (0.1 mg/kg) to assess hyperalgesic priming.

Repetitive Restraint Stress

CD-1 mice (26-35 g) were subjected to 2 hours of restraint stress for 3 consecutive days. Sham mice were food and water deprived in home cages during the same period. Facial mechanical thresholds were measured before after stress. After the recovery of facial mechanical hypersensitivity, mice received one i.p. injection of another NO donor sodium nitroprusside (SNP, 0.3 mg/kg) to assess hyperalgesic priming.

CCR2 Antagonist Treatment

To block CCR2 signaling, mice received daily i.p. injections of the selective CCR2 antagonist RS504393 (3 mg/kg, Tocris, Ellisville, MO), which was freshly diluted from the stock (4 mg/ml in dimethyl sulfoxide [DMSO] at −20° C.) with saline for every injection. The control group received vehicle injections (saline with 10% DMSO).

To block local CCR2 signaling, mice received 2 daily subcutaneous (s.c.) injections of RS504393 (3 μg in 10 μl vehicle) under periorbital skin. The control mice received vehicle injections (10 μl saline with 3.75% DMSO). When administered intrathecally, this dose of RS504393 significantly attenuated tibial fracture/cast immobilization-induced mechanical allodynia in a mouse model of complex regional pain syndrome.

Neutralizing Antibody Administration

All antibody stocks were stored at 4° C. and diluted in saline before each injection. To neutralize the endogenous CCL2, mice received i.p. injections of the anti-mouse CCL2 antibody (CCL2 ab, 200 μg/mouse, BE185; BioXcell, West Lebanon, NH) every 4 days.

The control group received isotype-matched control immunoglobulin (IgG, 200 μg/mouse, BE0091; BioXcell) in parallel. To test the effect of a lower dose of CCL2 ab, mice received i.p. injections of 3 mg/kg CCL2 ab every 4 days.

Chimeric recombinant mouse IgG1(κ) antibody 11K216 was generated by adapting the VH and VL sequences of humanized 11K2 (see e.g., US 2007/0134236 A1). The 11K2 antibody neutralizes both human and mouse CCL2. Chimeric recombinant mouse IgG1(κ) anti-calcitonin-gene-related peptide (CGRP) antibody (CGRP ab) was generated by adapting the VH and VL sequences of fremanezumab (from IMGT/mAb-DB). The antibodies were produced by Genscript in HD CHO—S cells and purified by MabSelect SuRe LX affinity chromatography with >80% purity and <1.3 EU/mg endotoxin. Mice received i.p. injections of 30 mg/kg 11K2, CGRP ab or isotype matched control IgG (BE0083; BioXcell) every 6 days.

Of note, i.p. injection of 30 mg/kg CGRP ab every 6 days completely blocked mild traumatic brain injury-induced facial skin hypersensitivity in a mouse model of post-traumatic headache.

Primary Culture of Mouse TG Neurons and Ratiometric Ca²⁺ Imaging

Ratiometric Ca²⁺ imaging of dissociated TG neurons was performed as described in a previous study. Briefly, 2 days after the last vehicle or NTG injection, TG tissues were collected and were treated with 2.5 mg/ml collagenase IV for 10 min followed by 2.5 mg/ml trypsin at 37° C. for 15 min. Cells were dissociated by triturating with fire-polished glass pipettes, resuspended in MEM-based culture medium containing 5% fetal bovine serum, 25 ng/ml nerve growth factor (R&D, Minneapolis, MN) and 10 ng/ml glial cell-derived neurotrophic factor (R&D), and seeded on Matrigel-coated coverslips. Ca²⁺ imaging was performed 2 days later. Each experiment contained neurons from at least 3 batches of culture.

Coverslips containing cultured TG neurons were incubated with HBSS/HEPES solution containing 2.5 μM fura-2 AM and 10% Pluronic F-68 (both from Molecular Probes, Eugene, OR) at 37° C. for 45 min to load the ratiometric Ca²⁺ indicator. De-esterification of the dye was carried out by washing the coverslips 3 times with HBSS/HEPES solution and incubating the coverslips in HBSS/HEPES solution in the dark for an additional 15 min at 37° C. Neurons were used for Ca²⁺ imaging experiments within 1 hour after Fura-2 loading. Coverslips with fura-2 loaded neurons were placed in a flow chamber mounted on a Nikon TE2000S inverted epifluorescent microscope and were perfused with room temperature Tyrode's solution (1 ml/min) containing (in mM): 130 NaCl, 2 KCl, 2 CaCl₂), 2 MgCl₂, 25 Hepes, 30 glucose, pH 7.3-7.4 with NaOH, and 310 mosmol/kg H₂O. Differential interference contrast images of neurons were captured to calculate soma diameters from cross-sectional areas off-line. Healthy neurons were chosen based on the differential interference contrast images. Fura-2 was alternately excited by 340 and 380 nm light (Sutter Lambda LS, Sutter Instrument, Navato, CA) and the emission was detected at 510±20 nm by a UV-transmitting 20× objective (N.A. 0.75) and a prime BSI back illuminated sCMOS camera (Photometrics, Tucson AZ). The frame capture period was 50 milliseconds at 1.5 seconds interval. Metamorph software (Molecular Devices, San Jose, CA) was used for controlling and synchronizing the devices as well as image acquisition and analysis. After a 2-3 min baseline measurement in Tyrode's solution, neurons were perfused with 50 nM PACAP1-38 (pituitary adenylate cyclase-activating polypeptide 1-38, Tocris, 1136) for 1 min followed by washing with Tyrode's for 4 min. Subsequently, the coverslip was incubated with 3 μM human αCGRP (Tocris, 3012) for 1 min followed by washing with Tyrode's for 4 min. CGRP and PACAP were freshly diluted from the stock (aliquots at −80° C.) in Tyrode's solution before each experiment.

Regions of interest (ROIs) encompassing individual neurons were defined a priori. The ratio of fluorescence excited by 340 nm divided by fluorescence excited by 380 nm (R_{340/380}) was determined on a pixel-by-pixel basis and was averaged for each ROI. An additional background area was recorded in each field for off-line subtraction of background fluorescence. Peak responses were determined by calculating the relative increase in R_{340/380} above baseline (F₀, the average R_{340/380} during the 2-3 min baseline measurement). A ΔF/F₀>20% was set as the threshold for a response.

To characterize CCL2-expressing TG neurons, TG cultures from CCL2-RFP mice were first perfused with PACAP and CGRP for 1 min as described above. Subsequently, coverslips were perfused with 1 μM capsaicin (21750, Sigma, St. Louis, MO) for 1 min, followed by washing with Tyrode's for 5 min. Neurons were then stained with 3 μg/ml fluorescein Isothiocyanate-conjugated isolectin B4 (1B4, Sigma) for 5 min. The fluorescence on soma membrane was detected after 5 min perfusion with Tyrode's solution to wash off unbound 1B4.

RNA Extraction, Reverse Transcription, and Quantitative PCR (qPCR)

Female CD-1 mice received i.p. vehicle or NTG (10 mg/kg) injections every 2 days for 5 times. Mice were euthanized 2 days after the last injection. Dura and TG tissues were quickly removed, stored in RNAprotect Tissue reagent (Qiagen, Valencia, CA) and then homogenized using a Polytron homogenizer (Qiagen). Dura and TG tissues were also collected from male CD-1 mice 1 day after the last restraint stress session. Total RNA was isolated using a RNeasy mini Kit (Qiagen) and treated with DNase. Reverse transcription was performed using a High-Capacity cDNA Reverse Transcription Kit (Applied Biosystems, Foster City, CA) based on the manufacturer's protocols.

The qPCR reactions were performed with samples in triplicate on an ABI 7500 fast real-time PCR system using the Taqman Gene Expression Master Mix (Applied Biosystems). The primer Taqman probe for mouse Ccl2 (Mm00441242_m1, Applied Biosystems) and mouse Ccr2 (Mm99999051_gH, Applied Biosystems) were used. The real-time qPCR reactions underwent 50 cycles; cycling conditions for these genes were as follows: 2 minutes at 95° C. for denaturing, 3 seconds at 95° C. for annealing, and 30 seconds at 60° C. for extension. Duplicate samples without cDNA (no-template control) for each gene showed no contaminating DNA. Relative Ccl2 and Ccr2 mRNA levels were normalized to glyceraldehyde 3-phosphate dehydrogenase (Gapdh, Mm 99999915_g1, Applied Biosystems) and quantified by use of the comparative CT (ΔΔCT) method for calculating relative quantitation of gene expression.

Determination of CGRP Levels in TG

Female wild-type and Ccr2 global KO mice received i.p. injections of vehicle or NTG (10 mg/kg) once every 2 days for 5 times. One day after the last injection, TG tissues were collected and homogenized (100 mg wet tissue/1 ml) in 50 mM Tris-HCl (pH 7.4) containing 0.2% bovine serum albumin (Sigma) and 1× protease inhibitor cocktail (Sigma). The homogenate was centrifuged at 26,000 g for 30 min at 4° C. The amount of αCGRP in the supernatant was quantified by ELISA according to manufacturer's instructions (Elabscience, E-EL-M0215, Houston, TX). Samples were assayed in duplicate. Each well contained 100 μl of supernatant (approximately 12 μg total protein). The standard curve ranged from 15.63 to 1000 pg/ml of mouse αCGRP. The detection limit was 9.38 pg/ml.

Blood Collection, Antibody Staining and Flow Cytometry

About 100 μl blood was collected from adult Ccr2^{fl/fl}, LysMCreCcr2^{fl/fl} and CD4CreCcr2^{fl/fl} mice by submandibular bleeding. After lysis of red blood cells (420301, Biolegend, San Diego, CA), cells were pelleted and resuspended for antibody staining. To detect EGFP⁺, Ccr2-deficient among T cells, samples were stained with antibodies from Biolegend that recognize mouse CD45 (clone 30-F11, all hematopoietic cells) and CD3ε (clone 145-2C11, total T cells) as well as live/dead dye (2369448, Invitrogen) for 15 min. To detect EGFP⁺, Ccr2-deleted cells among monocytes, cells were stained with antibodies that recognize mouse CD45 and CD11b (clone M1/70, monocytes) as well as live/dead dye. Non-monocytes were stained with PE-conjugated antibodies against mouse CD3ε, B220 (clone RA3-6B2, B cells), Nk1.1 (clonePK136, natural killer cells), ly6G (clone 1A8, neutrophils) and TER119 (clone TER-119, erythroid cells). The frequency of individual cell subpopulations was determined via flow cytometric analysis. Data were collected with LSR Fortessa (Becton Dickinson, Franklin Lakes, NJ) and analyzed with FlowJo (BD FlowJo, Ashland, OR) software.

Tissue Preparation, Immunohistochemistry, RNAscope In Situ Hybridization and Image Analysis

Mice were euthanized with i.p. injection of barbiturate (200 mg/kg) and were transcardially perfused with warm 0.1 M PBS (pH 7.2) followed by cold 4% formaldehyde in 0.1 M phosphate buffer (pH 7.2) for fixation. TG and lumbar L4 dorsal root ganglion (DRG) tissues were collected and sectioned at 15 μm in the transverse plane, collected on Superfrost Plus glass slides in sequence and stored at −20° C.

RNAscope In Situ Hybridization

RNAscope in situ hybridization was performed using the RNAscope fluorescent multiplex assay kit V2 (323100, ACD Bio, Newark, CA) according to manufacturer's user manual. Briefly, sections underwent 4% paraformaldehyde post-fixation, ethanol dehydration, and pretreatments including hydrogen peroxide, target retrieval and RNAscope protease III sequentially. Sections were then hybridized with probes against mouse Ccl2 (311791, ACD Bio) or Ccr2 (501681, ACD Bio) for 2 hours at 40° C. After amplification, signals were detected by horseradish peroxidase based reaction with Opal570 dye (FP1488001KT, Akoya Biosciences, Marlborough, MA). The sections were cover-slipped with ProLong Gold Antifade Mountant (P36934, Invitrogen, Waltham, MA), sealed with nail polish, and stored at 4° C. Images of the entire TG section were captured using a NanoZoomer Whole-Slide Scanner (Hamamatsu, Bridgewater Township, NJ) with the ×20 objective and TRITC laser. Fluorescent intensity was quantified by ImageJ.

Immunohistochemistry

One in every 3 TG or DRG sections were processed for each immunohistochemistry experiment as described previously.9 The sections were dried at room temperature (RT), washed three times in 0.01 M PBS, and incubated in blocking buffer consisting of 0.01 M PBS, 10% normal goat serum (NGS), and 0.3% triton X-100 for 1 hour at RT. Sections were then incubated overnight with primary antibodies diluted in blocking buffer in a humidity chamber at 4° C. After 6 washes (5 min each) in washing buffer containing 0.01 M PBS with 1% NGS and 0.3% triton and 3 rinses in 0.01 M PBS, sections were incubated with blocking buffer for 1 hour, followed by the incubation with fluorescently conjugated secondary antibodies (all from Invitrogen; 1:1000 dilution in blocking buffer) at RT for 1 hour. After washing off the antibodies, sections were rinsed with PBS, cover-slipped using Fluoromount-G Slide Mounting Medium (Electron Microscopy, Hatfield, PA), sealed with nail polish, and stored at 4° C.

The dura was carefully dissected from the skull using forceps after 4 hours fixation, washed 3 times in 0.01 M PBS and stained as whole mount in a 48-well plate. The blocking and washing buffers used for dura staining contained 0.1% triton.

To amplify EGFP and RFP signals in whole-mount dura and TG sections from various transgenic mice, samples were stained with the chicken anti-EGFP antibody (1:1000, AVES Lab, Davis, CA) or the rabbit anti-RFP antibody (1:200, Abcam, Boston, MA). To identify macrophages, samples were stained with a rabbit antibody recognizing ionized calcium binding adaptor molecule 1 (Iba1, 1:1000, Wako; Richmond, VA). To quantify the total number of T cells, samples were stained with the rat anti-mouse CD3 antibody (1:200, clone 17A2, BioLegend). Regulatory T (Treg) cells were identified by the Alexa Fluor 594-conjugated rat anti-mouse CD25 antibody (1:50, clone PC61, BioLegend). B cells and neutrophils were stained with the anti-mouse CD19 (1:100, clone 6D5, BioLegend) and anti-mouse Ly6G (1:200, clone 1A8, BioLegend) antibodies, respectively. Mast cells were stained with Alexa Fluor 488-conjugated avidin (1:400, A21370, Invitrogen).

The dura from CCL2-RFP mice was stained with the rat anti-mouse CD31 antibody (1:1000, BD Pharmingen, San Diego, CA) to identify the endothelial cells lining the blood vessels. Immunofluorescence was observed through a 40× objective on a Nikon TE2000S inverted epifluorescence microscope. Non-overlapping images of TG sections or dura surrounding the middle meningeal artery (MMA) were randomly captured by a prime BSI back illuminated sCMOS camera (Photometrics). The number and the cross-sectional area of Iba1⁺ macrophages were measured using the SimplePCI software (Hamamatsu). To quantify perivascular macrophages, the density of Iba1 staining (the area that exhibit above-threshold fluorescence/ROI) was measured in dura immediately adjacent to MMA.

To quantify T cells in dura, random, non-overlapping images (10 per mouse) were taken in areas surrounding the MMA. To quantify T cells in TG, all cells on individual sections were counted, and the number was multiplied by 3 to obtain the total number of cells per ganglion in each mouse. Images of individual TG sections were captured by the NanoZoomer Whole-Slide Scanner and measured with SimplePCI to verify that the total areas of the sections quantified were comparable between individual mice.

Representative images were adjusted for contrast and brightness using the same parameter within individual experiments. No other manipulations were made to the images. Image analysis was done with experimenters blinded to the experimental groups.

Statistical Analysis

In all experiments, the experimenters were blinded to the genotype and/or the treatments mice received. For behavioral experiments, power analysis was conducted to estimate sample size with >80% power to show an effect size of 0.8, alpha (two-sided) of 0.05, and a simple covariance structure for repeated measures. For Ca²⁺ imaging, all groups were tested in parallel in individual experiments. Each group contained neurons from at least 3 batches of culture. For immunohistochemistry, the sample size was determined based on previous experience.

All data were reported as mean±standard error of the mean. Shapiro-Wilk test was used to test data normality. Origin10 (Origin Lab, Northampton, MA) and Statistica (StatSoft, Tulsa, OK) were used to calculate statistical significance. Comparison between or within experimental groups were assessed by two-tailed t-test, one-way or two-way ANOVA with post hoc Bonferroni test, one-way or two-way repeated-measures ANOVA (RM-ANOVA) with post-hoc Student-Newman-Keuls test, or X² test followed by post hoc Fisher's exact test with Bonferroni correction where appropriate. Differences with p<0.05 were considered statistically significant. The statistical analysis for individual experiments was described in figure legends.

### Example 2: Targeting Peripheral CCL2-CCR2 Signaling to Treat Chronic Migraine and Post-Traumatic Headache

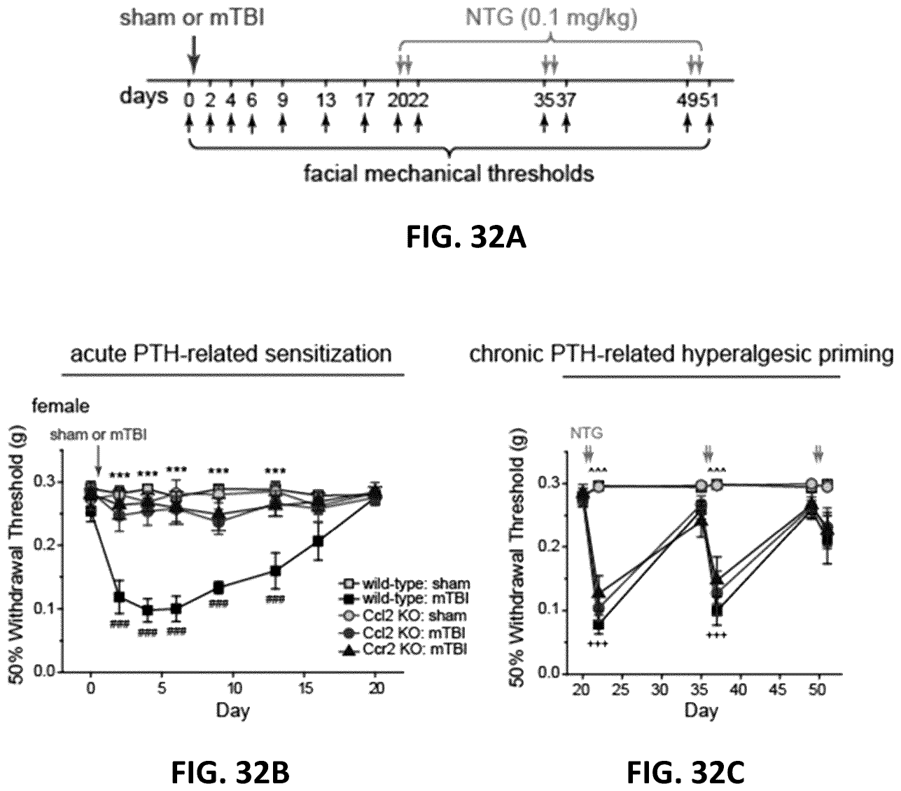

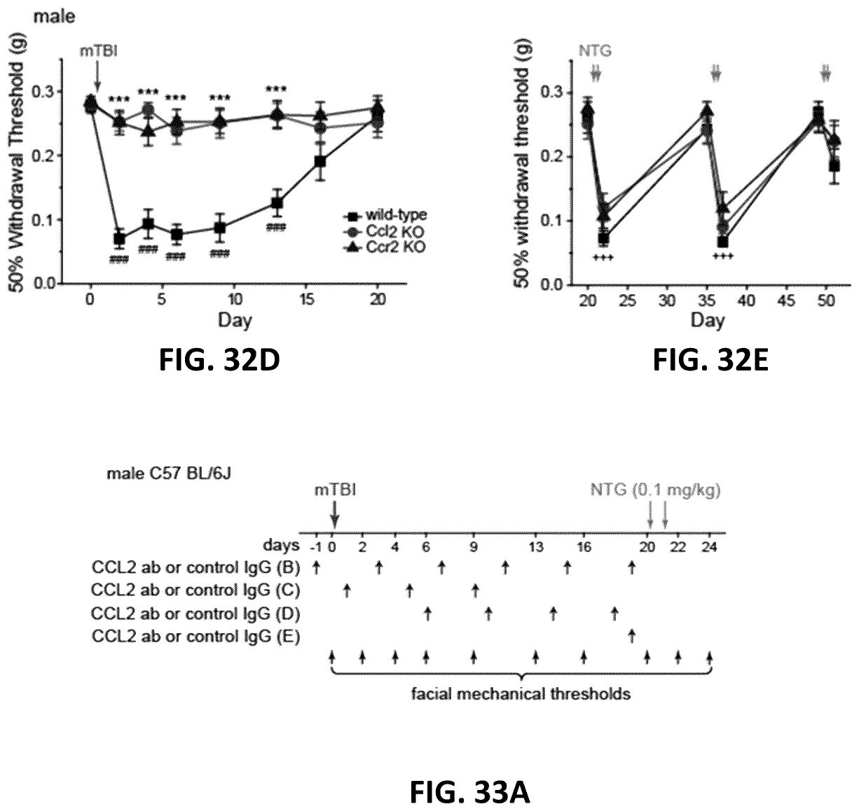

Acute post-traumatic headache (PTH) occurs within the first 7 days after head injury and resolves by 3 months post-mTBI (mild traumatic brain injury); whereas chronic PTH can last for years and often takes on a pattern of daily occurrence in the most severe cases. A previous study reports that the transition from acute to chronic PTH correlates with an increase in serum level of CCL2 between 7 days and 3 months post-mTBI. This prompted investigation of the use the Ccl2 and Ccr2 global KO mice to determine the functional importance of the CCL2-CCR2 signaling pathway in PTH pathophysiology. First, mTBI was induced in female mice by dropping a 21 g weight from a height of 50 cm (see e.g., FIG. 32A-FIG. 32C). In wild-type females, mTBI resulted in a significant reduction of the withdrawal threshold to von Frey filaments on periorbital skin that lasted for 2 weeks (see e.g., FIG. 32B, wild-type: mTBI group), which is mechanistically related to acute PTH. After the facial mechanical thresholds returned to basal level (see e.g., FIG. 32B-FIG. 32C, day 20), 2 injections of nitroglycerin (NTG, a reliable migraine trigger in humans, 0.1 mg/kg i.p., day 20-21) re-established the facial mechanical hypersensitivity (see e.g., FIG. 32C, wild-type: mTBI group, day 22), revealing mTBI-induced hyperalgesic priming that is mechanistically related to chronic PTH. The facial mechanical responses in control mice were not altered by the sham procedure or by NTG (see e.g., FIG. 32B-FIG. 32C, wild-type: sham group). The mTBI-induced hyperalgesic priming lasted for at least 2 weeks after the resolution of acute sensitization as indicated by the repeated administration of NTG every 2 weeks (see e.g., FIG. 32C, wild-type: mTBI group).

Neither Ccl2 nor Ccr2 global KO females exhibited mTBI-induced acute sensitization (see e.g., FIG. 32B, Ccl2 KO:mTBI and Ccr2 KO: mTBI groups). Surprisingly, NTG administration still evoked facial skin hypersensitivity in both Ccl2 and Ccr2 global KO females (see e.g., FIG. 32C, Ccl2 KO:mTBI and Ccr2 KO: mTBI groups). The magnitude and duration of mTBI-induced hyperalgesic priming was comparable between wild-type and KO females (see e.g., FIG. 32C). Similarly, mTBI resulted in both acute sensitization and hyperalgesic priming in wild-type males, whereas male Ccl2 and Ccr2 KO mice only exhibited mTBI-induced hyperalgesic priming (see e.g., FIG. 32D-FIG. 32E).

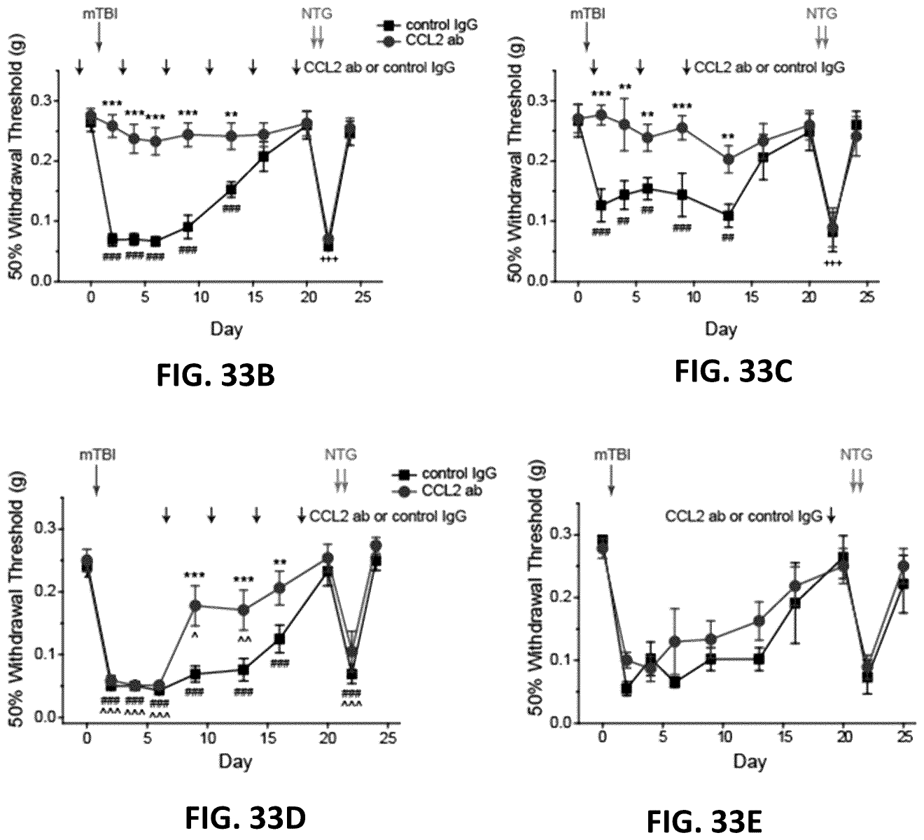

Targeted gene deletion may induce compensatory changes that confound the phenotypic analysis of global KO mice. To address this possibility, the CCL2-CCR2 signaling was inhibited with a neutralizing antibody against mouse CCL2 (CCL2 ab, 200 μg/mouse [approximately 10 mg/kg] every 4 days, see e.g., FIG. 33A). This also allowed for assessment of the relative contributions of central and peripheral CCL2-CCR2 signaling to PTH-related behavioral sensitization. First, male mice received repeated CCL2 ab or control IgG treatment starting one day before mTBI for 3 weeks (see e.g., FIG. 33A-FIG. 33B). Control IgG-treated mice exhibited robust mTBI-induced acute facial skin hypersensitivity and hyperalgesic priming as wild-type mice (see e.g., FIG. 33B, control IgG groups). Starting CCL2 ab treatment before mTBI completely prevented the development of acute sensitization (see e.g., FIG. 33B, CCL2 ab group, day 0-20). However, after the mechanical thresholds returned to baseline level, NTG administration still produced robust facial skin hypersensitivity as in control IgG-treated group (see e.g., FIG. 33B, day 20-24). Starting the CCL2 ab treatment 1 day after mTBI also protected mice from developing acute facial skin hypersensitivity, but mTBI-induced hyperalgesic priming persisted (see e.g., FIG. 33C).

Next, CCL2 ab treatment was started on day 6 post-mTBI, when the acute sensitization was well established (see e.g., FIG. 33D). This not only significantly attenuated mTBI-induced reduction of facial mechanical threshold (see e.g., FIG. 33D, CCL2 ab versus control IgG groups, day 9-13), but also accelerated its recovery to baseline level (see e.g., FIG. 33D, CCL2 ab versus control IgG groups, day 16) without affecting mTBI-induced hyperalgesic priming (see e.g., FIG. 33D, day 20-24). Lastly, mice that underwent mTBI were treated with the CCL2 ab after the complete resolution of acute facial skin hypersensitivity (see e.g., FIG. 33E, day 19) and confirmed that this did not affect the expression of hyperalgesic priming (see e.g., FIG. 33E, day 22). Collectively, these results support that the peripheral CCL2-CCR2 signaling pathway plays a pivotal role in the development and maintenance of acute PTH-related behavioral sensitization, but does not contribute to mTBI-induced hyperalgesic priming that is mechanistically related to chronic PTH.

The results indicate that attenuating peripheral CCL2-CCR2 signaling alone is not effective to alleviate chronic PTH. Likewise, a previous study also reports that a neutralizing antibody against neuropeptide CGRP (calcitonin gene-related peptide) does not inhibit chronic PTH-related behaviors in a rat model. Since previous work showed that inhibition of both peripheral CGRP and CCL2-CCR2 signaling is more effective in reversing chronic migraine-related behaviors than targeting individual pathways alone (see e.g., Example 1), whether this strategy is also effective for chronic PTH-related behaviors was tested.

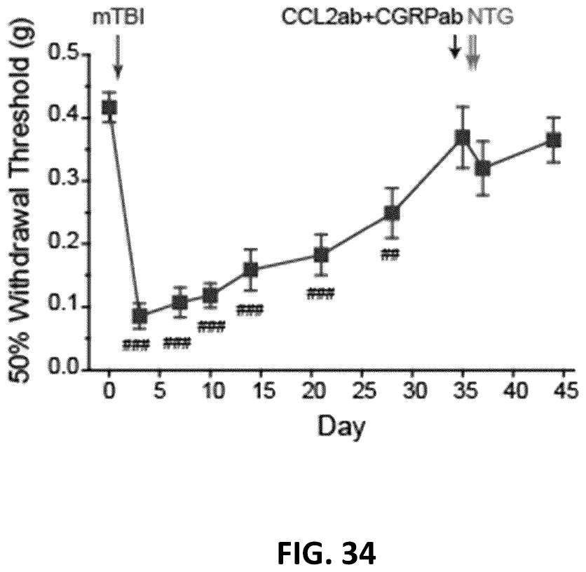

Male CD-1 mice were subjected to mTBI procedure and the acute PTH-related facial skin hypersensitivity was allowed to resolve completely (see e.g., FIG. 34, day 0-35, n=6). Subsequently, mice received the combination treatment with a neutralizing antibody against CGRP (CGRP ab, 30 mg/kg, i.p.) and the CCL2 ab (10 mg/kg, i.p.). Unlike single treatment with the CGRP or CCL2 antibody, NTG administration failed to reveal hyperalgesic priming after the combination treatment (see e.g., FIG. 34, day 35-45), suggesting that inhibition of both peripheral CGRP and CCL2-CCR2 signaling with the combination therapy or with a bi-specific antibody can alleviate chronic PTH.

What is claimed is:

1. A method of treating or preventing a headache disorder in a subject in need thereof comprising administering to the subject a chemokine CC motif ligand 2 (CCL2)-CC chemokine receptor type 2 (CCR2) signaling inhibiting agent.

2. The method of claim 1, wherein the headache disorder is migraine, chronic migraine, chronic headache, stress-induced headache, acute post-traumatic headache (PTH), or chronic PTH.

3. The method of claim 1, wherein the subject has suffered a mild traumatic brain injury (mTBI).

4. The method of claim 1, wherein the CCL2-CCR2 signaling inhibiting agent is an anti-CCL2 or anti-CCR2 antibody.

5. The method of claim 1, wherein the CCL2-CCR2 signaling inhibiting agent is a bispecific antibody against calcitonin gene-related peptide (CGRP) and one of CCL2 or CCR2.

6. The method of claim 1, wherein the CCL2-CCR2 signaling inhibiting agent is a small molecule antagonist of CCR2.

7. The method of claim 6, wherein the small molecule antagonist is RS504393.

8. The method of claim 1, further comprising administering to the subject a CGRP inhibiting agent.

9. The method of claim 8, wherein the CGRP inhibiting agent or the CCL2-CCR2 signaling inhibiting agent is administered to the subject at a sub-effective dose.

10. The method of claim 9, wherein administering the CGRP inhibiting agent or the CCL2-CCR2 signaling inhibiting agent at a sub-effective dose reduces, prevents, or reverses mechanical hypersensitivity or hyperalgesic priming in the subject.

11. The method of claim 8, wherein the CGRP inhibiting agent is an anti-CGRP antibody.

12. The method of claim 1, wherein administering the CCL2-CCR2 signaling inhibiting agent to the subject reduces, prevents, or reverses mechanical hypersensitivity or hyperalgesic priming in the subject.

13. A method of inhibiting chemokine CC motif ligand 2 (CCL2)-CC chemokine receptor type 2 (CCR2) signaling in a subject having a headache disorder comprising administering to the subject a CCL2-CCR2 signaling inhibiting agent.

14. The method of claim 13, wherein the headache disorder is migraine, chronic migraine, chronic headache, stress-induced headache, acute post-traumatic headache (PTH), or chronic PTH.

15. The method of claim 13, wherein the CCL2-CCR2 signaling inhibiting agent is an anti-CCL2 or anti-CCR2 antibody.

16. The method of claim 13, wherein the CCL2-CCR2 signaling inhibiting agent is a bispecific antibody against calcitonin gene-related peptide (CGRP) and CCL2 or CCR2.

17. The method of claim 13, wherein the CCL2-CCR2 signaling inhibiting agent is a small molecule antagonist of CCR2.

18. The method of claim 17, wherein the small molecule antagonist is RS504393.

19. The method of claim 13, further comprising administering to the subject a CGRP inhibiting agent.

20. The method of claim 13, wherein administering the CCL2-CCR2 signaling inhibiting agent to the subject reduces, prevents, or reverses mechanical hypersensitivity or hyperalgesic priming in the subject.
